(19) United States
(12) Patent Application Publication
(10) Pub. No.: US 2016/0205982 A1
(43) Pub. Date: Jul. 21, 2016

(54) LIFE ENHANCING BEVERAGES

(71) Applicant: REOXCYN DISCOVERIES GROUP, INC., North Salt Lake, UT (US)

(72) Inventors: Gary Samuelson, Sandy, UT (US); Daniel Robinson, Salt Lake City, UT (US); David Nieman, North Salt Lake, UT (US); James Pack, Salt Lake City, UT (US); Verdis Norton, Sandy, UT (US)

(21) Appl. No.: 15/023,655

(22) PCT Filed: Sep. 19, 2014
(86) PCT No.: PCT/US2014/056669

§ 371 Date: Mar. 21, 2016

Related U.S. Application Data
(60) Provisional application No. 61/879,863, filed on Sep. 19, 2013.

(51) Int. Cl. A23L 1/025; A23L 2/52; C25B 1/00; A61K 33/14; A23L 2/38; A61K 33/20; C25B 1/04

(52) U.S. Cl. CPC A23L 1/293; A23L 1/237; A23L 2/38; A23L 2/52; A23L 1/025; C25B 1/00; A61K 33/20; A61K 33/14

(57) ABSTRACT

Life enhancing beverages and methods of making and using same are described.

### FIELD

Life enhancing beverages and methods of making the beverages are described.

### SUMMARY

Described generally are life enhancing beverages and methods of making the beverages. Life enhancing beverages can induce, supply, produce, contribute to, supplement, improve, or augment a positive human feature. Positive human features can include drug acceptance, healing, increasing immunity, increasing serum levels of beneficial metabolites such as but not limited to ascorbic acid, and the like.

Life enhancing beverage production systems are described comprising: a mixing apparatus configured to create a solution by mixing water having less than about 0.5 ppm of total dissolved solids and a brine solution having a NaCl concentration of about 537.5 g NaCl/gal; and at least one electrochemical tank including a 7 amp electrode, a recirculation apparatus, and a chilling apparatus configured to chill the solution being electrolyzed.

Methods of forming life enhancing beverages are also described comprising: electrolyzing salinated water having a salt concentration of about 10.75 g NaCl/gal using a set of electrodes with an amperage of about 56 amps to form a life enhancing beverage, wherein the water is chilled below room temperature and the water is circulated during electrolyzing.

In one embodiment, a brine solution is used to salinate the water. The brine solution can have a NaCl concentration of about 537.5 g NaCl/gal.

In one embodiment, the life enhancing beverage can include at least one species selected from 02, H2, Cl2, OCr, HOCl, NaOCl, HCl02, Cl02, HCl03, HCl04, H202, Na+, Cr, H+, H—, OH—, 0 3, 0 4*-, 10, OH*—, HOCl-02*-, HOCl-03, 0 2*-, H02*, NaCl, HCl, NaOH, water clusters, or a combination thereof.

Methods are also described of forming a life enhancing beverage comprising: electrolyzing about 1,000 gal of salinated water having a salt concentration of about 10.75 g NaCl/gal using a set of 8 electrodes with an amperage of about 56 amps to form a life enhancing beverage, wherein the water is chilled to about 4.5° C. to about 5.8° C., the water has less than 0.5 ppm of total dissolved solids before adding brine, and the water is circulated at a rate of about 1,000 gal/hr during electrolyzing.

In other embodiments, methods of increasing athletic performance are described comprising: increasing midocondrial DNA density after administration of a life enhancing beverage including at least one species selected from 0 2, H2, Cl2, OCr, HOCl, NaOCl, HCl02, Cl02, HCl03, HCl04, H202, Na+, er, H+, H—, OH—, 03, 04*-, 10, OH*—, HOCl-02*-, HOCl-03, 02*-, H02*, NaCl, HCl, NaOH, water clusters, or a combination thereof. In still other embodiments, methods of increasing athletic performance are described comprising: reducing a rate of muscle glycogen depletion when exercising after administration of a life enhancing beverage including at least one species selected from 0 2, H2, Cl2, oc1-, HOCl, NaOCl, HCl02, Cl02, HCl03, HCl04, H20 2, Na+, c1-, W, H—, OH—, 0 3, 0 4*-, 10, OH*—, HOCl-02*-, HOCl-03, 0 2*-, H02*, NaCl, HCl, NaOH, water clusters, or a combination thereof. In some embodiments, the administration occurs twice a day or once a day. Each administration can include between about 1 oz and about 16 oz per day. In some embodiments, the rate of muscle glycogen depletion is reduced by about 33% compared to those not treated with the beverage.

In other embodiments, methods of increasing athletic performance are described comprising: increasing time to exhaustion when exercising after administration of a life enhancing beverage including at least one species selected from 0 2, H2, Cl2, OCr, HOCl, NaOCl, HCl02, Cl02, HCl03, HCl04, H202, Na+, Cr, H+, H—, OH—, 03, 04*-, 10, OH*—, HOCl-02*-, HOCl-03, 0 2*-, H02*, NaCl, HCl, NaOH, water clusters, or a combination thereof. In some embodiments, the administration occurs twice a day or once a day. Each administration can include between about 1 oz and about 16 oz per day.

In other embodiments, methods of treating an oxidative stress related disorders are described comprising: administering a life enhancing beverage including at least one species selected from 02, H2, Cl2, OCr, HOCl, NaOCl, HCl02, Cl02, HCl03, HCl04, H202, Na+, Cr, H+, H—, OH—, 0 3, 0 4*-, 10, OH*—, HOCl-02*-, HOCl-03, 0 2*-, H02*, NaCl, HCl, NaOH, water clusters, or a combination thereof to a patient experiencing oxidative stress; and treating the oxidative stress related disorder. In some embodiments, the administration occurs twice a day or once a day. Each administration can include between about 1 oz and about 16 oz per day. In other embodiments, the oxidative stress related disorder is diabetes, cardiovascular disease, or obesity.

In other embodiments, methods of treating a reduced mitochondrial DNA disorder are described comprising: administering a life enhancing beverage including at least one species selected from 02, H2, Cl2, OCr, HOCl, NaOCl, HCl02, Cl02, HCl03, HCl04, H202, Na+, Cr, H+, H—, OH—, 0 3, 0 4*-, 10, OH*—, HOCl-02*-, HOCl-03, 0 2*-, H02*, NaCl, HCl, NaOH, water clusters, or a combination thereof to a patient experiencing the reduced mitochondrial DNA disorder; increasing mitochondrial DNA density; and treating the reduced mitochondrial DNA disorder. In some embodiments, the administration occurs twice a day or once a day. Each administration can include between about 1 oz and about 16 oz per day. In other embodiments, the reduced mitochondrial DNA disorder is sacropenia, diabetes, Alzheimer's disease, Parkinson's disease, neurological disease, muscle loss due to aging, obesity, or cardiovascular disorders.

Also described are methods of scaling up a process for forming a life enhancing beverage comprising: forming a standard life enhancing beverage reference standard in a 1 L container using 0.9% isotonic saline solution and applying 3 amps thereto; using the standard life enhancing beverage as a reference standard for the production of a life enhancing beverage containing at least one reaction product, wherein the reaction product includes 02, H2, Cl2, ocr, HOCl, NaOCl, HCl02, Cl02, HCl03, HCl04, H20 2, Na+, Cr, H+, H—, OH—, 0 3, 0 4*-, 10, OH*—, HOCl-02*-, HOCl-03, 0 2*-, H02*, NaCl, HCl, NaOH, water clusters; and preparing a second life enhancing beverage which has an equivalent amount the of at least one reaction product as the standard life enhancing beverage such that the standard life enhancing beverage is used as a reference standard and the amounts of the of at least one reaction product in the standard life enhancing beverage are a target amount of the of at least one reaction product for the second life enhancing beverage wherein the second life enhancing beverage is made using brine solution having a NaCl concentration of about 537.5 g NaCl/gal in tanks which hold 180 gallons. In one embodiment, a pulsed current can be applied when forming the life enhancing beverage and the second life enhancing beverage.

### BRIEF DESCRIPTION OF THE DRAWINGS

FIG. 1 is a flow chart of a process as described herein.

FIG. 2 illustrates an example diagram of the generation of various molecules at the electrodes, the molecules written between the electrodes depict the initial reactants and those on the outside of the electrodes depict the molecules/ions produced at the electrodes and their electrode potentials.

FIG. 3 illustrates a plan view of a process and system for producing a life enhancing beverage according to the present description.

FIG. 4 illustrates an example system for preparing water for further processing into a life enhancing beverage.

FIG. 5 illustrates a Cl35 spectrum of NaCl, NaClO solution at a pH of 12.48, and the beverage.

FIG. 6 illustrates a ¹H NMR spectrum of a beverage as described.

FIG. 7 illustrates a 31P NMR spectrum of DIPPMPO combined with the beverage.

FIG. 8 illustrates a mass spectrum showing a parent peak and fragmentation pattern for DIPPMPO with m/z peaks at 264, 222, and 180.

FIG. 9 illustrates oxygen/nitrogen ratios for a beverage described herein compared to water and NaClO.

FIG. 10 illustrates chlorine/nitrogen ratios for a beverage described herein compared to water and NaClO.

FIG. 11 illustrates ozone/nitrogen ratios for a beverage described herein compared to water and NaClO.

FIG. 12 illustrates the carbon dioxide to nitrogen ratio of a beverage as described herein compared to water and NaClO.

FIG. 13 illustrates an EPR splitting pattern for a free electron.

FIG. 14 illustrates a flow chart of a mouse study as described in Example 3.

FIG. 15 is a flow chart showing a total overview of the mouse preparation and study.

FIG. 16A illustrates mice grouped into placebo and ASEA treatment versus run time. FIG. 16B illustrates rate of muscle glycogen depletion in mice grouped into placebo and ASEA treatment versus run time.

FIG. 17A illustrates the fold change relate to ASEA of different mouse groups. FIG. 17B illustrates the fold change difference between ASEA sedentary (non-running) and ASEA running groups.

FIG. 18 illustrates different mouse groups versus the amount of liver SOD produced.

FIGS. 19A and 19B illustrate different mouse groups versus oxidized glutothione.

FIG. 20 illustrates different mouse groups versus fold change for IL-6 and TNF-alpha.

FIG. 21 illustrates global metabolic scores between treatment conditions of Example 4.

FIGS. 22A-D illustrate ASEA and placebo groups versus least means square area for different metabolic products.

FIG. 23 illustrates intermediates and products of the Krebs cycle with and without ASEA pre-, post-, and 1 hr post-exercise.

FIG. 24 illustrates affects of ASEA on ascorbic acid both acutely and chronically.

FIG. 25 illustrates a flow chart of the protocol for the study outlined in Example 5.

FIG. 26 illustrates results based on the 9 samples collected from each individual in the study of Example 5 comparing A-B ratios between conditions.

FIG. 27 illustrates a comparison of A-B ratios between conditions 30 minutes post ingestion.

FIG. 28 illustrates a comparison of A-B ratios between conditions 1.5 hours post ingestion.

FIG. 29 illustrates a comparison of A-B ratios between conditions 3.5 hours post ingestion.

FIG. 30 illustrates a comparison of A-B ratios between conditions 24 hours post ingestion.

FIG. 31 illustrates a flow chart of the human running performance study protocol.

FIG. 32 illustrates a flow chart of a 12-week, randomized trial performed accord to the protocol of Example 7.

FIG. 33 illustrates a graph of VC0₂ versus V0₂ resulting from the study in Example 8.

FIG. 34 illustrates cell images for each culture results of HMVEC-L Cells p65 subunit NF-kB screen for toxicity.

FIG. 35 illustrates results for P-Jun screen for toxicity.

FIG. 36 illustrates a graph showing the reduction of oxidants over time an 11 minute interval (RFU units on vertical scale).

FIG. 37 illustrates a graph showing antioxidant activity over time an 11 minute interval.

FIG. 38 illustrates nuclear staining patterns for results of HMVEC-L Nuclear Accumulation of NRF2.

FIG. 39 illustrates serum-starved cell cultures exposed to low-concentration ASEA.

FIG. 40 illustrates a western blot validation of NRF2 nuclear accumulation following ASEA treatment.

FIG. 41 illustrates results for proliferation of murine and HMVEC-L cells and LOH activity following ASEA treatment.

FIG. 42 illustrates further results for proliferation of murine and HMVEC-L cells and LOH activity following ASEA treatment.

FIG. 43 illustrates results of HMVEC-L viability exposed high-concentration ASEA and to escalating amounts of Cachexin stressor.

FIG. 44 illustrates results of concentration-dependent response of HMVEC-L cells to Cachexin insult.

### DETAILED DESCRIPTION

Described herein are life enhancing beverages. The life enhancing beverages generally include at least one reactive oxygen species. In other embodiments, the beverages can include chlorine, OCl⁻ and/or O⁻². In some embodiments, the life enhancing beverages can have a saline concentration of about 0.15% w/v.

Methods of forming these beverages are also described. The methods generally include electrolyzing a saline solution at set conditions to produce a life enhancing beverage.

The life enhancing beverages can induce, supply, produce, contribute to, supplement, improve, or augment a positive human feature. Positive human features can include drug acceptance, healing, increasing immunity, increasing serum levels of beneficial metabolites such as but not limited to ascorbic acid, and the like.

ROS superoxide free radicals (OO*— and OOH*) and hydroxyl free radicals (OH*) can have a short half-life in aqueous solutions (t(½)<2 ms). In one embodiment described herein is a method to produce large-scale concentrations of these biologically active ROS components in aqueous solutions with half lives of, for example, several years, sufficient for long-term storage. Such stable complexes and compositions also include reductive components, such that the combined composition is of neutral pH. The produced compositions may not result in any toxicity in vitro and in vivo.

A method of production can include one or more of the steps of (1) preparation of an ultra-pure homogeneous solution of sodium chloride in water, (2) temperature control and flow regulation through a set of inert catalytic electrodes and (3) a modulated electrolytic process that results in the formation of such stable molecular moieties and complexes. In one embodiment, such a process includes all these steps.

The electro-catalytic process that forms such moieties can rely heavily on the purity and molecular homogeneity of the reactants as they make contact with the local reactive surfaces of the electrodes. Preparation of the saline solution can be a critical step in the process. The saline generally should be free from contaminants, both organic and inorganic, and homogeneous down to the molecular level. In particular, metal ions can interfere with the electro-catalytic surface reactions, and thus contamination of the water or saline by metals should be avoided.

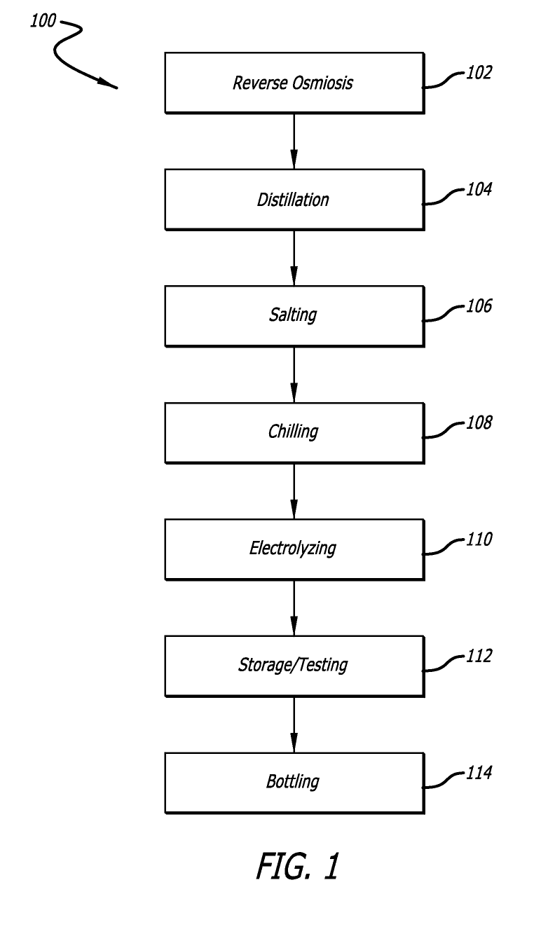

With this in mind, the first step in such a process 100 is an optional reverse osmosis procedure 102 (FIG. 1). Water can be supplied from a variety of sources, including but not limited to municipal water, filtered water, nanopure water, or the like. Municipal water, for example, can be highly variable depending on the municipal water source (e.g. stream or river versus surface or underground reservoir water), the method of sterilizing the water prior to distribution (e.g., UV light), chemicals used to treat the water, and the like. Regardless of the source of water, optionally, reverse osmosis can be used to reproducibly clean the water.

The reverse osmosis process can vary, but can provide water having a total dissolved solids content of less than about 10 ppm, about 9 ppm, about 8 ppm, about 7 ppm, about 6 ppm, about 5 ppm, about 4 ppm, about 3 ppm, about 2 ppm, about 1 ppm, 0.5 ppm, less than about 10 ppm, less than about 9 ppm, less than about 8 ppm, less than about 7 ppm, less than about 6 ppm, less than about 5 ppm, less than about 4 ppm, less than about 3 ppm, less than about 2 ppm, or less than about 1 ppm.

The temperature of the reverse osmosis process can be preformed at a temperature of about 5° C., about 10° C., about 15° C., about 20° C., about 25° C., about 30° C., or about 35° C., from about 5° C. to about 35° C., from about 10° C. to about 25° C., from about 5° C. to about 25° C., from about 10° C. to about 35° C., from about 20° C. to about 30° C., less than about 35° C., less than about 30° C., less than about 25° C., less than about 20° C., greater than about 5° C., greater than about 10° C., greater than about 15° C., or greater than about 20° C.

The process can further output cleansed water at a speed of about 1 gal/min, about 1.5 gal/min, about 2 gal/min, about 2.5 gal/min, about 3 gal/min, about 3.5 gal/min, about 4 gal/min, about 4.5 gal/min, about 5 gal/min, about 5.5 gal/min, about 6 gal/min, about 6.5 gal/min, about 7 gal/min, about 7.5 gal/min, about 8 gal/min, about 8.5 gal/min, about 9 gal/min, about 9.5 gal/min, about 10 gal/min, about 11 gal/min, or about 12 gal/min, between about 1 gal/min and about 12 gal/min, between about 2 gal/min and about 10 gal/min, between about 4 gal/min and about 8 gal/min, between about 1 gal/min and about 8 gal/min, between about 4 gal/min and about 12 gal/min, at least about 1 gal/min, at least about 2 gal/min, at least about 4 gal/min, or any range bound by any of these values.

The reverse osmosis step can be repeated as needed to achieve a particular total dissolved solids level.

Whether the optional reverse osmosis step is utilized, an optional distillation step 104 can be performed.

The distillation process can vary, but can provide water having a total dissolved solids content of less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, less than about 1 ppm, less than about 0.9 ppm, less than about 0.8 ppm, less than about 0.7 ppm, less than about 0.6 ppm, less than about 0.5 ppm, less than about 0.4 ppm, less than about 0.3 ppm, less than about 0.2 ppm, or less than about 0.1 ppm.

The temperature of the distillation process can be preformed at a temperature of about 5° C., about 10° C., about 15° C., about 20° C., about 25° C., about 30° C., or about 35° C., from about 5° C. to about 35° C., from about 10° C. to about 25° C., from about 5° C. to about 25° C., from about 10° C. to about 35° C., from about 20° C. to about 30° C., less than about 35° C., less than about 30° C., less than about 25° C., less than about 20° C., greater than about 5° C., greater than about 10° C., greater than about 15° C., or greater than about 20° C. In one embodiment, the distillation can be run at about room temperature.

The distillation process can further output distilled water at a speed of about 250 gal/hr, about 280 gal/hr, about 300 gal/hr, about 310 gal/hr, about 320 gal/hr, about 330 gal/hr, about 335 gal/hr, about 340 gal/hr, about 345 gal/hr, about 350 gal/hr, about 355 gal/hr, about 360 gal/hr, about 365 gal/hr, about 370 gal/hr, about 375 gal/hr, about 380 gal/hr, about 385 gal/hr, about 390 gal/hr, about 395 gal/hr, about 400 gal/hr, or about 420 gal/hr, between about 340 gal/min and about 420 gal/min, between about 250 gal/min and about 365 gal/min, between about 300 gal/min and about 400 gal/min, between about 250 gal/hr and about 420 gal/hr, between about 335 gal/hr and about 385 gal/hr, at least about 250 gal/hr, at least about 280 gal/hr, at least about 300 gal/hr, or any range bound by any of these values.

The distillation step can be repeated as needed to achieve a particular a total dissolved solids level. After water has been subjected to reverse osmosis, distillation, both or neither, the level of total dissolved solids in the water can be less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, less than about 1 ppm, less than about 0.9 ppm, less than about 0.8 ppm, less than about 0.7 ppm, less than about 0.6 ppm, less than about 0.5 ppm, less than about 0.4 ppm, less than about 0.3 ppm, less than about 0.2 ppm, or less than about 0.1 ppm. The amount of total dissolved solids in the water can be an important aspect in the final product as some solids can create unwanted side products during electrolyzing. Also, unwanted solids can also prevent full or efficient electrolyzing. As such, a reduction of total dissolved solids in the water, in some embodiments, is less than about 0.5 ppm.

The reverse osmosis, distillation, both or neither can be preceded by a carbon filtration system which can remove oils, alcohols, and other volatile chemical residuals and particulates that can be present in municipal water or otherwise. Also, before reverse, osmosis, distillation, both or neither, water can be passed through resin tanks to remove dissolved minerals.

Purified water can be used directly with the systems and methods described herein. For example, if purified water is used that has a total dissolved solids concentration of less than about 0.5 ppm, neither reverse osmosis nor distillation needs to be used. In other embodiments, if semi-purified water is used, only one of the processes may be used.

In one embodiment, contaminants can be removed from a commercial source of water by the following procedure: water flows through an activated carbon filter to remove the aromatic and volatile contaminants and then undergoes Reverse Osmosis (RO) filtration to remove dissolved solids and most organic and inorganic contaminants. The resulting filtered RO water can contain less than about 8 ppm of dissolved solids. Most of the remaining contaminants can be removed through a distillation process, resulting in dissolved solid measurements less than 1 ppm. In addition to removing contaminants, distillation may also serve to condition the water with the correct structure and Oxidation Reduction Potential (ORP) to facilitate the oxidative and reductive reaction potentials on the platinum electrodes in the subsequent electro-catalytic process.

After water has been subjected to reverse osmosis, distillation, both or neither, a salt is added to the water in a salting step 106. The salt can be unrefined, refined, caked, de-caked, or the like. In one embodiment, the salt is sodium chloride (NaCl). In some embodiments, the salt can include an additive. Salt additives can include, but are not limited to potassium iodide, sodium iodidie, sodium iodate, dextrose, sodium fluoride, sodium ferrocyanide, tricalcium phosphate, calcium carbonate, magnesium carbonate, fatty acids, magnesium oxide, silicone dioxide, calcium silicate, sodium aluminosilicate, calcium aluminosilicate, ferrous fumarate, iron, or folic acid. Any of these additives can be added at this point or at any point during the described process. For example, the above additives can be added just prior to bottling.

Salt can be added to water in the form of a brine solution. Brine can be formed at a salt ratio of about 500 g NaCl/gal water, about 505 g NaCl/gal water, about 510 g NaCl/gal water, about 515 g NaCl/gal water, about 520 g NaCl/gal water, about 525 g NaCl/gal water, about 530 g NaCl/gal water, about 535 g NaCl/gal water, about 536 g NaCl/gal water, about 537 g NaCl/gal water, about 538 g NaCl/gal water, about 539 g NaCl/gal water, about 540 g NaCl/gal water, about 545 g NaCl/gal water, about 550 g NaCl/gal water, about 555 g NaCl/gal water, about 560 g NaCl/gal water, about 565 g NaCl/gal water, about 570 g NaCl/gal water, about 575 g NaCl/gal water, about 580 g NaCl/gal water, between about 500 g NaCl/gal water and about 580 g NaCl/gal water, between about 520 g NaCl/gal water and about 560 g NaCl/gal water, or between about 535 g NaCl/gal water and about 540 g NaCl/gal water. In one embodiment, the ratio can be about 537.5 g NaCl/gal water.

Brine can be formed by adding NaCl to water in a tank. For example, for a 500 gal tank, about 475 gal of water can be added to the tank and a proper amount of NaCl is added to achieve a desired ratio. The brine solution can then be thoroughly mixed for about 30 min, about 1 hr, about 6 hr, about 12 hr, about 1 day, about 2 days, about 3 days, about 4 days, about 5 days, about 6 days, about 7 days, about 8 days, about 9 days, about 10 days, or longer.

To mix the brine solution, a physical mixing apparatus can be used or a circulation or recirculation can be used. A tank can circulate or recirculate solution at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. The amount of mixing time or type of mixing used can vary. However, in some embodiments, at the end of mixing, all the salt can be dissociated.

In one embodiment, pure pharmaceutical grade sodium chloride is dissolved in the prepared distilled water to form a 15 wt % sub-saturated brine solution and continuously re-circulated and filtered until the salt has completely dissolved and all particles>0.1 microns are removed. This step can take several days. The filtered, dissolved brine solution is then injected into tanks of distilled water in about a 1:352 ratio (salt:water) in order to form a 0.3% saline solution. In one embodiment, a ratio 10.75 g of salt per 1 gallon of water can be used to form the beverage. In another embodiment, 10.75 g of salt per 3,787.5 g of water can be used to form the beverage. This solution then can be allowed to re-circulate and diffuse until homogeneity at the molecular scale has been achieved. The diffusion coefficient of this brine in distilled water is about 1.5×10−9 m²/s at 25° C. The Einstein diffusion time (t=<x>2/2D) can then be used to determine the time it will take for the sodium chloride ions to diffuse completely in the saline solution. About 5 minutes may be required for molecules to completely diffuse 1 mm from concentration centers, and 500 minutes are required for the molecules to diffuse 1 cm based on the above approximation.

Mechanical mixing through recirculation can be required to speed diffusion. With full-tank circulation every hour for 24 hours, sodium chloride concentration centers can be homogeneous down to about the 1 cm level another 24 hours may be required to achieve diffusive homogeneity down to the molecular scale throughout the entire saline solution. The entire homogenization process can take an average of about 36 hours. Mixing discs, with microporous material, can be put in the recirculation lines to accelerate the mixing process, higher temperatures can also accelerate this process. In one embodiment, all materials and pumps that might come into contact with the saline solution can be of pristine high density hydrophobic polymer material or glass to prevent contamination. Also, tanks can remain closed to prevent atmospheric contamination.

In one embodiment, the homogenous saline solution is chilled to about 4.8±0.5° C. This temperature may be critical because higher temperatures can increase ROS content and lower temperatures can increase hypochlorite (RS) and possibly free chlorine content during processing. Correct balance can require precisely controlled temperature at the electro-catalytic surfaces. Careful temperature regulation during the entire electro-catalytic process is required as thermal energy generated from the electrolysis process itself may cause heating. In one embodiment, process temperatures at the electrodes can be constantly cooled and maintained at about 4.8° C. throughout electrolysis.

Brine can then be added to the previously treated water or to fresh untreated water to achieve a NaCl concentration of about 1 g NaCl/gal water, about 2 g NaCl/gal water, about 3 g NaCl/gal water, about 4 g NaCl/gal water, about 5 g NaCl/gal water, about 6 g NaCl/gal water, about 7 g NaCl/gal water, about 8 g NaCl/gal water, about 9 g NaCl/gal water, about 10 g NaCl/gal water, about 10.25 g NaCl/gal water, about 10.50 g NaCl/gal water, about 10.75 g NaCl/gal water, about 11 g NaCl/gal water, about 12 g NaCl/gal water, about 13 g NaCl/gal water, about 14 g NaCl/gal water, about 15 g NaCl/gal water, about 16 g NaCl/gal water, about 17 g NaCl/gal water, about 18 g NaCl/gal water, about 19 g NaCl/gal water, about 20 g NaCl/gal water, about 21 g NaCl/gal water, about 22 g NaCl/gal water, about 23 g NaCl/gal water, about 24 g NaCl/gal water, about 25 g NaCl/gal water, between about 1 g NaCl/gal water and about 25 g NaCl/gal water, between about 8 g NaCl/gal water and about 12 g NaCl/gal water, or between about 4 g NaCl/gal water and about 16 g NaCl/gal water.

Once brine is added to water at an appropriate amount, the solution can be thoroughly mixed for about 30 min, about 1 hr, about 6 hr, about 12 hr, about 24 hr, about 36 hr, about 48 hr, about 60 hr, about 72 hr, about 84 hr, about 96 hr, about 108 hr, about 120 hr, about 132 hr, or longer, no less than about 12 hr, no less than about 24 hr, no less than about 36 hr, no less than about 48 hr, no less than about 60 hr, no less than about 72 hr, no less than about 84 hr, no less than about 96 hr, no less than about 108 hr, no less than about 120 hr, or no less than about 132 hr.

The temperature of the liquid during mixing can be at room temperature or controlled at a temperature of about 20° C., about 25° C. about 30° C. about 35° C. about 40° C. about 45° C. about 50° C. about 55° C., about 60° C., between about 20° C. and about 40° C., between about 30° C. and about 40° C., between about 20° C. and about 30° C., between about 25° C. and about 30° C., or between about 30° C. and about 35° C.

To mix the solution, a physical mixing apparatus can be used or a circulation or recirculation can be used. A tank can circulate or recirculate solution at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. The amount of mixing time or type of mixing used can vary. In some embodiments, the mixing time is sufficient to allow complete dissociation of the NaCl.

The salt solution can then be chilled in a chilling step 108. The temperature of the chilled solution can be about 0° C., about 1° C. about 2° C. about 3° C. about 4° C. about 5° C. about 6° C. about 7° C., about 8° C., about 9° C., about 10° C., about 11° C., about 12° C., between about 0° C. and about 10° C., between about 0° C. and about 5° C., between about 5° C. and about 10° C., between about 0° C. and about 7° C., or between about 2° C. and about 5° C. In one embodiment, the chilled solution can have a temperature of between about 4.5° C. and about 5.8° C.

For large amounts of solution, various chilling and cooling methods can be employed. For example cryogenic cooling using liquid nitrogen cooling lines can be used. Likewise, the solution can be run through propylene glycol heat exchangers to achieve the desired temperature. The chilling process can take about 30 min, about 1 hr, about 2 hr, about 3 hr, about 4 hr, about 5 hr, about 6 hr, about 7 hr, about 8 hr, about 9 hr, about 10 hr, about 12 hr, about 14 hr, about 16 hr, about 18 hr, about 20 hr, about 22 hr, about 24 hr, between about 30 min and about 24 hr, between about 1 hr and about 12 hr, at least about 30 min, at least about 6 hr, at most about 24 hr, or any range created using any of these values to get the solution from room temperature to a desired chilled temperature. The chilling time can vary depending on the amount of liquid, the starting temperature and the desired chilled temperature. A skilled artisan can calculate the time required to chill a solution as described.

Products from the anodic reactions also need to be effectively transported to the cathode to provide the reactants necessary to form the stable complexes on the cathode surfaces. This requires that there be an active flow of liquid from the anode to the cathode during electrolysis. Maintaining a high degree of homogeneity in the fluids circulated between the catalytic surfaces can also be of high importance. A constant steady uniform flow of about 2-8 ml/cm²*sec can be optimal between the anode and the cathode, with typical mesh electrode distances 2 cm apart in large tanks. This flow is maintained, in part, by the convective flow of gasses released from the electrodes during electrolysis. In the small liter units, this convective flow alone can be sufficient to maintain proper circulation, in larger units, powered flow-control is necessary. Insufficient mixing or non-uniform circulation in the tanks can also cause inhomogeneities in the stream of reactants supplied to the electrodes, this will result in unpredictable and inconsistent results for the electrode reactions themselves.

For example, a build-up of excessive ROS at the electrodes can cause over-processing and build-up of undesirable reaction products in the neighborhood of the electrodes that cannot be reversed by mixing with under-processed solution elsewhere in the tank. This is also true when mixing over-processed products with under-processed products made from different tanks. For consistent product, a constant homogeneous flow of reactants should pass through the electrodes, and a consistent solution should be maintained throughout the volume of the tank. Proper flow and mixing are required. This is one of the major obstacles to scale-up the process.

The mixed solution chilled or not can then undergo electrochemical processing through the use of at least one electrode in an electrolyzing step 110. Each electrode can be or include a conductive metal. Metals can include, but are not limited to copper, aluminum, titanium, rhodium, platinum, silver, gold, iron, a combination thereof or an alloy such as steel or brass. The electrode can be coated or plated with a different metal such as, but not limited to aluminum, gold, platinum or silver. In one embodiment, each electrode is formed of titanium and plated with platinum.

In one embodiment, rough platinum-plated mesh electrodes in a vertical, coaxial, cylindrical geometry can be optimal, with not more than 2.5 cm, not more than 5 cm, not more than 10 cm, not more than 20 cm, or not more than 50 cm separation between the anode and cathode. The height of the cylindrical electrodes also can be important, as tall electrodes can promote inconsistent flow of fluids from bottom to top, as well as dissolved-oxygen gradients from bubbles generated. These factors can disrupt consistent homogeneity and uniform anode to cathode flow when comparing the bottom and top of the electrodes. Working electrodes can have a diameter of about 18 to about 25 cm, with heights not exceeding about 18 cm. Tilting the electrodes slightly may help offset the uneven-dissolved-oxygen effect. Inconsistent spacing between the electrodes can also be disruptive. Electrical current can be significantly higher between the closer-spaced surfaces of the electrodes, causing over-processing in these regions and robbing electrical current and proper reactions from surfaces that are farther apart. Inconsistencies in electrode spacing can also cause inconsistent and unpredictable results.

The amperage run through each electrode can be about 2 amps, about 3 amps, about 4 amps, about 5 amps, about 6 amps, about 7 amps, about 8 amps, about 9 amps, about 10 amps, about 11 amps, about 12 amps, about 13 amps, about 14 amps, or about 15 amps, between about 2 amps and about 15 amps, between about 4 amps and about 14 amps, at least about 2 amps, at least about 4 amps, at least about 6 amps, or any range created using any of these values. In one embodiment, 7 amps is used with each electrode.

The amperage can be run through the electrodes for a sufficient time to electrolyze the saline solution. Sufficient time can be about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, at least about 2 hr, at least about 3 hr, at least about 4 hr, at least about 5 hr, at least about 6 hr, at least about 7 hr, at least about 8 hr, at least about 9 hr, at least about 10 hr, at least about 11 hr, at least about 12 hr, at least about 13 hr, at least about 14 hr, at least about 15 hr, at least about 16 hr, at least about 17 hr, at least about 18 hr, at least about 19 hr, at least about 20 hr, at least about 21 hr, at least about 22 hr, at least about 23 hr, at least about 24 hr, between about 2 hr and about 8 hr, between about 3 hr and about 9 hr, between about 4 hr and about 10 hr, between about 5 hr and about 12 hr, between about 7 hr and about 9 hr, between about 6 hr and about 10 hr, between about 1 hr and about 10 hr, or between about 5 hr and about 15 hr.

The solution can be chilled during the electrochemical process. The temperature during this process can be about 0° C., about 1° C. about 2° C. about 3° C. about 4° C. about 5° C. about 6° C. about 7° C., about 8° C., about 9° C., about 10° C., about 11° C., about 12° C., between about 0° C. and about 10° C., between about 0° C. and about 5° C., between about 5° C. and about 10° C., between about 0° C. and about 7° C., or between about 2° C. and about 5° C. In one embodiment, the chilled solution can have a temperature of between about 4.5° C. and about 5.8° C.

The solution can also be mixed during the electrochemical process. This mixing can be performed to ensure substantially complete electrolysis. Again, a physical mixing apparatus can be used or a circulation or recirculation can be used. Circulation or recirculation can be at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. In one embodiment, circulation or recirculation can be at about 1,000 gal/hr.

The platinum surfaces on the electrodes by themselves can be optimal to catalyze the required reactions. Rough, double layered platinum plating can assure that local “reaction centers” (sharply pointed extrusions) are active and that the reactants not make contact with the underlying electrode titanium substrate. Tiny micropores in the platinum surface (caused by tiny bubbles in the platinum electroplating process) can allow the oxidative components to penetrate and oxidize the titanium base. This component penetration can degrade the electrodes and put undesirable titanium ions and oxides in the product. Double plated platinum can minimize the risk of micropores in the platinum surface going through to the titanium.

During electrolysis, oxygen and hydrogen bubbles themselves can form on the platinum surfaces during electrolysis and reduce the reactive surface area. Sharp, uneven surfaces can tend to minimize bubble adhesion and create stronger local electric fields, increasing efficiency.

Electric fields between the electrodes can cause movement of ions. Negative ions can move toward the anode and positive ions toward the cathode. This can enable necessary exchange of reactants and products between the electrodes. In some embodiments, no barriers are needed between the electrodes.

The configuration and electrical characteristics between the electrodes can be similar to conditions that exist between the mitochondrial membranes inside these cellular organelles. Inside living mitochondria, an electrical potential (voltage) is generated by the electron transport chain between the inner and outer mitochondrial membranes. This electrical potential is capable of causing electrolysis to take place in the mitochondria. There are many factors that regulate this voltage potential. This in principle is similar to the electrical potential maintained between the platinum electrode surfaces in the electrolysis cells. To further expand on the similarities, mitochondria produce superoxides by means of electron donation to dissolved oxygen in the cellular fluids. Similar electrochemistry can exist on the cathode of the platinum electrodes.

In the mitochondria, fluctuations of the mitochondrial potential, specifically pulsing of the potentials have been seen to take place. Pulsing potentials in the power supply of the production units can also be built in. Lack of filter capacitors in the rectified power supply can cause the voltages to drop to zero 120 times per second, resulting in a hard spike when the alternating current in the house power lines changes polarity. This hard spike, under Fourier transform, can emit a large bandwidth of frequencies. In essence, the voltage is varying from high potential to zero 120 times a second. In other embodiments, the voltage can vary from high potential to zero about 1,000 times a second, about 500 times a second, about 200 times a second, about 150 times a second, about 120 times a second, about 100 times a second, about 80 times a second, about 50 times a second, about 40 times a second, about 20 times a second, between about 200 times a second and about 20 times a second, between about 150 times a second and about 100 times a second, at least about 100 times a second, at least about 50 times a second, or at least about 120 times a second. This power modulation can allow the electrodes sample all voltages and also provides enough frequency bandwidth to excite resonances in the forming molecules themselves. The time at very low voltages can also provide an environment of low electric fields where ions of similar charge can come within close proximity to the electrodes. All of these factors together can provide a possibility for the formation of stable complexes capable of generating and preserving ROS free radicals.

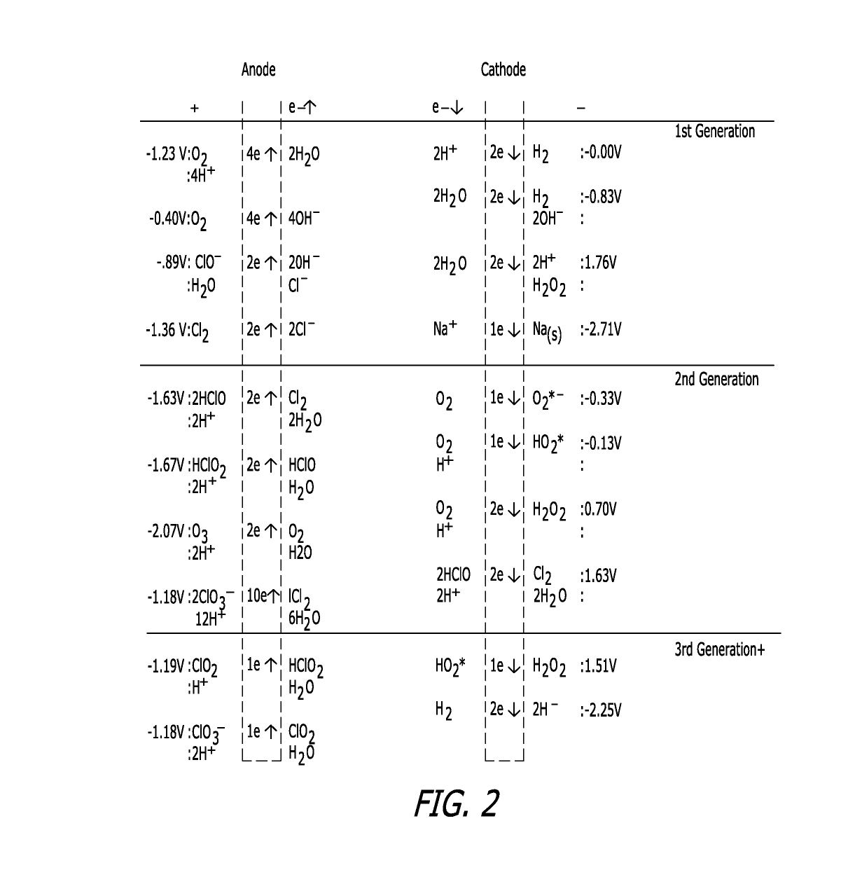

FIG. 2 illustrates an example diagram of the generation of various molecules at the electrodes, the molecules written between the electrodes depict the initial reactants and those on the outside of the electrodes depict the molecules/ions produced at the electrodes and their electrode potentials. The diagram is broken into generations where each generation relies on the products of the subsequent generations.

The end products of this electrolytic process can react within the saline solution to produce many different chemical entities. The compositions and beverage described herein can include one or more of these chemical entities. These end products can include, but are not limited to superoxides: O₂*⁻, HO₂*; hypochlorites: OCl⁻, HOCl, NaOCl; hypochlorates: HClO₂, ClO₂, HClO₃, HClO₄; oxygen derivatives: O₂, O₃, O₄*⁻, 1O; hydrogen derivatives: H₂, H⁻; hydrogen peroxide: H₂O₂; hydroxyl free Radical: OH*⁻; ionic compounds: Na⁺, Cl⁻, H⁺, OH⁻, NaCl, HCl, NaOH; chlorine: Cl₂; and water clusters: n*H₂O—induced dipolar layers around ions, several variations.

In order to determine the relative concentrations and rates of production of each of these during electrolysis, certain general chemical principles can be helpful:

1) A certain amount of Gibbs free energy is required for construction of the molecules; Gibbs free energy is proportional to the differences in electrode potentials listed in FIG. 2. Reactions with large energy requirements are less likely to happen, for example an electrode potential of −2.71V (compared to Hydrogen reduction at 0.00V) is required to make sodium metal:

Na⁺+1e⁻→Na₍ₛ₎

Such a large energy difference requirement makes this reaction less likely to happen compared to other reactions with smaller energy requirements. Electron(s) from the electrodes may be preferentially used in the reactions that require lesser amounts of energy, such as the production of hydrogen gas.

2) Electrons and reactants are required to be at the same micro-locality on the electrodes. Reactions that require several reactants may be less likely to happen, for example:

Cl₂+6H₂O→10e⁻+2ClO₃⁻+12H⁺

requires that 6 water molecules and a Cl₂ molecule to be at the electrode at the same point at the same time and a release of 10 electrons to simultaneously occur. The probability of this happening generally is smaller than other reactions requiring fewer and more concentrated reactants to coincide, but such a reaction may still occur.

3) Reactants generated in preceding generations can be transported or diffuse to the electrode where reactions happen. For example, dissolved oxygen (O₂) produced on the anode from the first generation can be transported to the cathode in order to produce superoxides and hydrogen peroxide in the second generation. Ions can be more readily transported: they can be pulled along by the electric field due to their electric charge. In order for chlorates, to be generated, for example, HClO₂ can first be produced to start the cascade, restrictions for HClO₂ production can also restrict any subsequent chlorate production. Lower temperatures can prevent HClO₂ production.

Stability and concentration of the above products can depend, in some cases substantially, on the surrounding environment. The formation of complexes and water clusters can affect the lifetime of the moieties, especially the free radicals.

In a pH-neutral aqueous solution (pH around 7.0) at room temperature, superoxide free radicals (O₂*⁻) have a half-life of 10's of milliseconds and dissolved ozone (O₃) has a half-life of about 20 min. Hydrogen peroxide (H₂O₂) is relatively long-lived in neutral aqueous environments, but this can depend on redox potentials and UV light. Other entities such as HCl and NaOH rely on acidic or basic environments, respectively, in order to survive. In pH-neutral solutions, H⁺ and OH⁻ ions have concentrations of approximately 1 part in 10,000,000 in the bulk aqueous solution away from the electrodes. H⁻ and ¹O can react quickly. The stability of most of these moieties mentioned above can depend on their microenvironment. Superoxides and ozone can form stable Van de Waals molecular complexes with hypochlorites. Clustering of polarized water clusters around charged ions can also have the effect of preserving hypochlorite-superoxide and hypochlorite-ozone complexes. Such complexes can be built through electrolysis on the molecular level on catalytic substrates, and may not occur spontaneously by mixing together components. Hypochlorites can also be produced spontaneously by the reaction of dissolved chlorine gas (Cl₂) and water. As such, in a neutral saline solution the formation of on or more of the stable molecules and complexes may exist: dissolved gases: O₂, H₂, Cl₂; hypochlorites: OCl⁻, HOCl, NaOCl; hypochlorates: HClO₂, ClO₂, HClO₃, HClO₄; hydrogen peroxide: H₂O₂; ions: Na⁺, Cl⁻, H⁺, H⁻, OH⁻; ozone: O₃, O₄*⁻; singlet oxygen: ¹O; hydroxyl free radical: OH*⁻; superoxide complexes: HOCl—O₂*⁻; and ozone complexes: HOCl—O₃. One or more of the above molecules can be found within the compositions and beverages described herein.

A complete quantum chemical theory can be helpful because production is complicated by the fact that different temperatures, electrode geometries, flows and ion transport mechanisms and electrical current modulations can materially change the relative/absolute concentrations of these components, which could result in producing different distinct compositions. As such, the selection of production parameters can be critical. The amount of time it would take to check all the variations experimentally may be prohibitive.

After amperage has been run through the solution for a sufficient time, an electrolyzed solution is created with beneficial properties, such as a life enhancing beverage. The solution can have a pH of about 7.4, about 7.5, about 7.6, about 7.7, about 7.8, about 7.9, about 8.0, about 8.1, about 8.2 about 8.3, about 8.4, between about 7.4 and about 8.4, or between about 8.0 and 8.2. In one embodiment, the pH is about 8.01. In some embodiments, the pH is greater than 7.4. In some embodiments, the pH is not acidic. In other embodiments, the solution can have a pH less than about 7.5. The pH may not be basic. The solution can be stored and or tested for particular properties in storage/testing step 112.

The chlorine concentration of the electrolyzed solution can be about 5 ppm, about 10 ppm, about 15 ppm, about 20 ppm, about 21 ppm, about 22 ppm, about 23 ppm, about 24 ppm, about 25 ppm, about 26 ppm, about 27 ppm, about 28 ppm, about 29 ppm, about 30 ppm, about 31 ppm, about 32 ppm, about 33 ppm, about 34 ppm, about 35 ppm, about 36 ppm, about 37 ppm, about 38 ppm, less than about 38 ppm, less than about 35 ppm, less than about 32 ppm, less than about 28 ppm, less than about 24 ppm, less than about 20 ppm, less than about 16 ppm, less than about 12 ppm, less than about 5 ppm, between about 30 ppm and about 34 ppm, between about 28 ppm and about 36 ppm, between about 26 ppm and about 38 ppm, between about 20 ppm and about 38 ppm, between about 5 ppm and about 34 ppm, between about 10 ppm and about 34 ppm, or between about 15 ppm and about 34 ppm. In one embodiment, the chlorine concentration is about 32 ppm. In another embodiment, the chlorine concentration is less than about 41 ppm.

The saline concentration in the electrolyzed solution can be about 0.10% w/v, about 0.11% w/v, about 0.12% w/v, about 0.13% w/v, about 0.14% w/v, about 0.15% w/v, about 0.16% w/v, about 0.17% w/v, about 0.18% w/v, about 0.19% w/v, about 0.20% w/v, about 0.30% w/v, about 0.40% w/v, about 0.50% w/v, about 0.60% w/v, about 0.70% w/v, between about 0.10% w/v and about 0.20% w/v, between about 0.11% w/v and about 0.19% w/v, between about 0.12% w/v and about 0.18% w/v, between about 0.13% w/v and about 0.17% w/v, or between about 0.14% w/v and about 0.16% w/v.

The beverage generally can include electrolytic and/or catalytic products of pure saline that mimic redox signaling molecular compositions of the native salt water compounds found in and around human cells. The beverage can be fine tuned to mimic or mirror molecular compositions of different biological media. The life enhancing beverage can have reactive species other than chlorine present. As described, species present in the compositions and beverages described herein can include, but are not limited to O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, ¹O, OH*⁻, HOCl—O₂*⁻, HOCl—O₃, O₂*⁻, HO₂*, NaCl, HCl, NaOH, and water clusters: n*H₂O—induced dipolar layers around ions, several variations.

Depending on the parameters used to produce the beverage, different components can be present at different concentrations. In one embodiment, the beverage can include about 0.1 ppt, about 0.5 ppt, about 1 ppt, about 1.5 ppt, about 2 ppt, about 2.5 ppt, about 3 ppt, about 3.5 ppt, about 4 ppt, about 4.5 ppt, about 5 ppt, about 6 ppt, about 7 ppt, about 8 ppt, about 9 ppt, about 10 ppt, about 20 ppt, about 50 ppt, about 100 ppt, about 200 ppt, about 400 ppt, about 1,000 ppt, between about 0.1 ppt and about 1,000 ppt, between about 0.1 ppt and about 100 ppt, between about 0.1 ppt and about 10 ppt, between about 2 ppt and about 4 ppt, at least about 0.1 ppt, at least about 2 ppt, at least about 3 ppt, at most about 10 ppt, or at most about 100 ppt of OCl⁻. In some embodiments, OCI⁻ can be present at about 3 ppt. In other embodiments, OCI⁻ can be the predominant chlorine containing species in the beverage.

In some embodiments, hydroxyl radicals can be stabilized in the beverage by the formation of radical complexes. The radical complexes can be held together by hydrogen bonding. Another radical that can be present in the beverage is an OOH. radical. Still other radical complexes can include a nitroxyl-peroxide radical (HNO—HOO*) and/or a hypochlorite-peroxide radical (HOCl—HOO*).

Reactive species' concentrations in the life enhancing solutions, detected by fluorescence photo spectroscopy, may not significantly decrease in time. Mathematical models show that bound HOCl—*O₂⁻ complexes are possible at room temperature. Molecular complexes can preserve volatile components of reactive species. For example, reactive species concentrations in whole blood as a result of molecular complexes may prevent reactive species degradation over time.

Reactive species can be further divided into “reduced species” (RS) and “reactive oxygen species” (ROS). Reactive species can be formed from water molecules and sodium chloride ions when restructured through a process of forced electron donation. Electrons from lower molecular energy configurations in the salinated water may be forced into higher, more reactive molecular configurations. The species from which the electron was taken can be “electron hungry” and is called the RS and can readily become an electron acceptor (or proton donor) under the right conditions. The species that obtains the high-energy electron can be an electron donor and is called the ROS and may energetically release these electrons under the right conditions.

When an energetic electron in ROS is unpaired it is called a “radical”. ROS and RS can recombine to neutralize each other by the use of a catalytic enzyme. Three elements, (1) enzymes, (2) electron acceptors, and (3) electron donors can all be present at the same time and location for neutralization to occur.

In some embodiments, substantially no organic material is present in the beverages described. Substantially no organic material can be less than about 0.1 ppt, less than about 0.01 ppt, less than about 0.001 ppt or less than about 0.0001 ppt of total organic material.

The life enhancing beverage can be stored and bottled as needed to ship to consumers. The life enhancing beverage can have a shelf life of about 5 days, about 30 days, about 3 months, about 6 months, about 9 months, about 1 year, about 1.5 years, about 2 years, about 3 years, about 5 years, about 10 years, at least about 5 days, at least about 30 days, at least about 3 months, at least about 6 months, at least about 9 months, at least about 1 year, at least about 1.5 years, at least about 2 years, at least about 3 years, at least about 5 years, at least about 10 years, between about 5 days and about 1 year, between about 5 days and about 2 years, between about 1 year and about 5 years, between about 90 days and about 3 years, between about 90 days and about 5 year, or between about 1 year and about 3 years.

The life enhancing beverage can then be bottled in a bottling step 114. The beverage can be bottled in plastic bottles having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values. The plastic bottles can also be plastic squeezable pouches having similar volumes. In one embodiment, plastic squeezable pouches can have one way valves to prevent leakage of the life enhancing beverage, for example, during athletic activity.

During bottling, solution from an approved batch can be pumped through a 10 micron filter (e.g., polypropylene) to remove any larger particles from tanks, dust, hair, etc. that might have found their way into the batch. In other embodiments, this filter need not be used. Then, the solution can be pumped into the bottles, the overflow going back into the batch.

Bottles generally may not contain any dyes, metal specks or chemicals that can be dissolved by acids or oxidating agents. The bottles, caps, bottling filters, valves, lines and heads used can be specifically be rated for acids and oxidating agents. Caps and with organic glues, seals or other components sensitive to oxidation may be avoided, as these could neutralize and weaken the product over time.

The bottles and pouches used herein can aid in preventing decay of free radical species found within the beverages. In other embodiments, the bottles and pouches described do not further the decay process. In other words, the bottles and pouches used can be inert with respect to the radical species in the beverages. In one embodiment, a container (e.g., bottle and/or pouch) can allow less than about 10% decay/month, less than about 9% decay/month, less than about 8% decay/month, less than about 7% decay/month, less than about 6% decay/month, less than about 5% decay/month, less than about 4% decay/month, less than about 3% decay/month, less than about 2% decay/month, less than about 1% decay/month, between about 10% decay/month and about 1% decay/month, between about 5% decay/month and about 1% decay/month, about 10% decay/month, about 9% decay/month, about 8% decay/month, about 7% decay/month, about 6% decay/month, about 5% decay/month, about 4% decay/month, about 3% decay/month, about 2% decay/month, or about 1% decay/month of free radicals in the beverage. In one embodiment, a bottle can only result in about 3% decay/month of superoxide. In another embodiment, a pouch can only result in about 4% decay/month of superoxide.

Quality Assurance testing can be done on every batch before the batch can be approved for bottling or can be performed during or after bottling. A 16 oz. sample bottle can be taken from each complete batch and analyzed. Determinations for presence of contaminants such as heavy metals or chlorates can be performed. Then pH, Free and Total Chlorine concentrations and reactive molecule concentrations of the active ingredients can be analyzed by fluorospectroscopy methods. These results can be compared to those of a standard solution which is also tested along side every sample. If the results for the batch fall within a certain range relative to the standard solution, it can be approved. A chemical chromospectroscopic MS analysis can also be run on random samples to determine if contaminants from the production process are present.

The beverage can be consumed by ingestion. In other embodiments, the beverage can be provided as a solution for injection. In some embodiments, injection can be subcutaneous, intra-luminal, site specific (e.g., injected into a cancer or internal lesion), or intramuscular. Intravenous injection can also be desirable. The life enhancing solution can be packaged in plastic medical solution pouches having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values, and these pouches can be used with common intravenous administration systems.

Flavors can be added to the life enhancing beverages. Flavor additives introduced into the life enhancing beverages may not substantially degrade any of the beneficial components of the beverage. In one embodiment, a flavor does not substantially degrade more than about 5%, more than about 4%, more than about 3%, more than about 2%, more than about 1%, more than about 0.5%, more than about 0.1%, more than about 0.05%, more than about 0.01%, more than about 0.005%, more than about 0.001%, more than about 0.0005%, or more than about 0.0001% of the life enhancing beverage. Flavors can include chocolate, fruit flavors, coffee flavor, mint, and the like.

When administered as a liquid beverage, it can be taken once, twice, three times, four times or more a day. Each administration can be about 1 oz, about 2 oz, about 3 oz, about 4 oz, about 5 oz, about 6 oz, about 7 oz, about 8 oz, about 9 oz, about 10 oz, about 11 oz, about 12 oz, about 16 oz, about 20 oz, about 24 oz, about 28 oz, about 32 oz, about 34 oz, about 36 oz, about 38 oz, about 40 oz, about 46 oz, between about 1 oz and about 32 oz, between about 1 oz and about 16 oz, between about 1 oz and about 8 oz, at least about 2 oz, at least about 4 oz, or at least about 8 oz. In one embodiment, the beverage can be administered at a rate of about 4 oz twice a day.

In other embodiments, the administration can be acute or long term. For example, the beverage can be consumed for a day, a week, a month, a year or longer. In other embodiments, the beverage can simply be taken as needed such as for exercise.

The beverages described herein when administered can be used to treat a condition or a disease or can enhance a life condition or a condition associated with a disease. For example, when administered alongside exercise, the beverages described herein can increase the density of mitochondrial DNA. For example, an increase in mitochondrial DNA of about 1%, about 5%, about 10%, about 15%, about 20%, about 21%, about 22%, about 23%, about 24%, about 25%, about 26%, about 27%, about 28%, about 29%, about 30%, about 32%, about 34%, about 36%, about 38%, about 40%, about 45%, between about 1% and about 40%, between about 1% and about 10%, between about 20% and about 30%, at least about 5%, at least about 10%, or at least about 20% when compared to an individual who has not taken the beverage. An increase in mitochondrial DNA can result in a lower level of free radicals in the blood which can in turn lead to a reduced amount of oxidative stress.

An increase in mitochondrial DNA can be used to treat a condition or a disease or can enhance a life condition or a condition associated with a disease. As such, the beverages described can treat conditions or diseases such as, but not limited to sacropenia, Parkinson's disease, neuro-related age disease, obesity, aging, life stresses such as those caused by fear, neurodegenerative diseases, cognitive disorders, obesity, reduced metabolic rate, metabolic syndrome, diabetes mellitus, cardiovascular disease, hyperlipidemia, neurodegenerative disease, cognitive disorder, mood disorder, stress, and anxiety disorder; for weight management, or to increase muscle performance or mental performance, AIDS, dementia complex, Alzheimer's disease, amyotrophic lateral sclerosis, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Huntington's disease, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome, cognitive function abnormalities, perception abnormalities, attention disorders, speech comprehension disorders, reading comprehension disorders, creation of imagery disorders, learning disorders, reasoning disorders, mood disorders, depression, postpartum depression, dysthymia, bipolar disorder, generalized anxiety disorder, panic disorder, panic disorder with agoraphobia, agoraphobia, social anxiety disorder, obsessive-compulsive disorder, post-traumatic stress disorder, musculoskeletal disorder, lack of strength, lack of endurance, cancer, atherosclerotic lesions, atherosclerosis, oxidative stress, atherogenesis, hypertension, hypercholesterolemia, and degenerative diseases.

The beverages described herein when administered can be used to increase athletic performance. The beverages can increase athletic performance by releasing free fatty acids into the blood stream to help fuel active muscles. For example, when taken before or concurrently with exercise, one can increase their time to exhaustion. For example, when using the beverage, one can increase their time to exhaustion by about 1%, about 5%, about 10%, about 15%, about 20%, about 21%, about 22%, about 23%, about 24%, about 25%, about 26%, about 27%, about 28%, about 29%, about 30%, about 32%, about 34%, about 36%, about 38%, about 40%, about 45%, between about 1% and about 40%, between about 1% and about 10%, between about 20% and about 30%, at least about 5%, at least about 10%, or at least about 20% when compared to an individual who has not taken the beverage.

Also, the beverage can increase a recipient's VO₂ₘₐₓ. The beverage can contain signaling molecules. Within 30 minutes of drinking, small molecules form, shifting the metabolome. Athletes drinking the beverage for one week or longer can experience a shift in up to 43 metabolites, such as free fatty acids and energy intermediates. People, and even animals, treated with ASEA for one week ran 29% longer until exhausted. Muscle and liver glycogen can be modulated by administration of ASEA. Muscle fatty acid β-oxidation can be modulated by administration of ASEA. Muscle carbonyls can be modulated by administration of ASEA.

The beverage can contain signaling molecules. Within 30 minutes of drinking, small molecules can form thereby shifting a users metabolome. Athletes drinking the beverage for one week or longer can experience a shift in up to 43 metabolites, such as free fatty acids and energy intermediates.

People, and even animals, treated with ASEA for one week ran on average about 29% longer until exhausted. In other embodiments, those treated with ASEA for one week ran on average about 1%, about 5%, about 10%, about 15%, about 20%, about 21%, about 22%, about 23%, about 24%, about 25%, about 26%, about 27%, about 28%, about 29%, about 30%, about 32%, about 34%, about 36%, about 38%, about 40%, about 45%, between about 1% and about 40%, between about 1% and about 10%, between about 20% and about 30%, at least about 5%, at least about 10%, or at least about 20% longer until exhaustion.

Muscle and liver glycogen can be modulated by administration of ASEA. For example, the rate of muscle glycogen depletion can be reduced by about 1%, about 5%, about 10%, about 15%, about 20%, about 21%, about 22%, about 23%, about 24%, about 25%, about 26%, about 27%, about 28%, about 29%, about 30%, about 32%, about 33% about 34%, about 36%, about 38%, about 40%, about 45%, between about 1% and about 40%, between about 1% and about 10%, between about 20% and about 30%, at least about 5%, at least about 10%, or at least about 20% longer when compared to those not treated. In one embodiment, administration of ASEA in conjunction with exercise can reduce the rate of muscle glycogen depletion by about 33%.

Muscle fatty acid 13-oxidation can be modulated by administration of ASEA. Muscle carbonyls can be modulated by administration of ASEA.

### Example 1

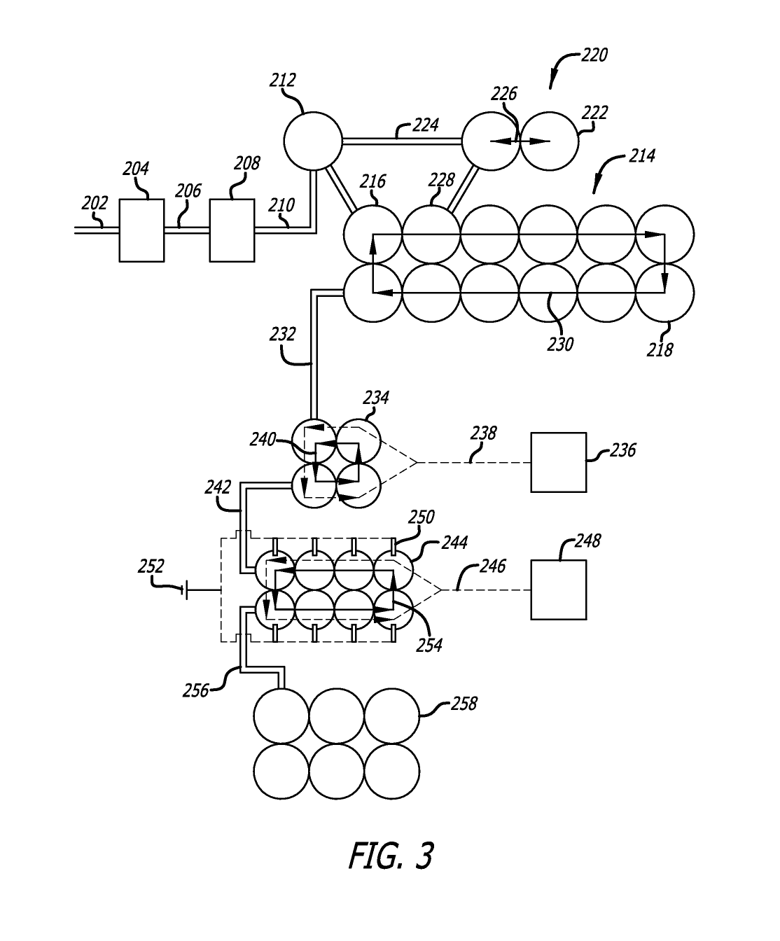

FIG. 3 illustrates a plan view of a process and system for producing a life enhancing beverage according to the present description. One skilled in the art understands that changes can be made to the system to alter the life enhancing beverage, and these changes are within the scope of the present description.

Incoming water 202 can be subjected to reverse osmosis system 204 at a temperature of about 15-20° C. to achieve purified water 206 with about 8 ppm of total dissolves solids. Purified water 206, is then fed at a temperature of about 15-20° C. into distiller 208 and processed to achieve distilled water 210 with about 0.5 ppm of total dissolved solids. Distilled water 210 can then be stored in tank 212.

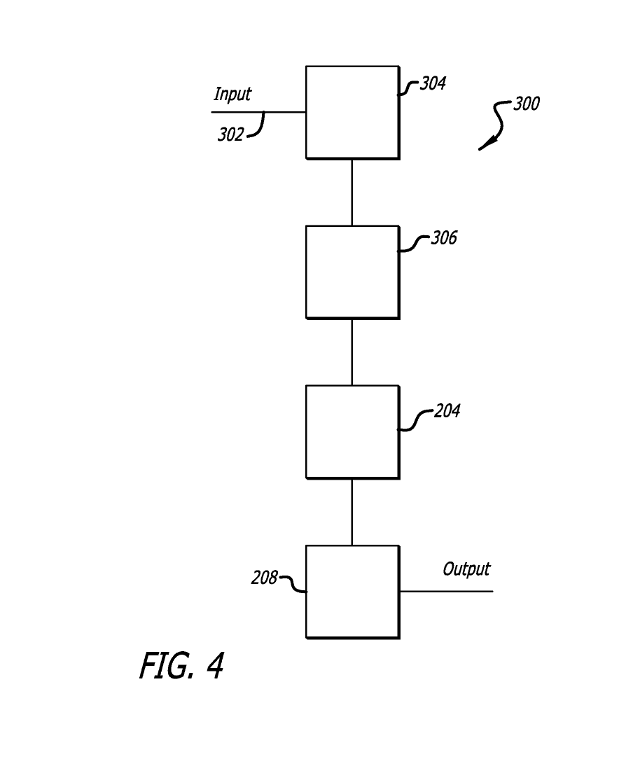

FIG. 4 illustrates an example system for preparing water for further processing into a life enhancing beverage. System 300 can include a water source 302 which can feed directly into a carbon filter 304. After oils, alcohols, and other volatile chemical residuals and particulates are removed by carbon filter 304, the water can be directed to resin beds within a water softener 306 which can remove dissolved minerals. Then, as described above, the water can pass through reverse osmosis system 204 and distiller 208.

As needed, distilled water 210 can be gravity fed from tank 212 into saline storage tank cluster 214 using line 216. Saline storage tank cluster 214 in one embodiment can include twelve tanks 218. Each tank 218 can be filled to about 1,300 gallons with distilled water 210. A handheld meter can be used to test distilled water 210 for salinity.

Saline storage tank cluster 214 is then salted using a brine system 220. Brine system 220 can include two brine tanks 222. Each tank can have a capacity of about 500 gallons. Brine tanks 222 are filled to 475 gallons with distilled water 210 using line 224 and then NaCl is added to the brine tanks 222 at a ratio of about 537.5 g/gal of liquid. At this point, the water is circulated 226 in the brine tanks 222 at a rate of about 2,000 gal/hr for about 4 days.

Prior to addition of brine to tanks 218, the salinity of the water in tanks 218 can be tested using a handheld conductivity meter such as an YSI ECOSENSE® ecp300 (YSI Inc., Yellow Springs, Ohio). Any corrections based on the salinity measurements can be made at this point. Brine solution 228 is then added to tanks 218 to achieve a salt concentration of about 10.75 g/gal. The salted water is circulated 230 in tanks 218 at a rate of about 2,000 gal/hr for no less than about 72 hr. This circulation is performed at room temperature. A handheld probe can again be used to test salinity of the salinated solution. In one embodiment, the salinity is about 2.8 ppth.

In one example method for filling and mixing the salt water in the brine holding tanks, the amount of liquid remaining in the tanks is measured. The amount of liquid remaining in a tank is measured by recording the height that the liquid level is from the floor that sustains the tank, in centimeters, and referencing the number of gallons this height represents. This can be done from the outside of the tank if the tank is semi-transparent. The initial liquid height in both tied tanks can also be measured. Then, after ensuring that the output valve is closed, distilled water can be pumped in. The amount of distilled water that is being pumped into a holding tank can then be calculated by measuring the rise in liquid level: subtracting the initial height from the filled height and then multiplying this difference by a known factor.

The amount of salt to be added to the tank is then calculated by multiplying 11 grams of salt for every Gallon of distilled water that has been added to the tank. The salt can be carefully weighed out and dumped into the tank.

The tank is then agitated by turning on the recirculation pump and then opening the top and bottom valves on the tank. Liquid is pumped from the bottom of the tank to the top. The tank can be agitated for three days before it may be ready to be processed.

After agitating the tank for more than 6 hours, the salinity is checked with a salinity meter by taking a sample from the tank and testing it. Salt or water can be added to adjust the salinity within the tanks. If either more water or more salt is added then the tanks are agitated for 6 more hours and tested again. After about three days of agitation, the tank is ready to be processed.

Salinated water 232 is then transferred to cold saline tanks 234. In one embodiment, four 250 gal tanks are used. The amount of salinated water 232 moved is about 1,000 gal. A chiller 236 such as a 16 ton chiller is used to cool heat exchangers 238 to about 0-5° C. The salinated water is circulated 240 through the heat exchangers which are circulated with propylene glycol until the temperature of the salinated water is about 4.5-5.8° C. Chilling the 1,000 gal of salinated water generally takes about 6-8 hr.

Cold salinated water 242 is then transferred to processing tanks 244. In one embodiment, eight tanks are used and each can have a capacity of about 180 gal. Each processing tank 244 is filled to about 125 gal for a total of 1,000 gal. Heat exchangers 246 are again used to chill the cold salinated water 242 added to processing tanks 244. Each processing tank can include a cylinder of chilling tubes and propylene glycol can be circulated. The heat exchangers can be powered by a 4-5 ton chiller 248. The temperature of cold salinated water 242 can remain at 4.5-5.8° C. during processing.

Prior to transferring aged salt water to processing tanks, the aged salt water can be agitated for about 30 minutes to sufficiently mix the aged salt water. Then, the recirculation valves can then be closed, the appropriate inlet valve on the production tank is opened, and the tank filled so that the salt water covers the cooling coils and comes up to the fill mark (approximately 125 gallons).

Once the aged salt water has reached production temperature, turn off the recirculation pump but leave the chiller on. The tank should be adequately agitated or re-circulated during the whole duration of electrochemical processing and the temperature should remain constant throughout.

Each processing tank 244 includes electrode 250. Electrodes 250 can be 3 inches tall circular structures formed of titanium and plated with platinum. Electrochemical processing of the cold salinated water can be run for 8 hr. A power supply 252 is used to power the eight electrodes (one in each processing tank 244) to 7 amps each for a total of 56 amps. The cold salinated water is circulated 254 during electrochemical processing at a rate of about 1,000 gal/hr.

An independent current meter can be used to set the current to around 7.0 Amps. Attention can be paid to ensure that the voltage does not exceed 12V and does not go lower than 9V. Normal operation can be about 10V.

A run timer can be set for a prescribed time (about 4.5 to 5 hours). Each production tank can have its own timer and/or power supply. Electrodes should be turned off after the timer has expired.

The production tanks can be checked periodically. The temperature and/or electrical current can be kept substantially constant. At the beginning, the electrodes can be visible from the top, emitting visible bubbles. After about 3 hours, small bubbles of un-dissolved oxygen can start building up in the tank as oxygen saturation occurs, obscuring the view of the electrodes. A slight chlorine smell can be normal.

After the 8 hour electrochemical processing is complete, life enhancing water 256 has been created with a pH of about 7.4, 32 ppm of chlorine, 100% OCl⁻ and 100% O⁻². Life enhancing water 256 is transferred to storage tanks 258 where the life enhancing water awaits bottling where it is shipped to consumers as a life enhancing beverage.

### Example 2

### Characterization of a Beverage Produced as Described in Example 1

A beverage produced as described in Example 1 and marketed under the trade name ASEA® was analyzed using a variety of different characterization techniques. ICP/MS and 35Cl NMR were used to analyze and quantify chlorine content. Headspace mass spectrometry analysis was used to analyze adsorbed gas content in the beverage. 1H NMR was used to verify the organic matter content in the beverage. 31P NMR and EPR experiments utilizing spin trap molecules were used to explore the beverage for free radicals.

The beverage was received and stored at about 4° C. when not being used.

### Chlorine NMR

Sodium hypochlorite solutions were prepared at different pH values. 5% sodium hypochlorite solution had a pH of 12.48. Concentrated nitric acid was added to 5% sodium hypochlorite solution to create solutions that were at pH of 9.99, 6.99, 5.32, and 3.28. These solutions were then analyzed by NMR spectroscopy. The beverage had a measured pH=8.01 and was analyzed directly by NMR with no dilutions.

NMR spectroscopy experiments were performed using a 400 MHz Bruker spectrometer equipped with a BBO probe. Cl35 NMR experiments were performed at a frequency of 39.2 MHz using single pulse experiments. A recycle delay of 10 seconds was used, and 128 scans were acquired per sample. A solution of NaCl in water was used as an external chemical shift reference. All experiments were performed at room temperature.

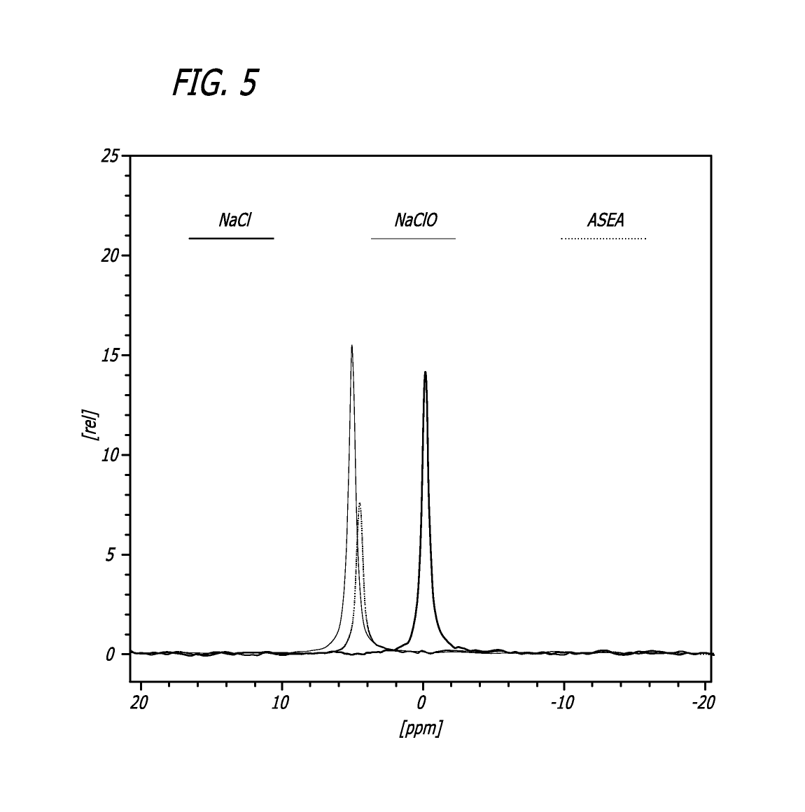

Cl35 NMR spectra were collected for NaCl solution, NaClO solutions adjusted to different pH values, and the beverage. FIG. 5 shows the Cl35 spectra of NaCl, NaClO solution at a pH of 12.48, and the beverage. The chemical shift scale was referenced by setting the Cl⁻ peak to 0 ppm. NaClO solutions above a pH=7 had identical spectra with a peak at approximately 5.1 ppm. Below pH of 7.0, the ClO⁻ peak disappeared and was replaced by much broader, less easily identifiable peaks. The beverage was presented with one peak at approximately 4.7 ppm, from clO⁻ in the beverage solution. This peak was integrated to estimate the concentration of clO⁻ in the beverage solution, which was determined to be 2.99 ppt or 0.17 M of ClO⁻ in the beverage.

### Proton NMR

An ASEA sample was prepared by adding 550 μL of ASEA and 50 μL of D₂O (Cambridge Isotope Laboratories) to an NMR tube and vortexing the sample for 10 seconds. ¹H NMR experiments were performed on a 700 MHz Bruker spectrometer equipped with a QNP cryogenically cooled probe. Experiments used a single pulse with pre-saturation on the water resonance experiment. A total of 1024 scans were taken. All experiments were performed at room temperature.

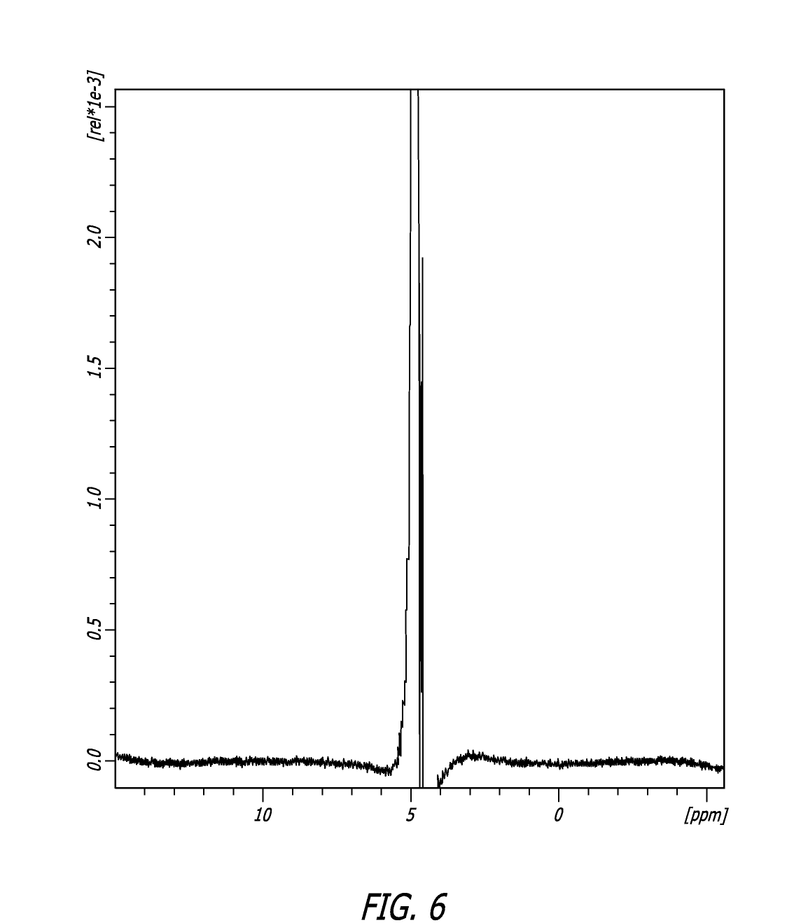

A ¹H NMR spectrum of the beverage was collected and is presented in FIG. 6. Only peaks associated with water were able to be distinguished from this spectrum. This spectrum show that very little if any organic material can be detected in the beverage using this method.

### Phosphorous NMR and Mass Spectrometry

DIPPMPO (5-(Diisopropoxyphosphoryl)-5-1-pyrroline-N-oxide) (VWR) samples were prepared by measuring about 5 mg of DIPPMPO into a 2 mL centrifuge tube. This tube then had 550 μL of either the beverage or water added to it, followed by 50 μL of O₂O. A solution was also prepared with the beverage but without DIPPMPO. These solutions were vortexed and transferred to NMR tubes for analysis. Samples for mass spectrometry analysis were prepared by dissolving about 5 mg of DIPPMPO in 600 μL of the beverage and vortexing, then diluting the sample by adding 100 μL of sample and 900 μL of water to a vial and vortexing.

NMR experiments were performed using a 700 MHz Bruker spectrometer equipped with a QNP cryogenically cooled probe. Experiments performed were a single 30o pulse at a P31 frequency of 283.4 MHz. A recycle delay of 2.5 seconds and 16384 scans were used. Phosphoric acid was used as an external standard. All experiments were performed at room temperature.

Mass spectrometry experiments were performed by directly injecting the ASEA/DIPPMPO sample into a Waters/Synapt Time of Flight mass spectrometer. The sample was directly injected into the mass spectrometer, bypassing the LC, and monitored in both positive and negative ion mode.

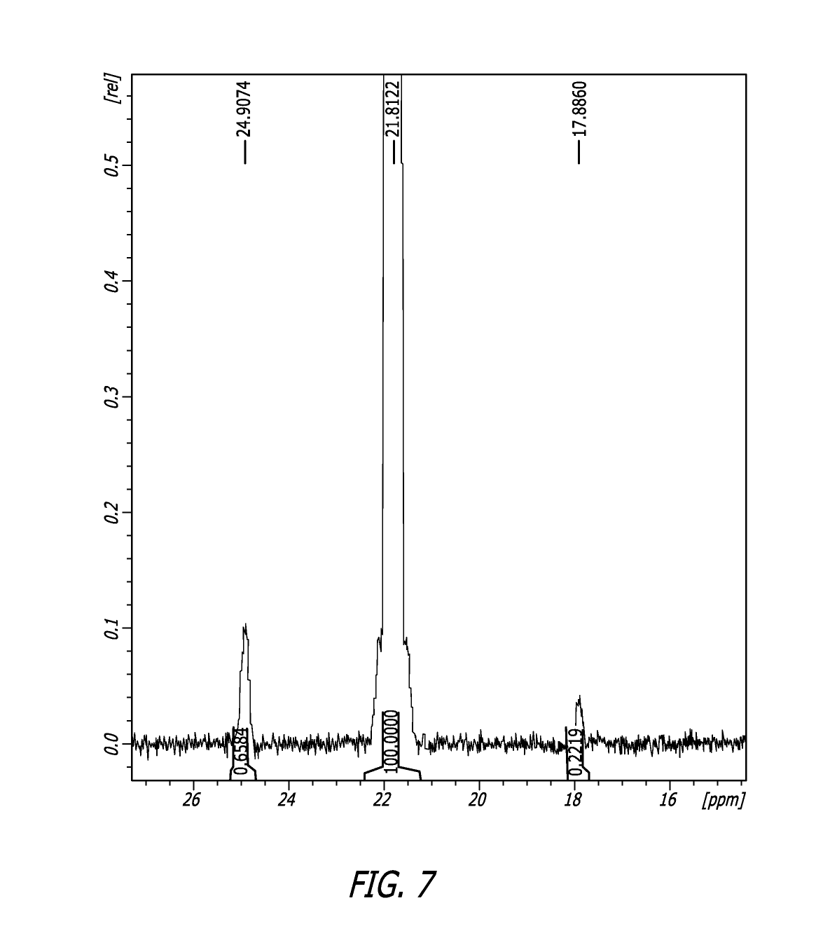

P31 NMR spectra were collected for DIPPMPO in water, the beverage alone, and the beverage with DIPPMPO added to it. An external reference of phosphoric acid was used as a chemical shift reference. FIG. 7 shows the 31P NMR spectrum of DIPPMPO combined with the beverage. The peak at 21.8 ppm was determined to be DIPPMPO and is seen in both the spectrum of DIPPMPO with the beverage (FIG. 7) and without the beverage (not pictured). The peak at 24.9 ppm is most probably DIPPMPO/OH. as determined in other DIPPMPO studies. This peak may be seen in DIPPMPO mixtures both with and without the beverage, but is detected at a much greater concentration in the solution with the beverage. In the DIPPMPO mixture with the beverage, there is another peak at 17.9 ppm. This peak is believed to be from another radical species in the beverage solution. This radical species may be OOH. or possibly a different radical complex. The approximate concentrations of spin trap complexes in the beverage/DIPPMPO solution are as follows:

| Solution | Concentration | |
|---|---|---|
| DIPPMPO | 36.6 | mM |
| DIPPMPO/OH• | 241 | μM |
| DIPPMPO/radical | 94 | μM |

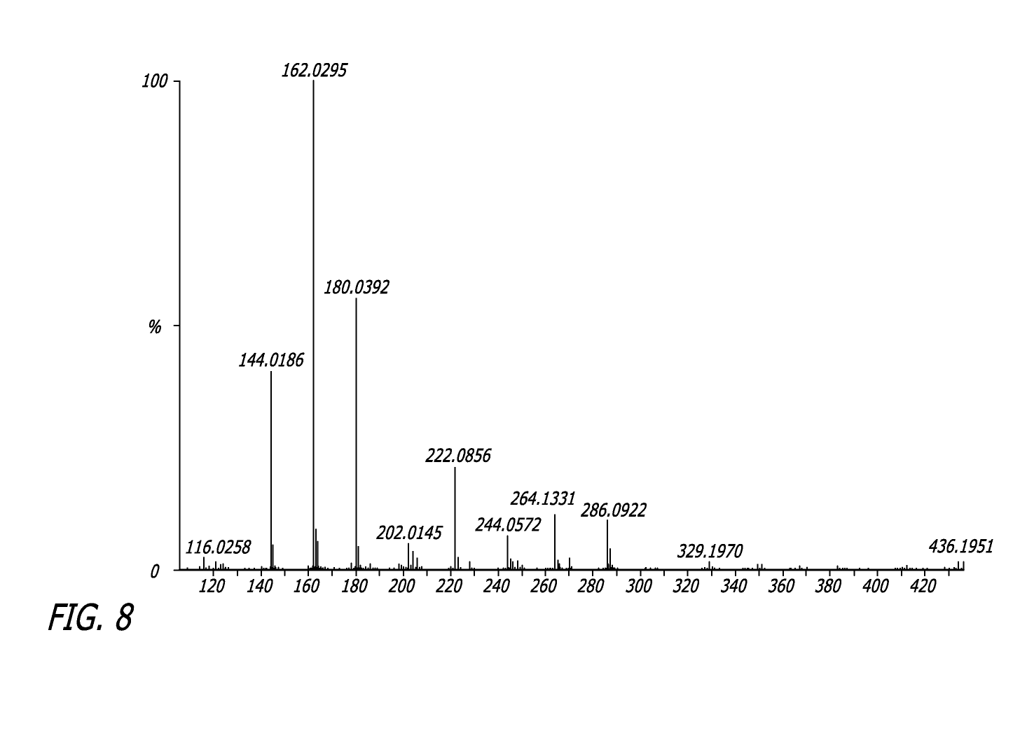

Mass spectral data was collected in an attempt to determine the composition of the unidentified radical species. The mass spectrum shows a parent peak and fragmentation pattern for DIPPMPO with m/z peaks at 264, 222, and 180, as seen in FIG. 8. FIG. 8 also shows peaks for the DIPPMPO/Na adduct and subsequent fragments at 286, 244, and 202 m/z. Finally, FIG. 8 demonstrates peaks for one DIPPMPO/radical complex with m/z of 329. The negative ion mode mass spectrum also had a corresponding peak at m/z of 327. There are additional peaks at 349, 367, and 302 at a lower intensity as presented in FIG. 8. None of these peaks could be positively confirmed. However, there are possible structures that would result in these mass patterns. One possibility for the peak generated at 329 could be a structure formed from a radical combining with DIPPMPO. Possibilities of this radical species include a nitroxyl-peroxide radical (HNO—HOO.) that may have formed in the beverage as a result of reaction with nitrogen from the air. Another peak at 349 could also be a result of a DIPPMPO/radical combination. Here, a possibility for the radical may be hypochlorite-peroxide (HOCl⁻HOO.). However, the small intensity of this peak and small intensity of the corresponding peak of 347 in the negative ion mode mass spectrum indicate this could be a very low concentration impurity and not a compound present in the ASEA solution.

### ICP/MS Analysis

Samples were analyzed on an Agilent 7500 series inductively-coupled plasma mass spectrometer (ICP-MS) in order to confirm the hypochlorite concentration that was determined by NMR. A stock solution of 5% sodium hypochlorite was used to prepare a series of dilutions consisting of 300 ppb, 150 ppb, 75 ppb, 37.5 ppb, 18.75 ppb, 9.375 ppb, 4.6875 ppb, 2.34375 ppb, and 1.171875 ppb in deionized Milli-Q water. These standards were used to establish a standard curve.

Based on NMR hypochlorite concentration data, a series of dilutions was prepared consisting of 164.9835 ppb, 82.49175 ppb, 41.245875 ppb, 20.622937 ppb, 10.311468 ppb, and 5.155734 ppb. These theoretical values were then compared with the values determined by ICP-MS analysis. The instrument parameters were as follows:

| Elements analyzed | 35 Cl, 37 Cl | |
|---|---|---|
| # of points per mass | 20 | |
| # of repetitions | 5 | |
| Total acquisition time | 68.8 | s |
| Uptake speed | 0.50 | rps |
| Uptake time | 33 | s |
| Stabilization time | 40 | s |
| Tune | No Gas | |
| Nebulizer flow rate | 1 | mL/min |
| Torch power | 1500 | W |

The results of the ICP-MS analysis are as follows:

| | Measured | Concentration |
|---|---|---|
| Dilution | Concentration (ppb) | by NMR (ppb) |
| 1 | 81 | 82 |
| 2 | 28 | 41 |
| 3 | 24 | 21 |
| 4 | 13 | 10 |
| 5 | 8 | 5 |

Dilutions were compared graphically to the ICP-MS signals and fit to a linear equation (R²=0.9522). Assuming linear behavior of the ICP-MS signal, the concentration of hypochlorite in the beverage was measured to be 3.02 ppt. Concentration values were determined by calculating the concentration of dilutions of the initial beverage and estimating the initial beverage hypochlorite concentration to be 3 ppt (as determined from 35Cl NMR analysis). The ICP-MS data correlate well with the 35Cl NMR data, confirming a hypochlorite concentration of roughly ⅓% (3 ppt). It should be noted that ICP-MS analysis is capable of measuring total chlorine atom concentration in solution, but not specific chlorine species. The NMR data indicate that chlorine predominantly exists as clO⁻ in the beverage.

### Gas Phase Quadrupole MS

### Sample Prep

Three sample groups were prepared in triplicate for the analysis: 1) Milli-Q deionized water 2) the beverage, and 3) 5% sodium hypochlorite standard solution. The vials used were 20 mL headspace vials with magnetic crimp caps (GERSTEL). A small stir bar was placed in each vial (VWR) along with 10 mL of sample. The vials were capped, and then placed in a Branson model 5510 sonicator for one hour at 60° C.

The sonicator was set to degas which allowed for any dissolved gasses to be released from the sample into the headspace. After degassing, the samples were placed on a CTC PAL autosampler equipped with a heated agitator and headspace syringe. The agitator was set to 750 rpm and 95° C. and the syringe was set to 75° C. Each vial was placed in the agitator for 20 min prior to injection into the instrument. A headspace volume of 2.5 mL was collected from the vial and injected into the instrument.

### Instrument Parameters

The instrument used was an Agilent 7890A GC system coupled to an Agilent 5975C El/Cl single quadrupole mass selective detector (MSD) set up for electron ionization. The GC oven was set to 40° C. with the front inlet and the transfer lines being set to 150° C. and 155° C. respectively. The carrier gas used was helium and it was set to a pressure of 15 PSI.

The MSD was set to single ion mode (SIM) in order to detect the following analytes:

| Analyte | Mass |
|---|---|
| Water | 18 |
| Nitrogen | 28 |
| Oxygen | 32 |
| Argon | 40 |
| Carbon Dioxide | 44 |
| Chlorine | 70 |
| Ozone | 48 |

The ionization source temperature was set to 230° C. and the quadrupole temperature was set to 150° C. The electron energy was set to 15 V.

Mass spectrometry data was obtained from analysis of the gas phase headspace of the water, the beverage, and hypochlorite solution. The raw area counts obtained from the mass spectrometer were normalized to the area counts of nitrogen in order to eliminate any systematic instrument variation. Both nitrogen and water were used as standards because they were present in equal volumes in the vial with nitrogen occupying the headspace and water being the solvent. It was assumed that the overall volume of water and nitrogen would be the same for each sample after degassing. In order for this assumption to be correct, the ratio of nitrogen to water should be the same for each sample. A cutoff value for the percent relative standard deviation (% RSD) of 5% was used. Across all nine samples, a % RSD of 4.2 was observed. Of note, sample NaClO⁻³ appears to be an outlier, thus, when removed, the % RSD drops to 3.4%.

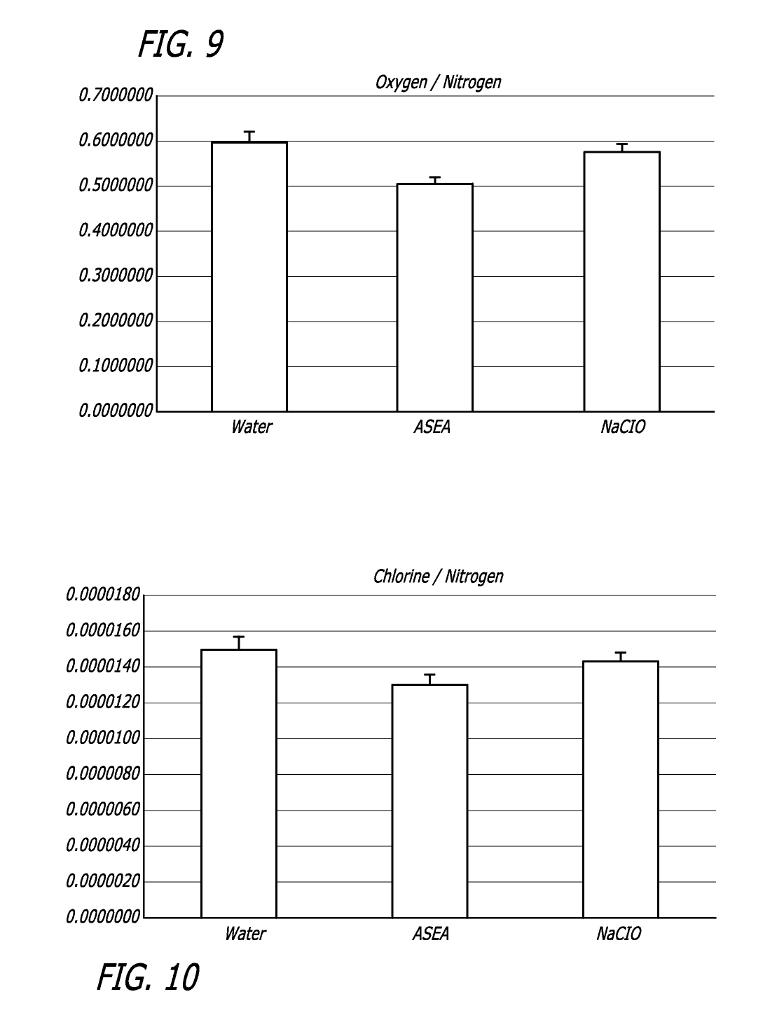

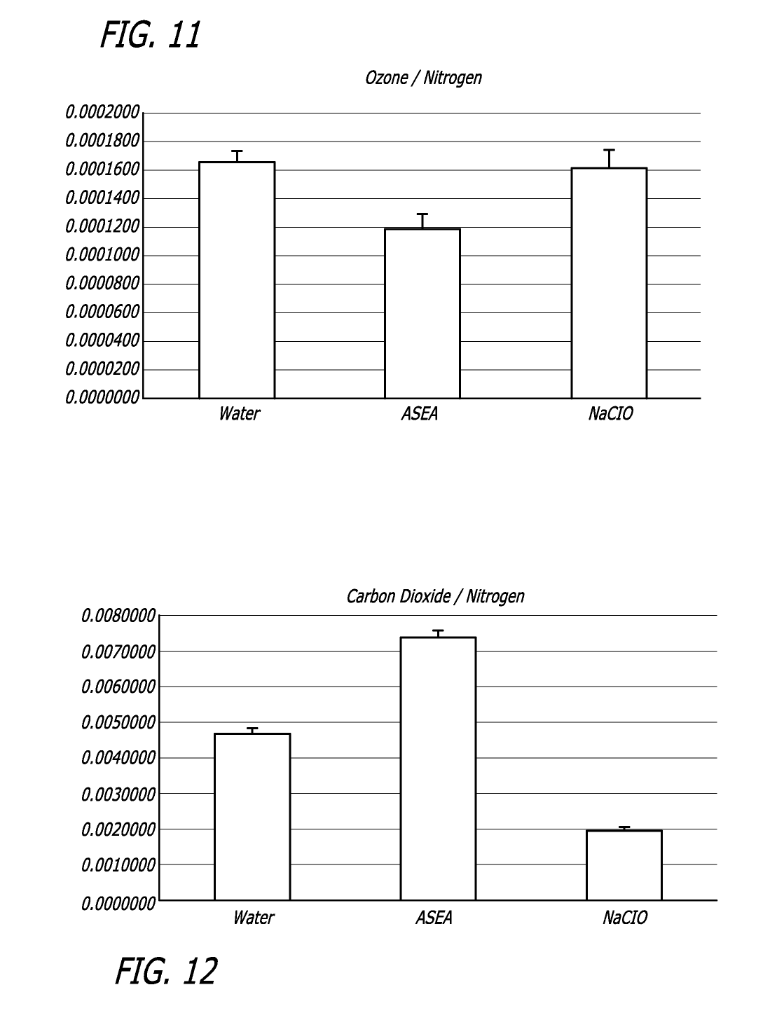

FIGS. 9-11 show the oxygen/nitrogen, chlorine/nitrogen, and ozone/nitrogen ratios. It appears that there were less of these gases released from the beverage than from either water or nitrogen. It should be noted that the signals for both ozone and chlorine were very weak. Thus, there is a possibility that these signals may be due to instrument noise and not from the target analytes.

FIG. 12 shows the carbon dioxide to nitrogen ratio. It appears that there may have been more carbon dioxide released from the beverage than oxygen. However, it is possible that this may be due to background contamination from the atmosphere.

Based on the above, more oxygen was released from both water and sodium hypochlorite than the beverage.

### EPR

Two different beverage samples were prepared for EPR analysis. The beverage with nothing added was one sample. The other sample was prepared by adding 31 mg of DIPPMPO to 20 mL of the beverage (5.9 mM), vortexing, and placing the sample in a 4° C. refrigerator overnight. Both samples were placed in a small capillary tube which was then inserted into a normal 5 mm EPR tube for analysis.

EPR experiments were performed on a Bruker EMX 10/12 EPR spectrometer. EPR experiments were performed at 9.8 GHz with a centerfield position of 3500 Gauss and a sweepwidth of 100 Gauss. A 20 mW energy pulse was used with modulation frequency of 100 kHz and modulation amplitude of 1 G. Experiments used 100 scans. All experiments were performed at room temperature.

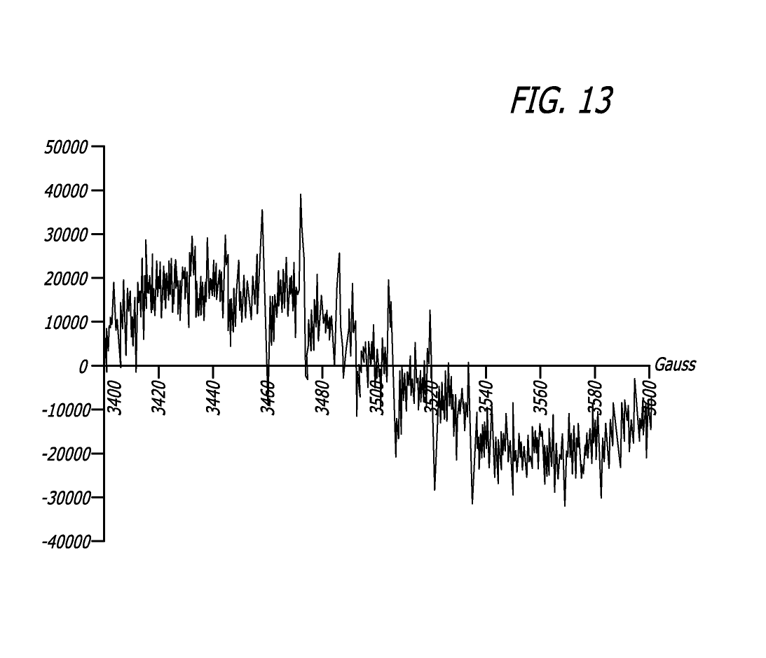

EPR analysis was performed on the beverage with and without DIPPMPO mixed into the solution. FIG. 9 shows the EPR spectrum generated from DIPPMPO mixed with the beverage. The beverage alone showed no EPR signal after 100 scans (not presented). FIG. 13 shows an EPR splitting pattern for a free electron. This electron appears to be split by three different nuclei. The data indicate that this is a characteristic splitting pattern of OH radical interacting with DMPO (similar to DIPPMPO). This pattern can be described by 14N splitting the peak into three equal peaks and 1H three bonds away splitting that pattern into two equal triplets. If these splittings are the same, it leads to a quartet splitting where the two middle peaks are twice as large as the outer peaks. This pattern may be seen in FIG. 13 twice, with the larger peaks at 3457 and 3471 for one quartet and 3504 and 3518 for the other quartet. In this case, the N14 splitting and the 1H splitting are both roughly 14G, similar to an OH radical attaching to DMPO. The two quartet patterns in FIG. 13 are created by an additional splitting of 47 G. This splitting is most likely from coupling to 31P, and similar patterns have been seen previously. The EPR spectrum in FIG. 13 indicates that there is a DIPPMPO/OH. radical species in the solution.

### Example 3

### Delivery of Beverage to Exercising Mice

Studies have shown that supplementation with green tea extract for 8-10 weeks in mice resulted in increased treadmill time to exhaustion compared to control mice. Higher muscle glycogen and increased fatty acid beta-oxidation were measured in exercised mice treated with green tea extract. Based on these studies, further exploration into other supplements that can increase physical properties such as time to exhaustion, VO₂ₘₐₓ, and the like may be useful.

The effect of ASEA ingestion on treadmill endurance capacity, fuel substrate utilization, tissue inflammation, and tissue oxidative stress in mice was studied. If ASEA causes increased fatty acid mobilization then endurance capacity can be improved in mice taking ASEA (compared to placebo). Sparing of muscle glycogen can be seen when taking ASEA. Mice were given the equivalent of about half the human ASEA dose.

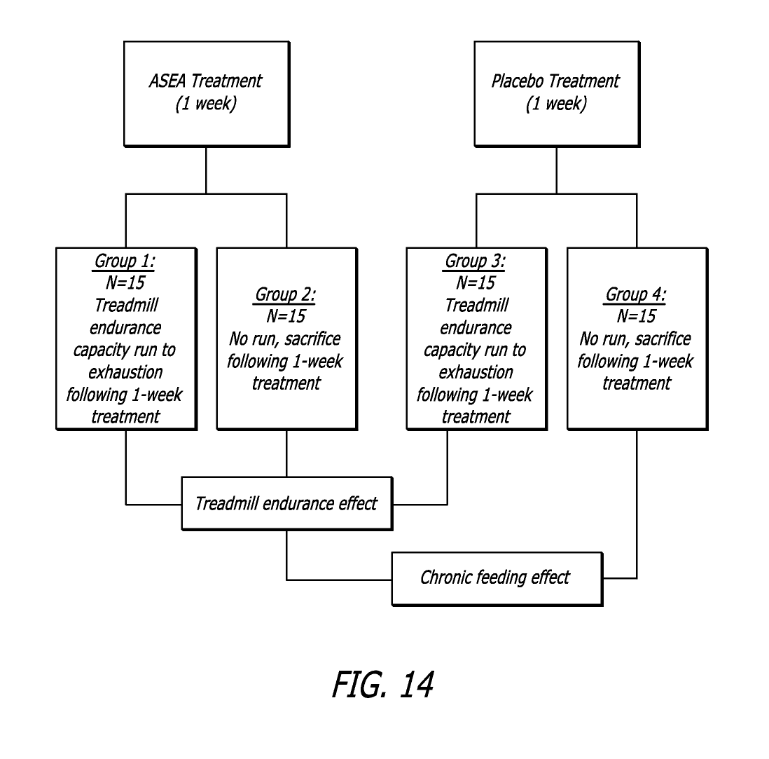

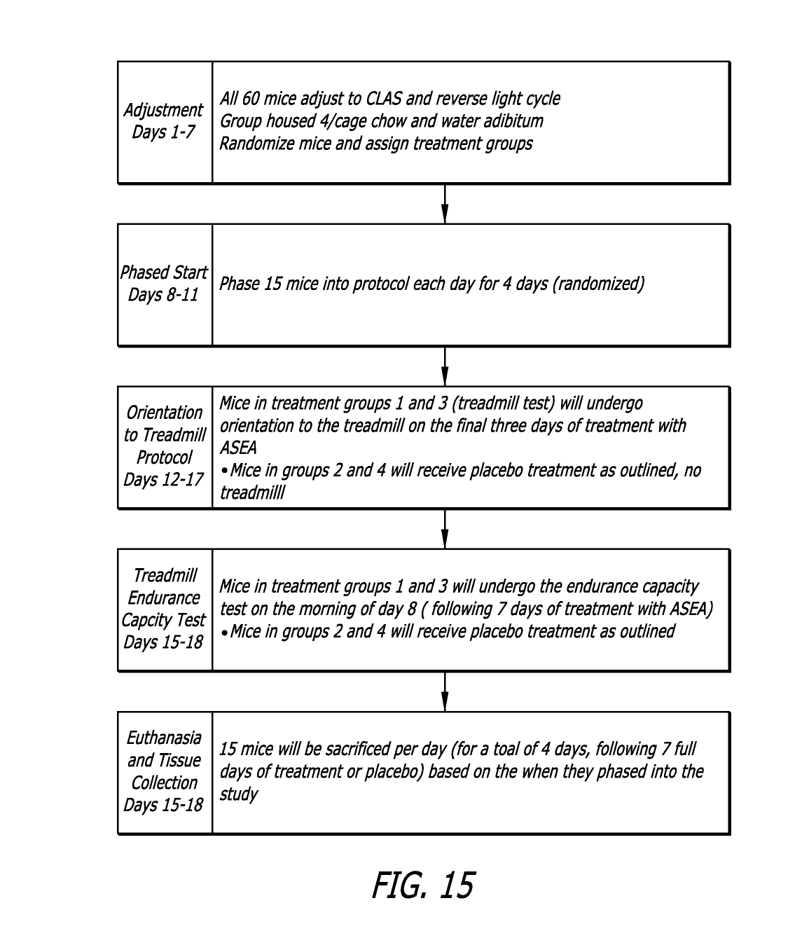

Six-month old male specific pathogen-free C57BL/6 laboratory mice (n=60) were purchased from Jackson Laboratory. Mice were randomly assigned to each of four treatment groups (n=15 each) as illustrated in FIG. 14. A total overview of the mouse preparation and study is illustrated in FIG. 15.

This particular strain and model of mouse has been used in previous studies involving both exercise and nutritional intervention studies. Thus, the use of this strain allowed comparison to data from other studies. Mice can be a suitable substitute for humans for this type of study because mice are genetically similar to humans and thus data obtained in this study will be translatable to human intervention studies.

All animal procedures took place in the Center for Laboratory Animal Sciences (CLAS) at the North Carolina Research Campus and protocols were reviewed and approved by the Institutional Animal Care and Use Committee (IACUC).

ASEA or placebo (same ingredients as ASEA beverage without the proprietary signaling molecules added) was administered to the mice via gavage once per day for 1-week. The average body mass of all the mice at the start of the study and the volume of ASEA used for the gavaging were determined, but the volume did not exceed 0.3 mL per mouse. Guidelines for gavage are as the follows: “the volume should not exceed 1-2% of body weight (=0.2-0.4 ml for a 20 g Mouse)”. Thus, a volume of 0.3 mL for a 6 month old 30 g mouse is well below this volume suggestion.

The beverage was not palatable and the mice did not drink it willingly. Gavage was an acceptable alternative to ensure the mice did not become dehydrated simply because they would not drink the study beverage. The gavaging was performed by the animal husbandry staff at CLAS.

Following the 1-week (7 days) treatment period mice were euthanized and tissues harvested for further analysis of outcome measures. The four groups of mice were phased into the 1-week protocol each day. For example, if Group 1 started the protocol on a given day, Group 2 would begin the protocol on the following day, Group 3 would be begin the following day, and Group 4 the day after that. Mice from Group 1 would then be euthanized following the final treadmill test (7th day of treatment), Group 2, Group 3, and Group 4 each on subsequent days. Thus, total time for the mouse protocol was 11 days. There was overlap of orientation treadmill days, with maximal treadmill testing and euthanasia days. As stated, prior to euthanasia, mice from Group 1 and Group 3 underwent an endurance treadmill test to exhaustion using the protocol summarized in the following Table.

| Time | Speed | |
|---|---|---|
| (min) | (m/min) | Notes |
| 1 | 0 | Adjustment to treadmill |
| 5 | 10 | “warm up” period |
| 2 | 12 | |
| 2 | 14 | |
| 2 | 16 | |
| 2 | 18 | |
| 2 | 20 | |
| 2 | 22 | Speeds between 20-24 m/min correspond to |
| | | roughly 80% VO₂ₘₐₓ for mice |
| 2 to end | 24 | Mice stay at this speed until they reach exhaustion |
| | | (e.g., sit on shock grid for 5 full seconds) |

During the three day period preceding the maximal endurance test, mice were oriented (trained) to the treadmill for 15 min/day. Speeds for the training days were about 10 m/min, 15 m/min, and 18 m/min respectively. Then, on the final day of treatment mice underwent a maximal endurance capacity test on the treadmill (Table 1).

Mice from Group 2 and Group 4 were not submitted to an endurance capacity test and were euthanized at the end of 1-week treatment. Tissues harvested from these mice were collected to assess the chronic effects of the test beverage in absence of an exercise intervention. All blood/plasma and tissues were snap-frozen in liquid nitrogen and stored at −80° C. until assayed.

For the treadmill orientation and endurance protocols, mice were run on a multi-lane rodent treadmill (Columbus Instruments, Columbus Ohio) equipped with a shock grid at the back. Once each mouse was placed in a treadmill lane, a 1 minute resting period was initiated. At this point, the mouse was able to adjust to the inside of the treadmill chamber. Following the 1 minute rest period, the treadmill belt was started at a speed of about 10 m/min, and the protocol described in the above Table was followed.

Mice were allowed to run until they were no longer able to keep up with the belt and the hind limbs stayed on the shock grid for more than about 5 seconds. When the mouse was no longer running (as assessed by sitting on the shock grid with all 4 paws off of the belt for more than 5 seconds), the mouse was removed from the shock grid immediately and placed back into the home cage. The mice were then monitored for recovery for a period of at least about 20 minutes following the orientation bouts.

The maximal endurance test occurred only once per mouse, and mice were euthanized immediately following the test. The test ended when the mouse could not run off the shock grid onto the treadmill at any point during the test or if signs of exhaustion (signs of above normal heart rate and ventilation) were evident.

The signs of exhaustion used included a mouse sitting on the shock grid for more than 5 seconds, rapid breathing, and/or increased heart rate. It has been our experience that mice that are not fatigued do not show these signs and will continue to run within 5 seconds of stopping. These procedures follow national recommendations (American Physiological Society's, Resource Book for the Design of Animal Exercise Protocols, 2006) based on research in the area. If at any point during the test a mouse got its foot caught between the shock grid and the treadmill the test was immediately terminated. If the mouse was injured and needed treatment, proper procedures were followed and vivarium staff was notified. If the mouse was deemed not injured, it was allowed to recover and placed back in its home cage and re-tested the following day. Once the mouse completed the protocol the mouse was placed back into its home cage. Generally, mice are usually back up and jumping around the cage within 30 seconds of re-exposure to the home cage following an endurance test. However, mice were still monitored several times during the 20-60 minutes following the procedure and notes were taken of any abnormalities such as apathy or decreased food consumption.

Some form of motivation was needed to make the mice run on the treadmill, particularly in the orientation sessions. A variety of forms of motivation can be used. The three most common techniques are, use of shock grid, use of air puffs, and manually tapping a mouse's tail. Use of air puffs have the potential to be ineffective and possibly confounding to data analysis. Given the standard rodent treadmill that is used in this type of testing that encloses the treadmill, manually tapping the tail was not ideal. Thus, shock grids were the best method of motivation for exercise on the treadmill.

The shock grid was positioned at the back of the treadmill. The shock grid delivered pulsed shock at an average current of 1.0 milliamperes at 150 volts (the shock grid was adjustable within a range of 0-3.4 mA). The shock grid was regularly checked with an ampmeter to ensure proper functioning. The shock levels used were 22 times less than that accepted in the literature. Also, the amperage of the system was 167-500 times less than lethal levels for mice, and the total power of the system was 60 times less than lethal levels for mice. No new data or guidelines existed to suggest that the use of a shock grid with our proposed settings was anything but appropriate.

Based on a similar study the effect size was calculated to be 1.647. Using p=0.0125 for significance during a priori power analysis. Using G-Power the following calculation was made and a least significant number of animals is assumed to 12/group. We propose 15 animals per group (with estimated power of 0.95) to account for any loss of power if any animals do not make it through the protocol.

### Analysis: A Priori: Compute Required Sample Size

| Input | Tail(s) | Two |
|---|---|---|
| | Effect size d | 1.6470588 |
| | α err prob | 0.0125 |
| | Power (1-β err prob) | 0.95 |
| | Allocation ratio N2/N1 | 1 |
| Output | Noncentrality parameter δ | 4.5106563 |
| | Critical t | 2.6694793 |
| | Df | 28 |
| | Sample size | 15 |
| | Actual power | 0.9604227 |

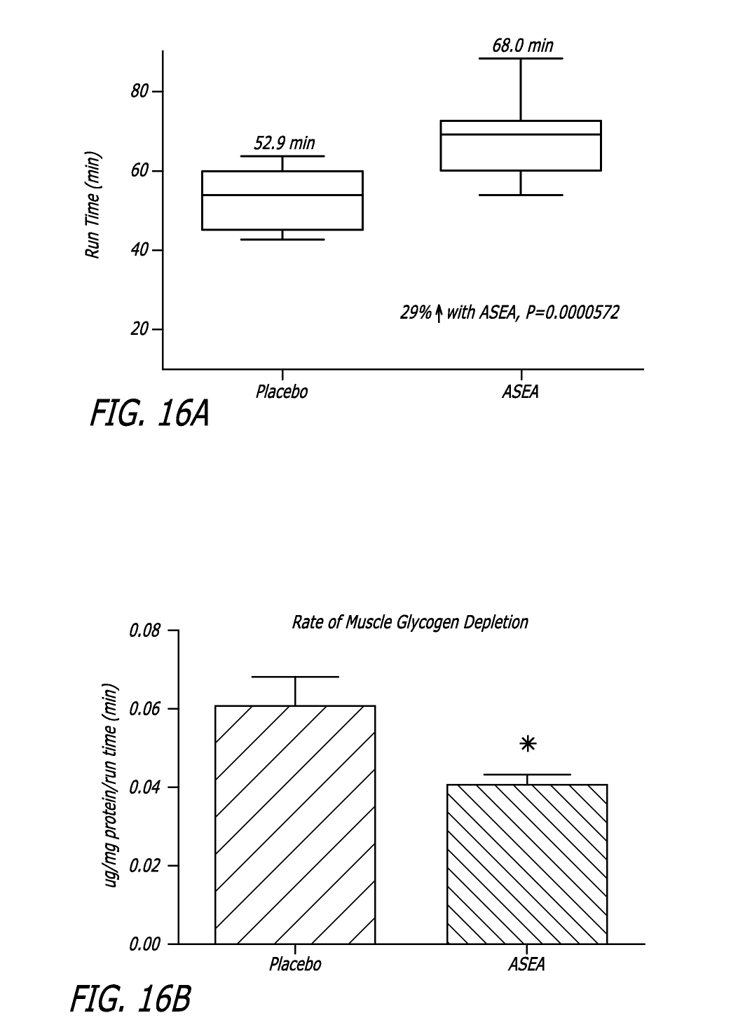

Based on the results from the mouse study, results are illustrated in FIGS. 16-20. FIG. 16 illustrates that mice who were administered ASEA had an increased run time to exhaustion. As such, ASEA can be used to increase time to exhaustion in athletes when exercising.

Sparing of muscle glycogen can be seen when taking ASEA (FIG. 16B). Mice that were administered ASEA had on average about a 33% reduction in rate of muscle glycogen depletion. These results suggest that mice taking ASEA used less glycogen/minute of exercise. Muscle glycogen sparing may explain the increase in endurance performance and the increase in V0₂ₘₐₓ.

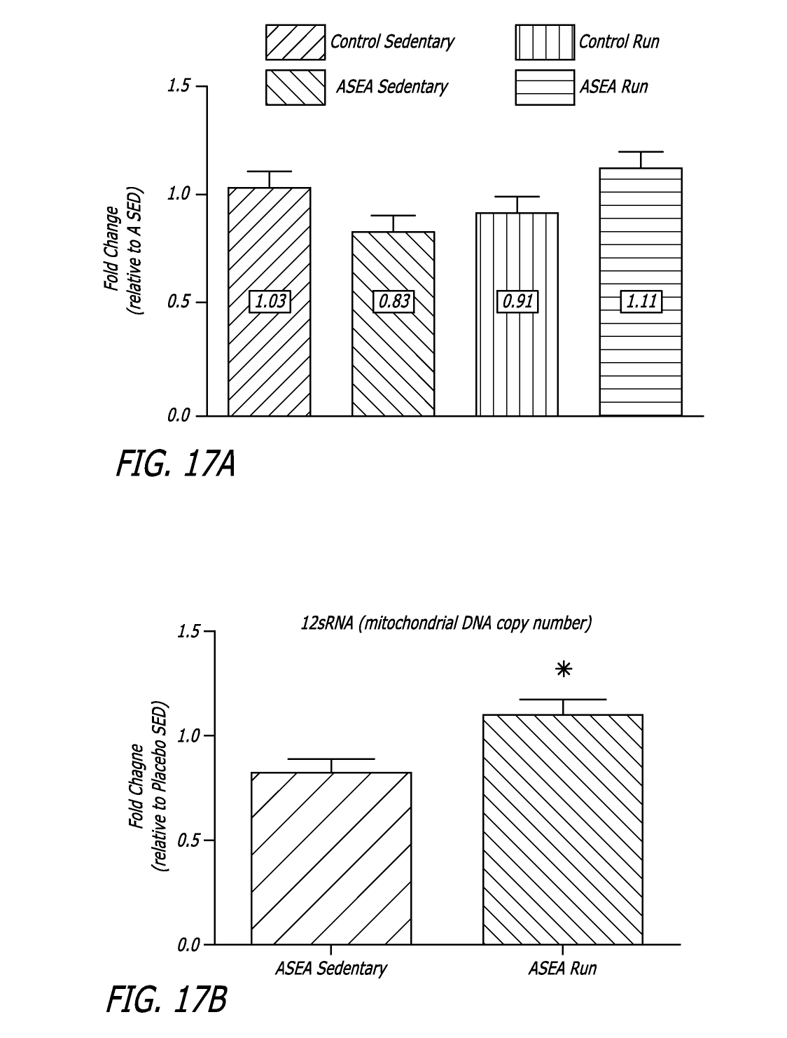

FIG. 17 illustrates the fold change relate to ASEA of different mouse groups; P=0.042. This measurement tracks 12sRNA (mitochondrial DNA copy number). One week ASEA consumption in sedentary mice did not increase muscle mitochondria density. An interaction between one long endurance exercise bout to exhaustion was observed with ASEA vs. ASEA sedentary (P<0.05). Fold change increased when ASEA was delivered along with exercise, but fell when exercise was not present. This supports that ASEA helped decrease the level of oxidative stress in the muscle.

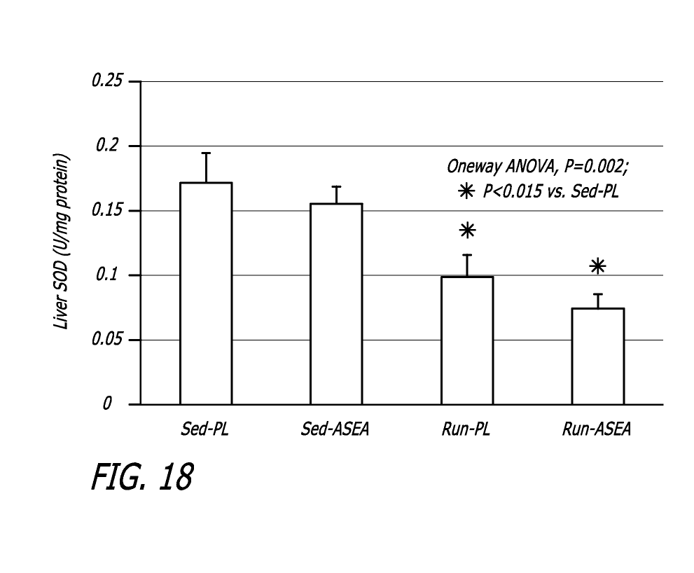

FIG. 18 illustrates that SOD produced in the liver decreases in mice when administered ASEA and subjected to exercise. U is the amount of enzyme needed to inhibit 50% dismutation of the superoxide radical. An acute bout of exercise activates CuZnSOD activity, but most studies reported no change in its mRNA and enzyme protein levels, suggesting that the increased activity was due to increased OZ concentration. This result can indicate that ASEA linked to exercise can reduce oxidative stress.

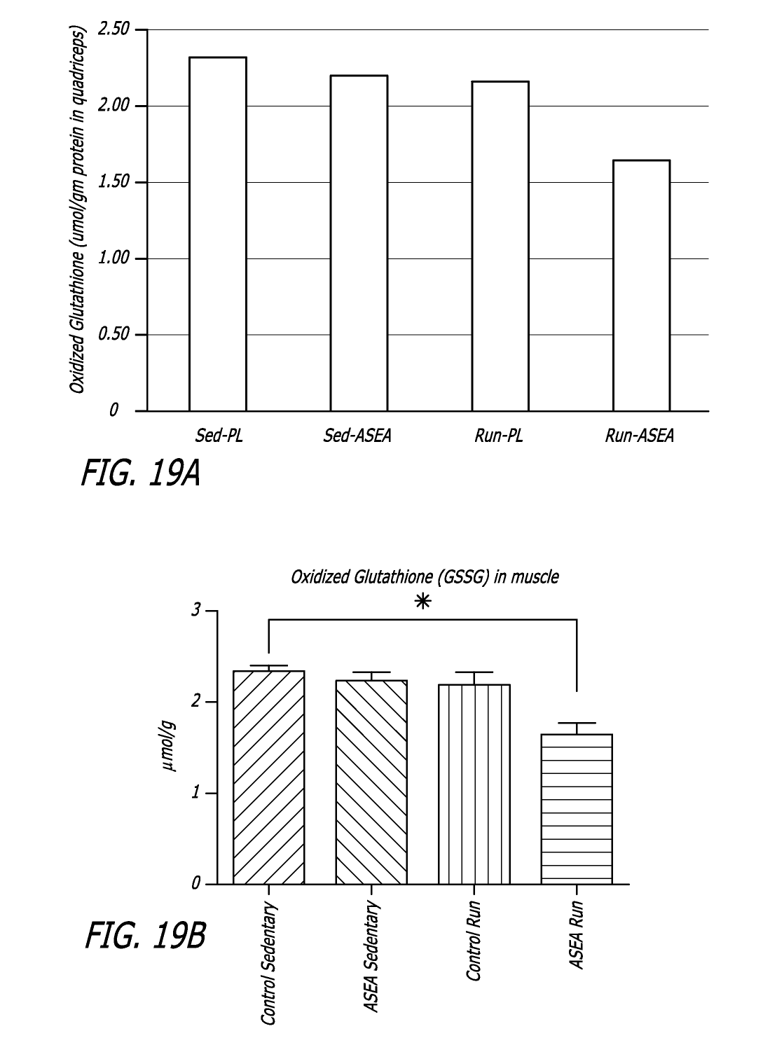

FIGS. 19A and 19B illustrate that oxidized glutothione decreases in mice when administered ASEA and subjected to exercise. This result can indicate that ASEA linked to exercise can reduce oxidative stress.

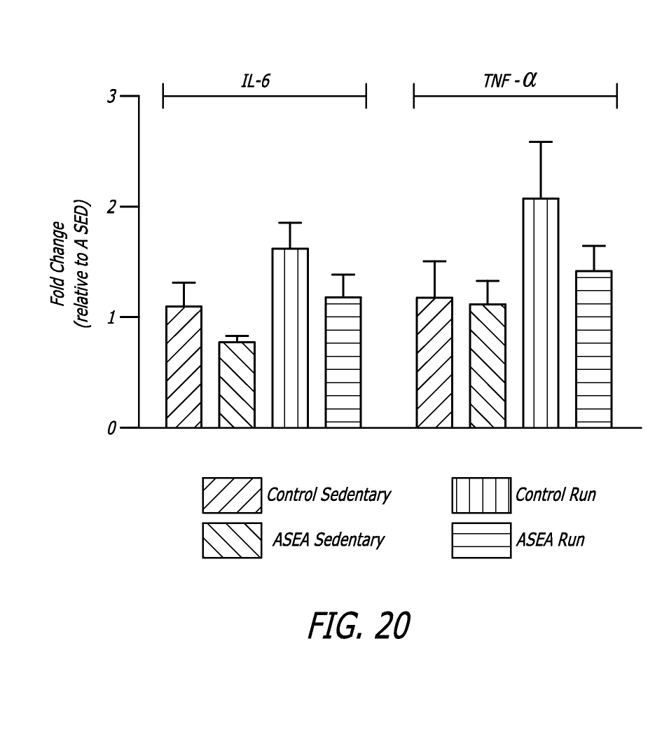

FIG. 20 illustrates that exercise increased mRNA (gene expression) for IL-6 and TNF-alpha, indicating the typical pro-inflammatory response. ASEA tended to reduce gene expression for these inflammatory cytokines.

### Example 4

### Human Biking Exercise Study

A study was performed to estimate the increase in metabolism of individuals using the present systems and methods wherein the subjects drank an ASEA beverage(s). The study was performed at the Metabolomics Laboratory, North Carolina Research Campus, David H. Murdock Research Institute and Appalachian State University. A goal of the study was to measure the influence of ASEA on small molecules (metabolites) that can shift in response to supplementation. The shift in metabolites, depending on the nutritional product, may represent effects on inflammation, oxidative stress, and physiologic stress.

Twenty-two subjects participated in the study. Each subject was tested for baseline values of VO₂ₘₐₓ and body composition. Then, ten participants were given an ASEA beverage once a day for seven days and ten subjects were given a placebo once a day for seven days. See FIG. 1.

On the day of the first phase of the study, blood and urine were collected from all twenty-two participants and then each of the twenty-two participants biked 75 km. Blood and urine were collected just prior to finishing the 75 km biking and one hour thereafter. Results are tabulated below.

A washout period of three weeks then lapsed throughout which participants did not entertain an ASEA beverage. After the three weeks, the participants crossed-over and were given the opposite beverage for 7 days. The same routine was again performed (blood urine, 75 km biking, blood urine, blood urine one hour post). Data is tabulated below.

Athletes on ASEA for seven days started the 75 km cycling trial with high blood free fatty acids leading to increased fat oxidation and a sparing of amino acids (and potentially muscle glycogen).

Serum creatinine and urea can also increase post-exercise and did in the study. The liver and/or kidney may be a contributor to this post-exercise increase in serum creatine and urea levels.

Chronic effects of ingesting ASEA prior to exercise can be higher fatty acid levels pre-exercise (several types of fatty acids). An acute effect can be increased fatty acid oxidation and mobilization during exercise (placebo condition was linked to a late mobilization). Also, triglyceride mobilization can correspond with the increase in free fatty acids as glycerol was higher at baseline which can be indicative of extensive adipose triglyceride hydrolysis.

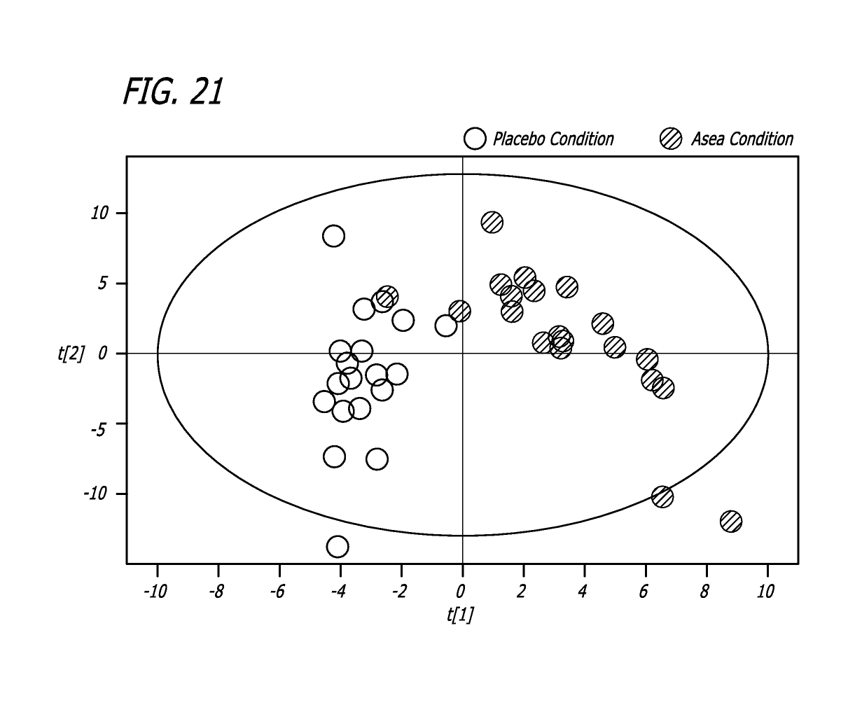

Based on the results the PLS-DA model visualized a distinct separation in global metabolic scores between treatment conditions [R²Y (cum)=0.814, Q2Y (cum)=0.712]. Blue: placebo condition; Red: ASEA condition. See FIG. 21. As illustrated in Fuigure 21, ingestion of 4 fl oz/d ASEA for one week caused an extensive shift in 43 metabolites (especially free fatty acids, fructose, amino acids, Kreb cycle intermediates), shifting the entire metabolome in these 20 cyclists.

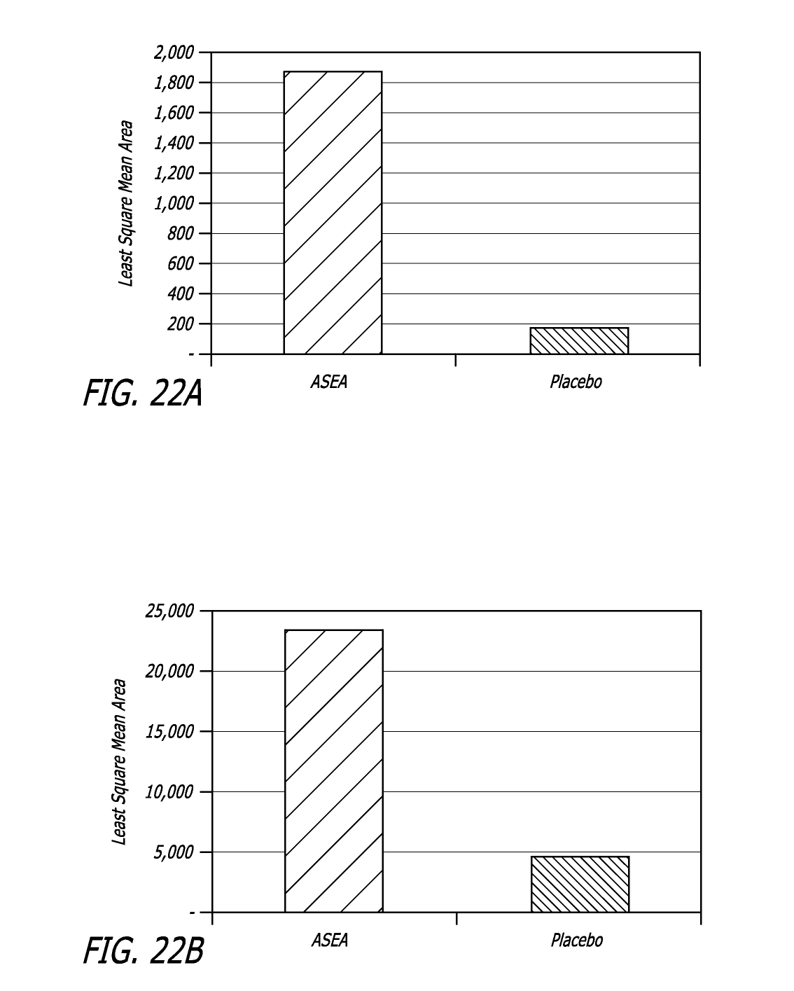

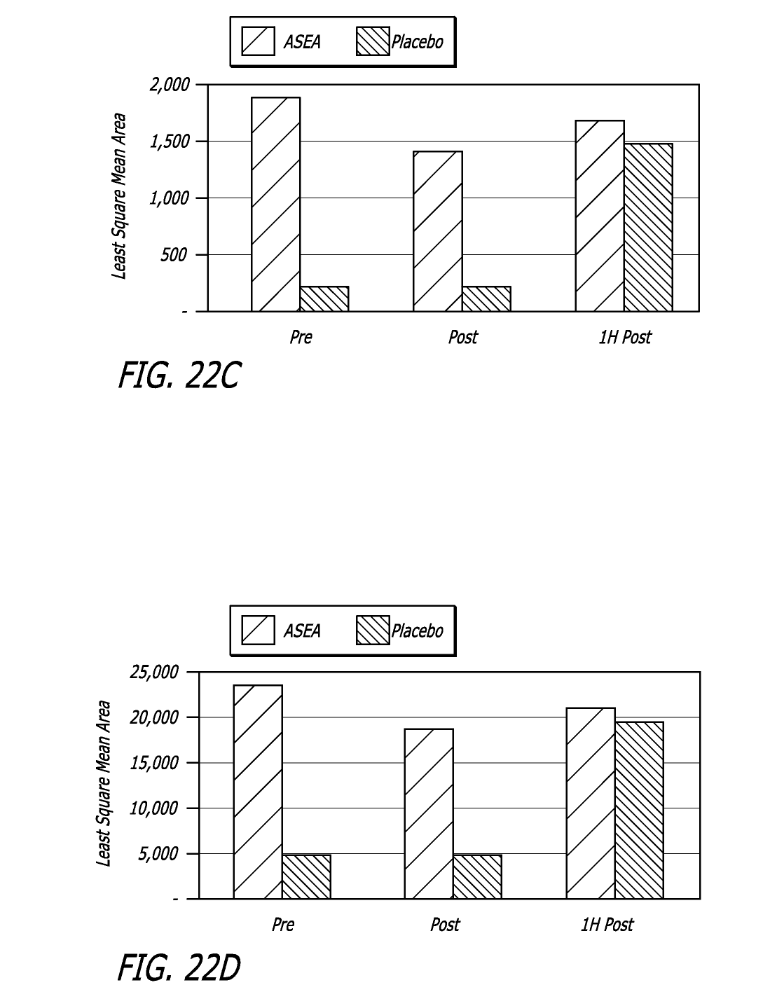

Ingestion of ASEA beverage for one week strongly increased serum fatty acids levels during exercise. These fatty acids were likely from adipose tissue. Increases can be seen in FIGS. 22A-D.

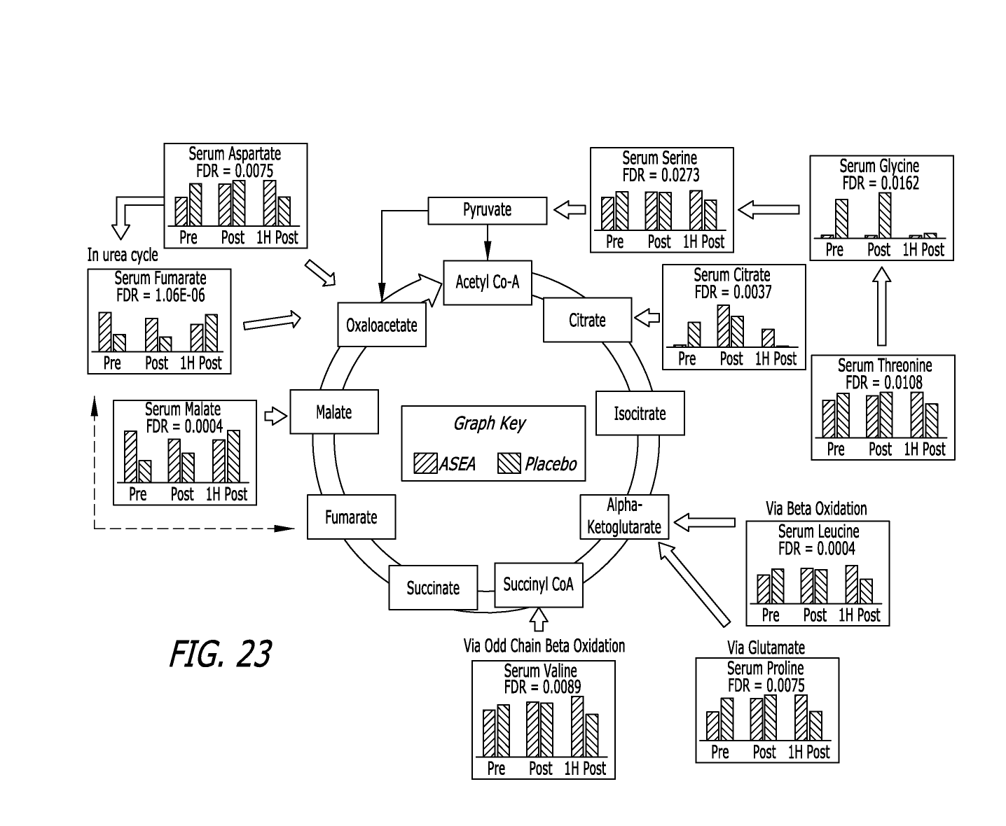

Further, as illustrated in FIG. 23, high levels of blood free fatty acids were linked to a sparing of amino acid catabolism, and increased Krebs Cycle intermediates, post-exercise. Intermediates and products of interest include aspartate, serine, glycine, citrate, threonine, leucine, proline, valine, malate, and fumarate.

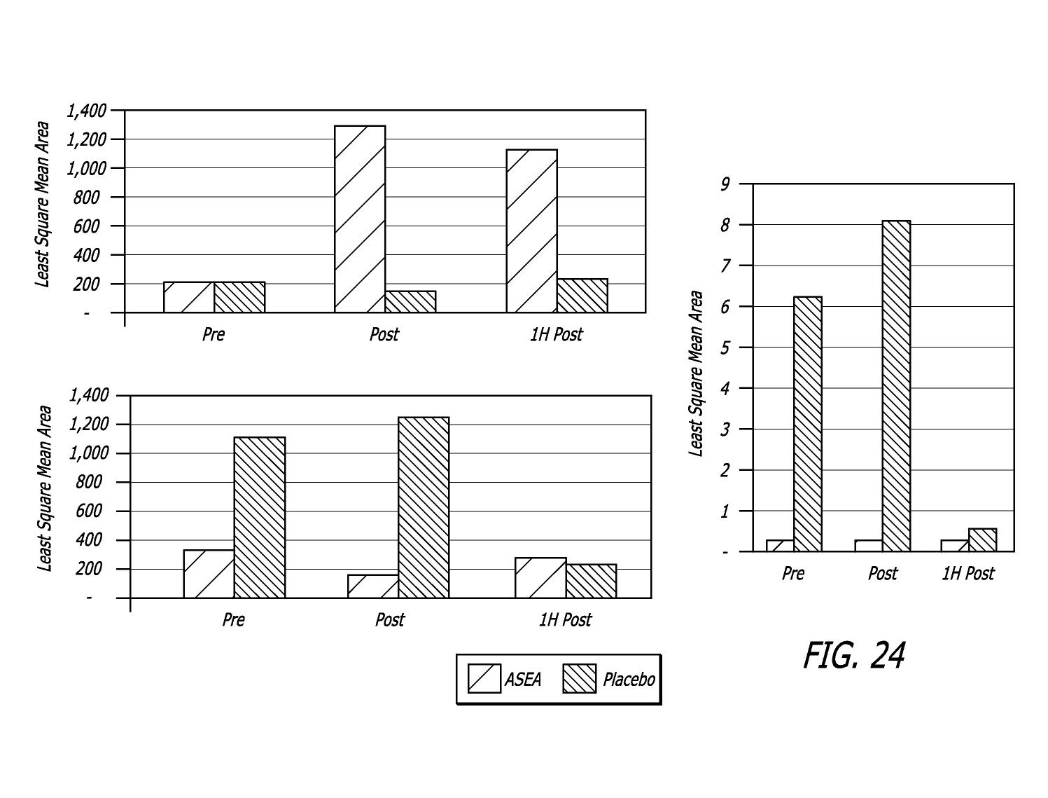

As illustrated in FIG. 24, ASEA supplementation affects ascorbic acid both acutely and chronically. Ascorbic acid appears to increase post exercise and 1 hour post exercise. Fructose and threonic acid appear to be lower as compared to placebo.

### Example 5

### Metabolic Profiling Study

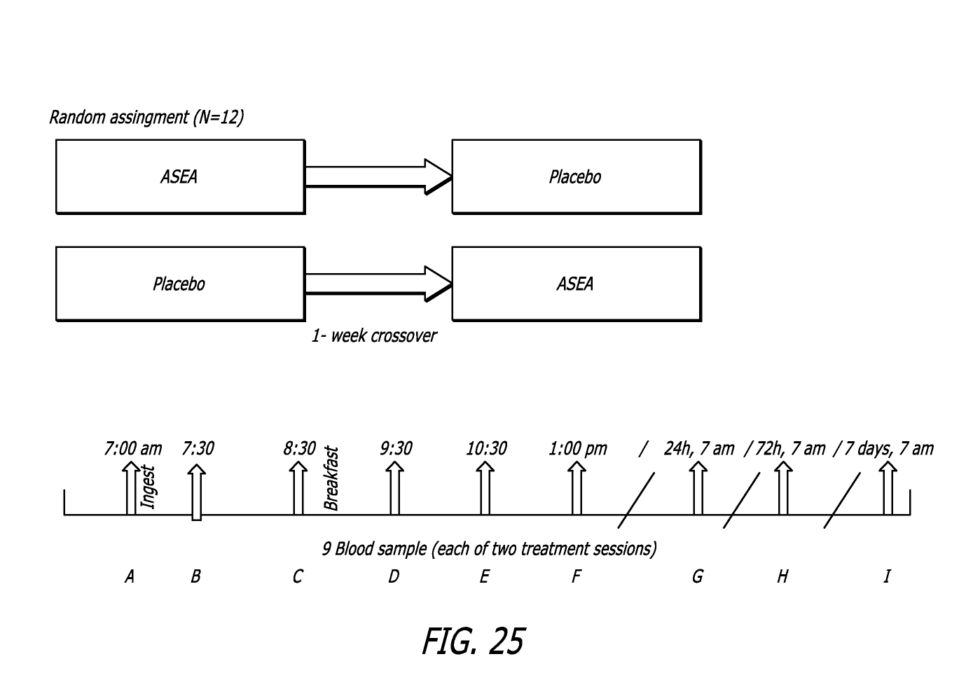

Twelve individuals were profiled using GC/MS technique to probe metabolic markers. Individuals were randomly assigned ASEA beverage or placebo to perform the study and then a one week cross-over period, followed by performance of the opposite condition. The routine followed for the study is illustrated in FIG. 25.

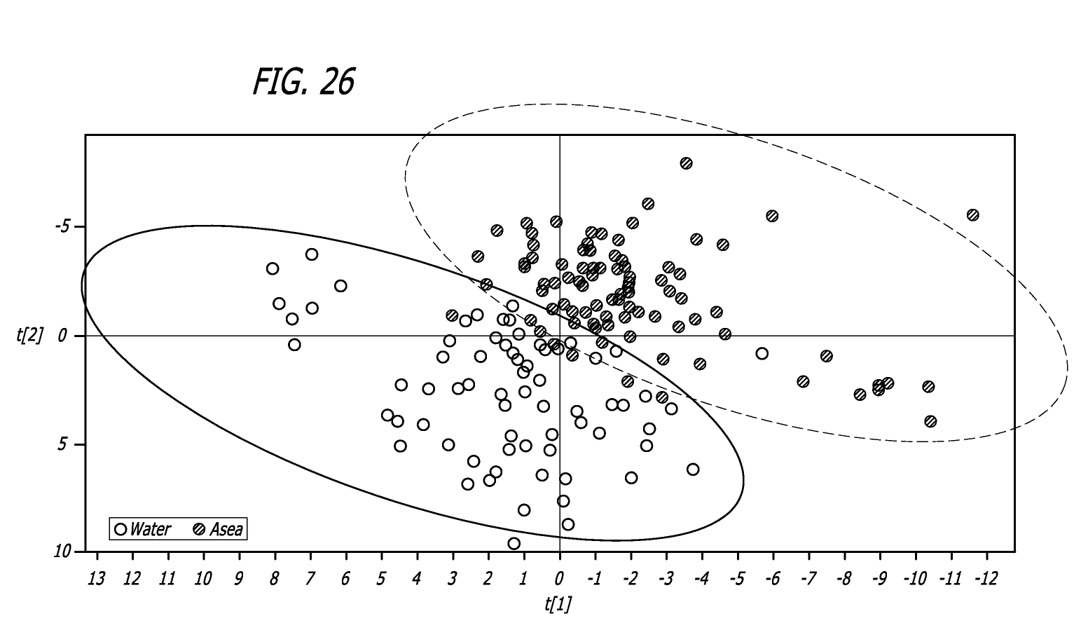

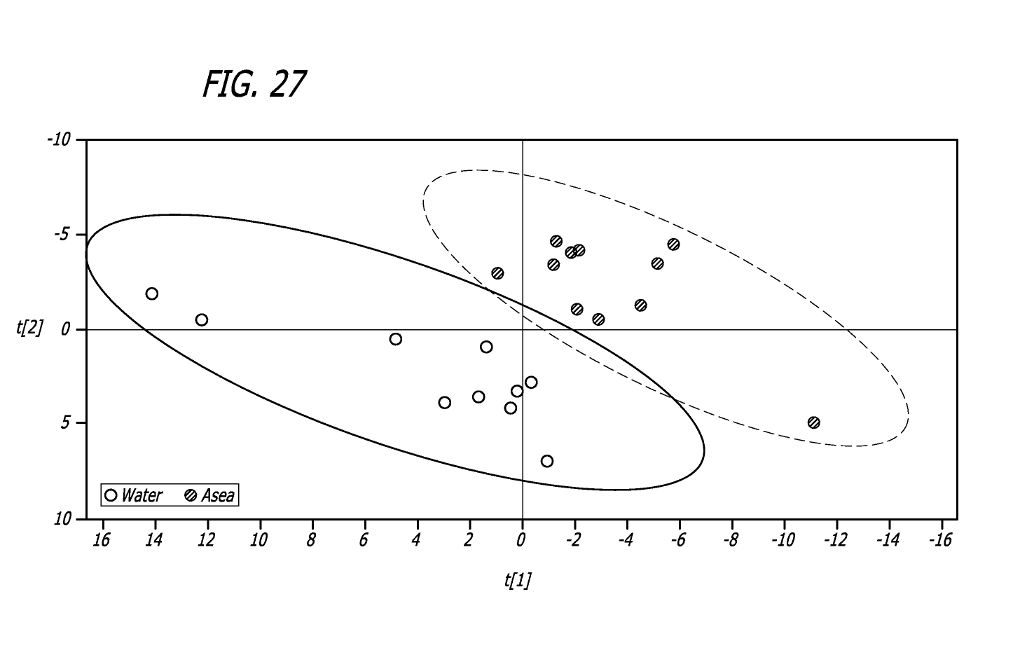

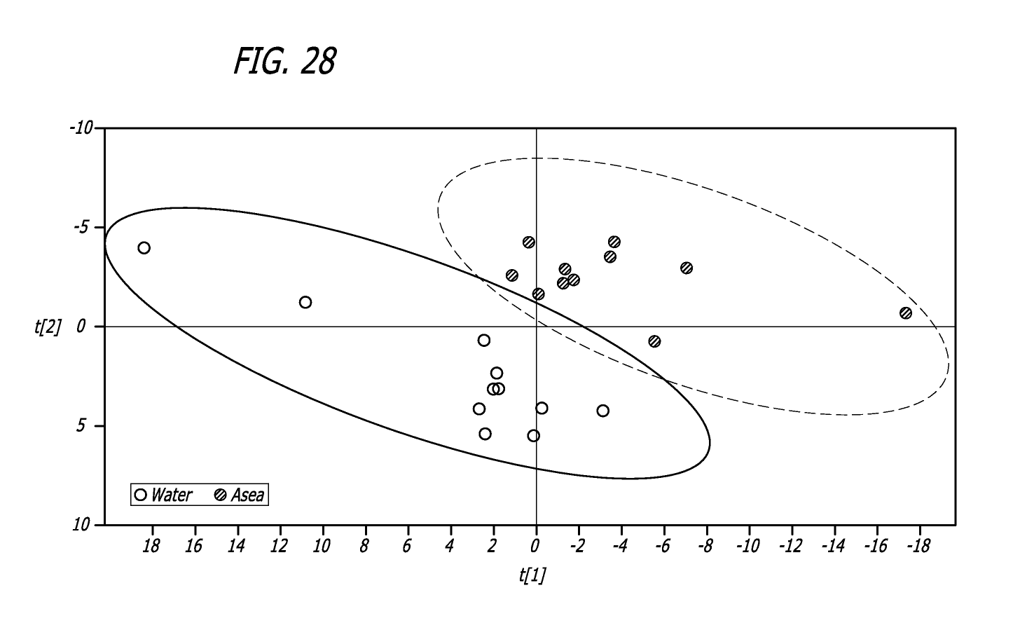

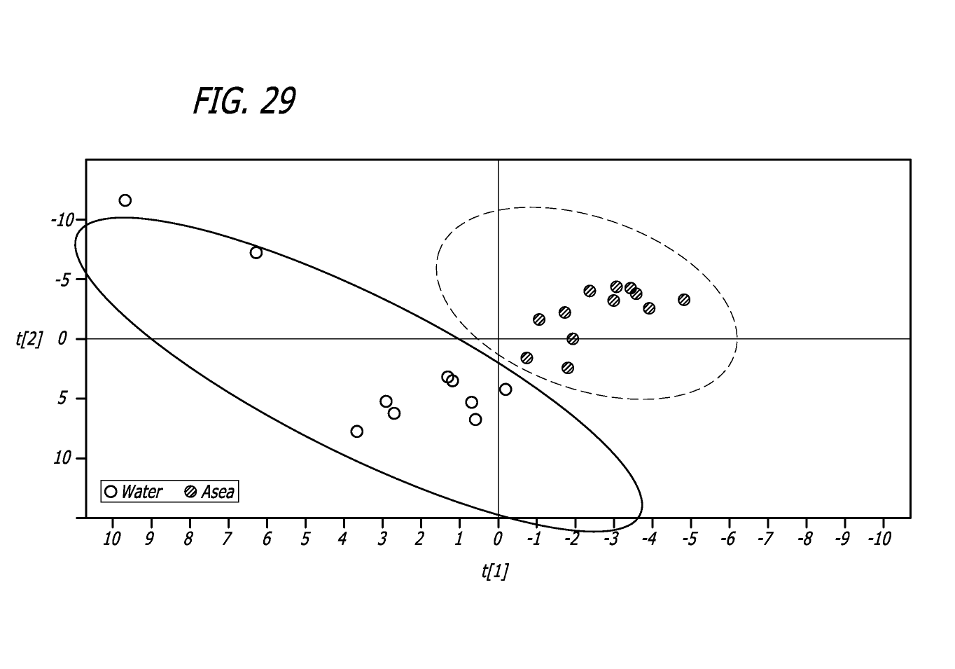

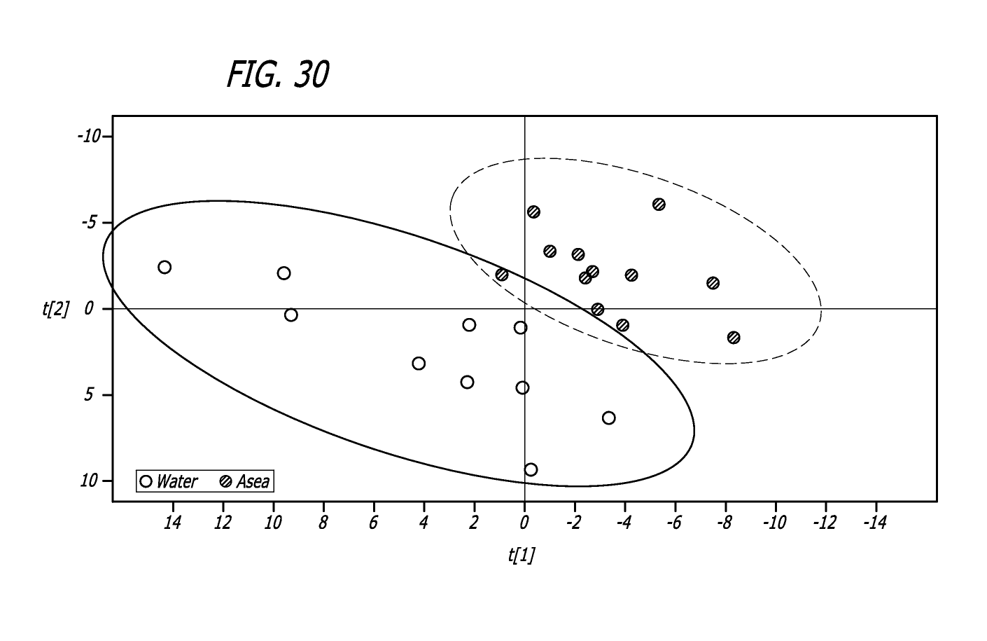

Based on the 9 samples collected from each individual on each portion of the study, data processing detected 98 known and 117 unknown metabolites. FIGS. 26-30 illustrate PLS-DA compared A-B ratios between conditions. FIG. 27 illustrates 30 minutes post ingestion, FIG. 28 illustrates 1.5 hours post ingestion, FIG. 29 illustrates 3.5 hours post ingestion, and FIG. 30 illustrates 24 hours post ingestion.

At 30 minutes post ingestion of ASEA, shifts in the following metabolites were seen: d-fructose, d-xylose, glycerol 2-phosphate, 2-oxo-4-methylvaleric acid, sorbose, and octadecanoic acid. At 90 minutes post ingestion of ASEA, shifts in the following metabolites were seen: proline, mannose, L-valine, allo-isoleucine, glycine, and citrulline. At 150 minutes post ingestion of ASEA, shifts in the following metabolites were seen: fumaric acid, 3-amino-2-methyl-propanoic acid, L-aspartic acid, ethanolamine, 1,2-propanediol-1-phosphate, and aminomalonic acid. At 3.5 hours post ingestion of ASEA, shifts in the following metabolites were seen: threitol, nonanoic acid, salicylic acid, L-glutamine, nona-decanoic acid, and hexadecanoic acid. At 6 hours post ingestion of ASEA, shifts in the following metabolites were seen: aminomalonic acid, succinic acid, threitol, pyruvic acid, alpha-hydroxyiso-butyric acid, and L-cysteine. At 24 hours post ingestion of ASEA, shifts in the following metabolites were seen: glycine, L-methionine, alanine, L-lysine, ribitol, and L-tyrosine.

### Example 6

### Human Running Performance

A study to determine if ASEA versus placebo ingestion during a 2-week period improves run time to exhaustion when athletes run on treadmills with the speed adjusted to 70% VO2max.

Blood and skeletal muscle biopsy samples are collected and analyzed for shifts in metabolites and glycogen utilization, respectively, to study underlying mechanisms.

Metabolites and glycogen utilization are altered when ASEA is used alongside exercise.

### Example 7

### Efficacy of Ingesting ASEA on Disease Risk Factor Change in Overweight/Obese Women

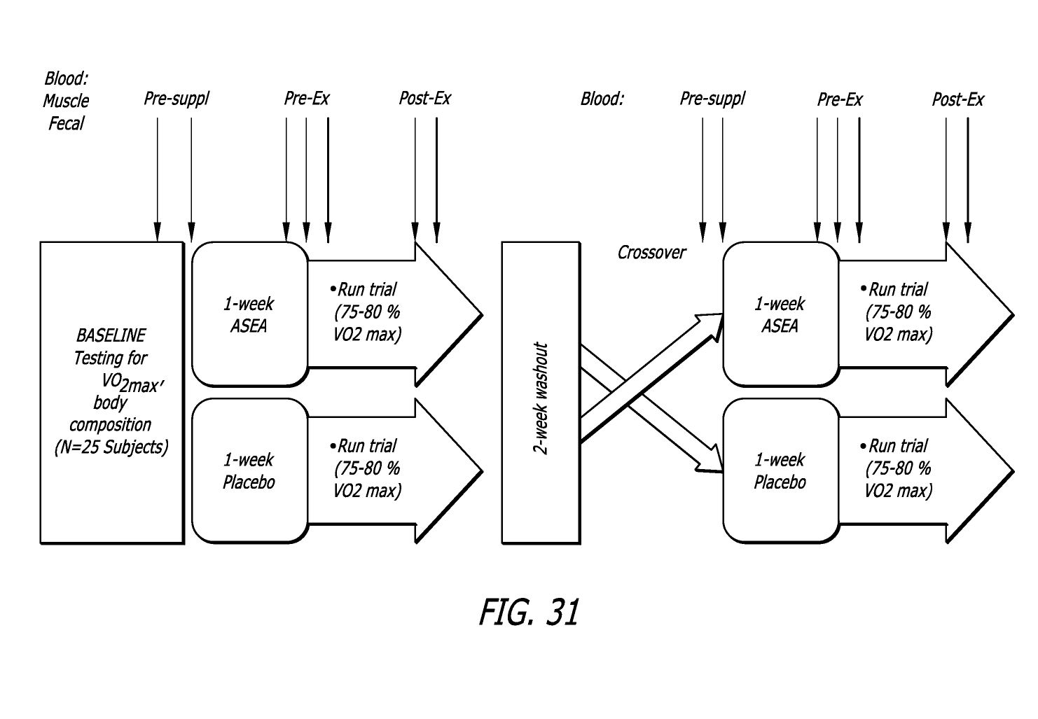

A 12-Week, randomized trial is performed accord to the protocol in FIG. 31. The study evaluates the effectiveness of 4 fl. oz./day ASEA compared to placebo over a 12-week period in helping adult women improve disease risk factors associated with arterial stiffness, inflammation, cholesterol status, blood pressure, oxidative stress and capacity, fasting serum glucose, and metabolic hormones.

After ingestion of ASEA over a 12 week period decreases arterial stiffness, decreases inflammation, improves cholesterol status, decreases blood pressure, decreases oxidative stress and capacity, decreases fasting serum glucose, and alters metabolic hormones.

### Example 8

### Effect of an Immune-Supporting Supplement, ASEA, on Athletic Performance

Described is a pilot study used to measure the possible effects of an immune-supporting supplement on athletic performance as measured by a standard VO2max and Ventilatory Threshold (VT) athletic endurance test.

The objectives of the pilot study are to (1) confirm the general observation that an immune-supporting supplement has an effect on athletic performance and (2) determine the specific physiological parameters: Heart Rates (HR), volume of O2 inspired (VO2), volume of CO2 expired (VCO2), volume of expired gas (VE), Respiration Rate (RR), Respiratory Exchange Ratio (RER), Aerobic Threshold (AeT), Anaerobic Threshold (AT), VO2max and Ventilatory Threshold (VT) that are affected by oral ingestion of this supplement during both the aerobic and anaerobic phases of exercise.

The immune-supporting supplement, ASEA™, contains a balanced mixture of Redox Signaling molecules that purportedly increases the efficiency of the communication channels between cells, enabling faster response of the immune system and cellular healing activities. Enzymes in the body also break down these Redox Signaling molecules into salt water and nascent oxygen.

There are two proposed mechanisms involving Redox Signaling that can affect athletic performance, (1) increased efficiencies in cellular absorption or use of oxygen, prolonging aerobic metabolism, and (2) more efficient processing of lactate energy stores and tissue repair mechanisms, prolonging anaerobic metabolism.

During physical activity, the increased power requirements from muscle tissues require increased metabolism of available energy stores. Sustainable aerobic metabolism of sugars can supply this energy demand as long as there is an adequate supply of oxygen and sugars in the blood. As energy demands exceed the ability of the respiratory and cardiovascular system to deliver sufficient oxygen to the muscle tissue, methods involving the anaerobic metabolism of carbohydrates, creatines, pyruvates, etc. start to become prevalent.

Anaerobic metabolism supplies the excessive demand for energy but is accompanied by the production of CO2 and lactates. Prolonged or excessive anaerobic metabolism depletes the available energy stores faster than they can be renewed; the buildup of CO2 and lactates can also interfere with aerobic metabolism and thus, when the energy stores are spent, exhaustion will result.

Since anaerobic metabolism is marked by an excess in CO2 and lactate production, it can be monitored by measuring the excess CO2 exhaled during exercise or the buildup of lactates in the blood. The Ventilatory Threshold (VT) is the point where the excess CO2 is first detected in the expired breath; it is related to the point at which anaerobic metabolism is starting to become prevalent.

In this pilot study, VT was determined graphically from the VCO2 vs. VO2 graph. VCO2 is the volume of CO2 expired per minute and VO2 is the volume of 02 inspired per minute. VO2max is simply the maximum volume of 02 inspired per minute possible for any given individual. VO2max is measured in ml/kg/min (milliliters of O2 per kilogram of body weight per minute). VO2max is measured at the peak of the VO2 curve. The Aerobic Threshold (AeT) was determined by the software and indicates when fat-burning metabolic activities start to be dominated by aerobic metabolism. The Anaerobic Threshold (AT) was also software-determined and marks the point where the anaerobic metabolism starts to completely dominate.

Recruitment Methods: A standard VO2max test was run on 18 athletes who responded to recruitment flyers posted in athletic clubs and to invitations extended to a local competitive Triathlon team. The participants were selected based on answers from qualification questionnaire which affirmed that they:

1. Perform a rigorous physical workout at least five hours per week on average.

2. Have no medical conditions that might prevent participation

3. Agree to follow diet and hydration instructions.

4. Will perform only normal daily routines during the study.

5. Have no history of heart problems in the family.

The final selections were athletes of a caliber much higher that the expectations reflected in the recruitment flyers, a majority being athletes involved in regular athletic competitions. All of the participants had never taken the supplement prior to the study.

The participants did not receive any monetary compensation, but did receive a case of product and results from the VO2max tests.

The VO₂ₘₐₓ testing was done at an athletic club by accredited professionals holding degrees in exercise physiology and with more than 10 years daily experience in administering VO₂ tests. The participants were given a choice of performing the test on either a treadmill or a stationary cycle. A CardioCoach® metabolic cart measured heart rate (HR), inspired and expired gases (VO₂, VCO₂, VE) and recorded weight, height, age, and body mass indexes (BMI). Power settings on the treadmill or cycle were recorded every minute.

Each participant was scheduled to take two VO₂ₘₐₓ tests, (1) a baseline test and (2) a final test. The baseline test was performed before any supplement ingestion. The participants drank 4 oz. of the supplement per day between the baseline test and the final test (7 to 10 days later) and drank 8 oz. of the supplement ten minutes before starting the final test. For the baseline test, the power settings on the cycle or treadmill were determined by the test administrator. The power settings for the final test were matched exactly to the power settings of the baseline test for each participant. Participants were encouraged to strictly maintain their regular diet and exercise routine and to come to each test well hydrated (at least 8 oz. of water in the last 2 hours before each test).

Each participant was fitted with a breathing mask and heart monitor. Each VO2max test consisted of a 10 min. warm up period where participants walked or cycled at a low power setting determined by the administrator. This was followed by a ramp up period, where the administrators increased the power settings every minute, according to their evaluation of the physical condition of the participant, and termination when the administrators started seeing the indications of a maximum VO2 reading when RER (VCO₂/VO₂)>1.0 or at the administrator's discretion. The administrators had ample experience in obtaining consistent VO2max results on this equipment, estimated at about 6% test to test variation over the last 5 years.

The raw data (HR, VO₂, VCO₂, VE, Power Settings) were collected from the CardioCoach® software for analysis. Data points were automatically averaged over 15 to 25 second breath intervals by the software, VO2max is also determined by the software with an averaged VO2 peak method. VT was determined graphically from the slope of the VCO2 vs. VO2 graph.

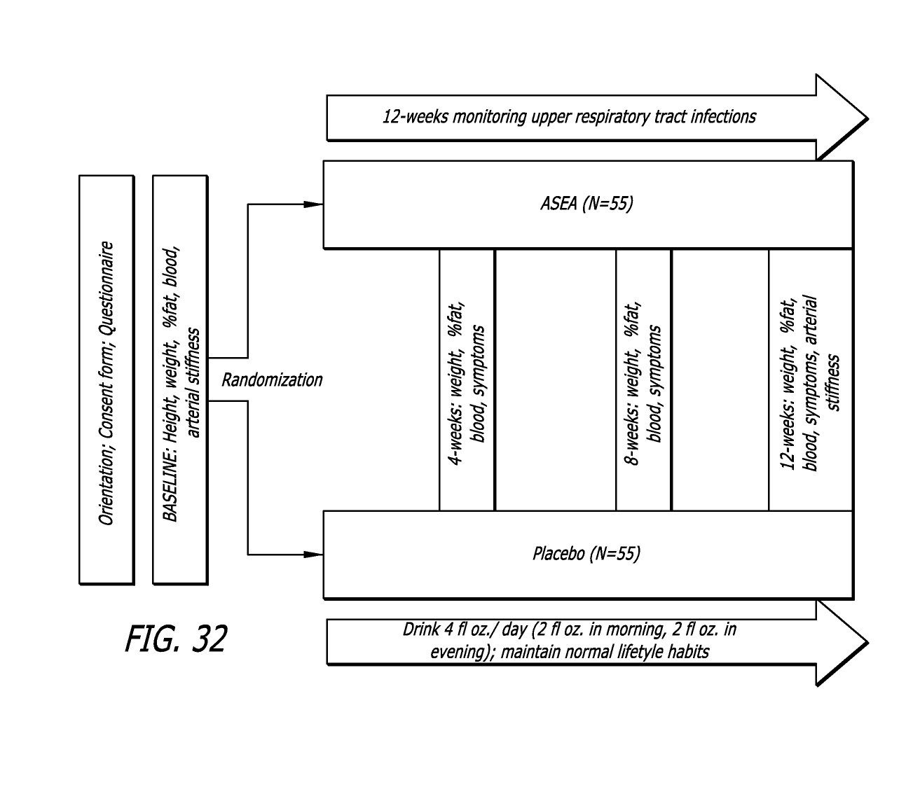

Linear regression methods were used to determine the slope, change in VCO2 over change in VO2. In theory, when aerobic metabolism switches to anaerobic metabolism, the volume of CO2 expelled (VCO2) is increased in proportion to the Volume of O2 inhaled (VO2). This is reflected as an increase of slope on the VCO2 vs. VO2 graph, seen as a clear kink on the graph around the VT point. Linear regression was used to determine the slope both before VT and after. Slopes were determined by linear regression on the linear region of data points before and after VT point, excluding points surrounding the VT and near VO2max. The intersection of the before and after lines was used to determine the reported VT point (FIG. 32).

Methods for determining the VT point on any individual participant were kept consistent from the baseline test to the final test. Average HR was averaged over the linear range of HR increase during the power ramp, excluding points a few minutes into the beginning and before the end of the data set. In every case, the same data analysis methods were used for the final test as were used for the baseline test for each participant.

Compliance to protocol was very high by both participants and administrators, based on answers to compliance questions. One data set was discarded for low VCO2 values, probably due to a loose mask. The ventilatory data for this one participant was rejected, leaving 17 valid data ventilatory data sets. The Heart Rates (HR), however, were compared for all 18 participants.

| Total | | | Average | | | |
|---|---|---|---|---|---|---|
| Partici- | Average | Male/ | Weight | Cycle/ | Average | Data Sets |
| pants | Age | Female | (Kg) | Treadmill | BMI | Selected |
| 18 | 41 ± 9 | 16/2 | 76 ± 11 | 7/11 | 24.4 ± 3.4 | 17 |

The average VO₂ₘₐₓ reading over all participants (N=17) was measured at the relatively high value of 62.5 ml/kg/min, indicative of the quality of athletes in the sample. Only four participants had VO₂ₘₐₓ readings below 55 ml/kg/min; these four were not involved in competitive training programs.

The data shows that two significant changes in physiological parameters could be attributed to ingestion of the supplement, as determined by a statistical paired t-test analysis. The average time taken to arrive at VO2max was increased by 10% with very high confidence (P=0.006) and the average time taken to arrive at Ventilatory Threshold (VT) was increased by 12% with a marginal level of confidence (P=0.08).

Given that the power ramp-up-points between the baseline and final test for each participant were identical, an increase in the amount of time to obtain VO2max and VT on the final test also indicates a higher average power outputs at such thresholds. Calibrated power output measurements were not available, however, the test administrator for the final test, upon reaching the maximum power recorded for the baseline test, regularly surpassed this maximum power before the participant reached VO2max on the final test.

All other physiological parameters (VO₂ₘₐₓ, VT, AeT, AT, Start HR, HR at AeT, HR at AT, HR at VO₂ₘₐₓ, and overall average HR) were not significantly changed by supplement ingestion. The high level of consistency between the baseline and final test for these parameters, however, supports the repeatability of the tests. The test to test repeatability has an estimated standard deviation of less than 5% for all parameters.

| Averages | | | | | |
|---|---|---|---|---|---|
| (N = 17) | Baseline | Final | Change | % Change | P-Value |
| VO₂ₘₐₓ | 62.5 | 63.6 | +1.1 | +2% | — |
| (ml/kg/min) | | | | | |
| VT | 36.4 | 38.7 | +2.3 | +6% | 0.34 |
| (ml/kg/min) | | | | | |
| Aerobic | 43.6 | 43.8 | +0.2 | +0% | — |
| Thresh. (AeT) | | | | | |
| Anaerobic | 55.5 | 56.5 | +1.0 | +2% | — |
| Thresh. (AT) | | | | | |
| Pre VT | 1.030 | 1.030 | 0.0 | 0% | — |
| Slope of | | | | | |
| VCO₂/VO₂ | | | | | |
| Post VT | 1.997 | 1.944 | −0.053 | −2.7% | — |
| slope of | | | | | |
| VCO₂/VO₂ | | | | | |
| Start Heart | 87.4 | 85.9 | −1.5 | −1% | — |
| Rate (bpm) | | | | | |
| Heart Rate | 147 | 145 | −2 | −2% | — |
| at AeT | | | | | — |
| Heart Rate | 165 | 165 | 0 | 0% | — |
| at AT | | | | | |
| Heart Rate | 174 | 175 | +1 | +1% | — |
| at VO₂ₘₐₓ | | | | | |
| Heart Rate | 137 | 134 | −3 | −2% | — |
| Overall | | | | | |
| Time to VT | 306 | 344 | 38 | +12% | 0.08 |
| (secs) | | | | | |
| Time to VO₂ₘₐₓ | 639 | 703 | 64 | +10% | 0.006 |
| (s) | | | | | |

Of the 17 participants in the study, 70% of them experienced a significant increase in time to VO2max, 18% of the participants showing more than a 25% increase, 41% showing more than a 10% increase, 18% of the participants exhibiting no significant change and 12% showing a mild decrease (under 10%).

There was a moderate but significant correlation between the increases in “time to VO₂ₘₐₓ” and “time to VT” (correlation coefficient 0.35), meaning that an increase in time to reach VO2max was moderately but not always proportional to the increase in the time it took to get to VT. There is a strong correlation between increase in time to VO2max and decrease in the average overall heart rate (correlation coefficient−0.67), meaning that an increase in time to VO2max would most often be accompanied by a decrease in average overall heart rate.

Ingestion of the test supplement, ASEA™, for 7-10 days prior to and immediately before a VO₂ₘₐₓ test, was shown to significantly increase the time it took for 70% of the participants to reach VO2max under equivalent carefully regulated power ramp-up conditions. Time to VT likewise was significantly extended.

The extension of time to reach VT, under similar increasing demands for energy, is a direct indication that the aerobic phase of metabolism is being extended and/or the anaerobic phases somehow are being delayed as the demand for energy increases.

The lack of any other changes in the physiological parameters (VO₂ₘₐₓ, VT, AeT, AT and associated heart rates) suggests that cardiovascular capacity, lung capacity and blood oxygen capacity and regulation are not affected. This assumption is reasonable, given that the short duration of this study excluded the possibility of training effects.

One feasible explanation for the results lies in the enhancement of aerobic efficiencies, meaning that more aerobic energy can be extracted at the same physiological state, or that the clearance of lactates or CO₂ becomes more efficient, again allowing greater aerobic efficiency. Note that “time to AeT” and “time to AT” were not compiled in this study, however changes in these parameters would be expected and might offer clues to determine the underlying mechanisms.

The results of this pilot test indicate that there is a strong case for athletic performance enhancement and further investigation is warranted. A placebo-based double-blind test, measuring the more subtle effects in ventilation and heart rates along with increases in blood lactate levels during a controlled, calibrated power ramp would provide defensible evidence for this effect and better support for some specific underlying mechanisms of action.

### Example 9

### In Vitro Bioactivity Study

Described are a variety of preliminary results from in vitro experiments, performed at national research institutions, investigating the bioactivity of a certain redox signaling compound, ASEA™, when placed in direct physical contact with living cells. Specific investigations include in vitro toxicity and antioxidant efficiencies of the master antioxidants glutathione peroxidase (GPx) and Superoxide Dismutase (SOD) inside living cells and the translocation of two well-studied transcription factors (NF-kB, NRF2) known to regulate toxic response and antioxidant production in human cells. Some preliminary work on concentration dependence was also done as well as cell proliferation, counts associated with induced oxidative stress in human cells.

The objectives of the investigations were (1) to determine if any signs of toxicity (NF-kB activation) are manifest when varying concentrations of a certain redox signaling compound, ASEA™, are placed in physical contact with living cells, (2) to determine if such direct contact affects the antioxidant efficacy of glutathione peroxidase (GPx) and superoxide dismutase (SOD) and (3) to determine if such contact activates translocational transcription (NRF2) associated with increased expression of antioxidants in living human endothelial cells and to verify the expression of such transcription factors by Western Blot analysis, (4) to determine the effect of this redox signaling compound on proliferation cell counts of human cells and associated markers (LDH) for cell viability and health, (5) to determine the effects of this redox signaling compound on cells that were stressed with cytokines (Cachexin), radiation and serum starvation.

The immune-supporting Redox Signaling supplement, ASEA™, contains a redox-balanced mixture of Redox Signaling molecules [both reactive oxygen species (ROS) and reduced species (RS)] that are involved in a large variety of pathways and receptor-site activity in human cells. For example, when cells are damaged, for any reason (ex. toxins, DNA breaks or infections), the native Redox Signaling messengers inside the cells can become imbalanced, most often manifest by the accumulation of intracellular oxidants and ROS (oxidative stress). The cell, so affected, will activate defense and repair mechanisms aimed to restore proper redox-signaling homeostasis and proper cellular function. If repair efforts are unsuccessful and normal homeostatic redox balance is not able to be restored, then within a few hours, the excess oxidants and ROS in such cells will facilitate apoptotic processes to internally digest and destroy the dysfunctional cell. Healthy neighboring cells will then divide to replace it. A complete field of science called “redox signaling” has been founded to study such processes, with literally thousands of references available.

It is the nature of certain redox signaling molecules, when unbalanced or isolated, to elicit immediate recognizable toxic responses in exposed living cells; hydrogen peroxide is one example of such a redox signaling molecule. The first-line cellular response to toxic substances involves the translocation of NF-kB into the nucleus as a precursor to the inflammatory response and other defense mechanisms. The movement of NF-kB into the nucleus can be visibly tracked in a living cell under a fluorescence microscope with the aid of fluorescent tag molecules. The observation of nuclear translocation of NF-kB is a sure marker that a toxic response has been initiated. Even low-level toxicity is detectable with this catch-all method; low-level concentrations of hydrogen peroxide, for example, produce an easily distinguishable positive toxic response.

A separate transcription factor, NRF2, moves into the nucleus in response to low-level oxidative stress and facilitates the increased production of antioxidants. Again, by the use of fluorescent tags, the nuclear translocation of NRF2 can be seen in cells under a fluorescence microscope. NRF2 nuclear translocation is a second-line-of-defense mechanism known to increase the production of protective enzymes and antioxidants such as glutathione peroxidase and superoxide dismutase. NRF2 translocation will often accompany low-level NF-kB activation and NF-kB activation (almost) always precedes NRF2 translocation. Substances that exhibit low-level toxicity, such as trace homeopathic toxins, have long been used to activate the NRF2 pathway in order to stimulate these natural defend-repair-replace mechanisms.

Enzymatic efficacy of antioxidants, such as Glutathione Peroxidase (GPx) and Superoxide Dismutase (SOD), can be determined through standardized ELISA tests that measure the time-related reduction of certain oxidants introduced into cell lysates after the living cells have been exposed to the test substance for a given period of time. The reagents of the ELISA test must be chosen as not to interfere or interact with the test substance. Other critical factors such as the time of exposure and concentration dependence must be experimentally determined.

Western Blot methods also exist to experimentally determine the quantities of GPx or SOD in cell lysates. These well-established molecular separation techniques and can be used to directly verify whether the quantity of such antioxidant enzymes has been increased in the sample. Measured antioxidant efficiency, however, remains the best indication of cellular antioxidant defense.

Monitoring cellular proliferation, cell counts and chemical indicators of cellular death are also commonly used to determine cellular viability and gross response to stressors such as radiation, cytokines and toxins. Cachexin, for example, is a potent toxin, a cytokine, that elicits immediate toxic responses and build-up of oxidative stress in exposed cells. Cells, so stressed, exhibit a greater tendency to undergo apoptosis and die, thereby releasing internal proteins (such as LDH) into the surrounding serum.

Normally, when the introduction of such stressors and toxins elicit oxidative stress conditions in the cell cultures, cell counts will fall, cellular proliferation will subside, and serum LDH levels will rise, indicating that cell death is occurring in the culture. Hydrogen peroxide, radiation and serum starvation can also elicit similar responses. Redox signaling messengers, as outlined above, are intimately involved in cellular reception of and response to such stressors; redox messengers are involved in mediating antioxidant production and action to protect the cells, repair mechanisms necessary to fix DNA and structural damage and also in mediating the apoptotic process that results in cell death.

Increasing the concentration of such redox messengers in the serum may serve to augment the efficiency of these normal cellular processes. The exact action of various redox signaling mixtures must be determined experimentally. Independent unpublished studies, involving Mass Spectroscopy, Florescent Spectroscopy and Electron Spin Resonance, have unmistakably verified the existence of several kinds redox signaling molecules in the immune-supporting supplement, ASEA™. Well-established redox electrochemistry also validates the existence of such redox signaling molecules. The stability of this redox-balanced mixture is many orders of magnitude greater than expected. The confirmed preservation of unstable moieties in this supplement might be explained by the existence of certain stable molecular complexes, some of them verified by mass spectroscopy, that can shield radical interactions. Intellectual property agreements, however, prevent the disclosure of the details.

The following research was conducted on a best efforts basis by a senior researcher at a national laboratory and is designed to assess basic mode-of-action when the redox signaling, ASEA™, is placed into direct contact with human cells:

1. The initial dose range projected for in vitro studies was extrapolated from a 10 ml ASEA/kg equivalent oral dose from human trials.

2. Glutathione peroxidase (GPx) and superoxide dismutase (SOD) ELISAs were used to determine whether ASEA alters enzymatic activity in murine epidermal (JB6) cells.

3. LDH (non-specific cellular death) levels and cell proliferation rates were determined for various cell types exposed to ASEA.

4. Human microvascular endothelial lung cells (HMVEC-L) were treated with ASEA and cell lysates were analyzed by GSH-Px and SOD ELISAs to determine whether antioxidant enzyme activities are altered.

5. HMVEC-L cells were treated with a phosphate buffered saline solution (PBS) negative control, 5% and 20% concentrations of ASEA and a Cachexin positive control to determine the nuclear translocation activity of the p65 subunit of NF-kB (cytokine transcription) at 30, 60, 90 and 120 min. intervals. Fluorescent microscopy techniques were employed to image cellular response.

6. Step (4) was repeated except nuclear translocation activity of P-Jun was determined as an extension/verification of step 4.

7. Two cultures of HMVEC-L cells, one with normal random cell cycles and another with serum starvation were treated with low<1% concentrations of ASEA to determine the nuclear activity of NRF2 (antioxidant transcription) at 30, 60, 90 and 120 minute intervals compared to a negative (PBS) control.

8. A Western Blot analysis was done on extra-nuclear and intra-nuclear fractions, separated by differential centrifugation, of serum starved HMVEC-L cell cultures exposed to <1% ASEA compared with a positive hydrogen peroxide control to determine phosphorylation events (oxidant action) in the extra-nuclear fraction and NRF2 (antioxidant transcription) in the intra-nuclear fraction at 0, 30, 60, 90 and 120 min. intervals.

9. Normal random cell phases of HMVEC-L cells were exposed to radiation and then treated with ASEA. Cell counts were taken to determine survival.

10. The efficacy of Cachexin reception in confluent-phase and normal-phase HMVEC-L cells was determined through changes in extracellular and intracellular LDH activity in cells exposed to various mixtures of Cachexin, PBS and ASEA solutions.

Experimental Methods used to Assess Toxic Response in Primary Human Lung Microvascular Endothelial Cells (HMVEC-L): HMVEC-L cells (catalog # CC-2527) were purchased from Lonza (Walkersville, Md.) as cryopreserved cells (Lot#7F4273). Cells were thawed and maintained according to manufacturer's directions. Cell culture medium (proprietary formulation provided by Lonza) contained epidermal growth factor, hydrocortisone, GA-1000, fetal bovine serum, vasoactive endothelial growth factor, basic fibroblast growth factor, insulin growth factor-1 and ascorbic acid.

HMVEC-L Cell cultures in normal random cell cycles were exposed to high-concentration ASEA in the serum medium, concentrations of 5% and 20%, and analyzed in conjunction with cultures exposed to phosphate buffered saline solution (PBS) as non-toxic negative control and Cachexin (5 ng/ml) as a positive control (highly toxic). At intervals of 0, 30, 60, 90, and 120 minutes, aliquots of cells from each culture were placed under a fluorescent microscope, stained by fluorescent dyes designed to tag the p65 subunit of NF-kB along with a DAPI fluorescent nuclear stain that aids the computer software to find the nuclei. Computer automated imaging techniques were used to determine the relative degree of translocation NF-kB into the nucleus via fluorescent analysis over several cells. As a reminder to the reader, P65 NF-kB translocation is the first-phase non-specific cellular response to toxicity. Thus the movement of the NF-kB into the nucleus, as seen visually in the microscope images, is a sensitive indicator of general toxic response.

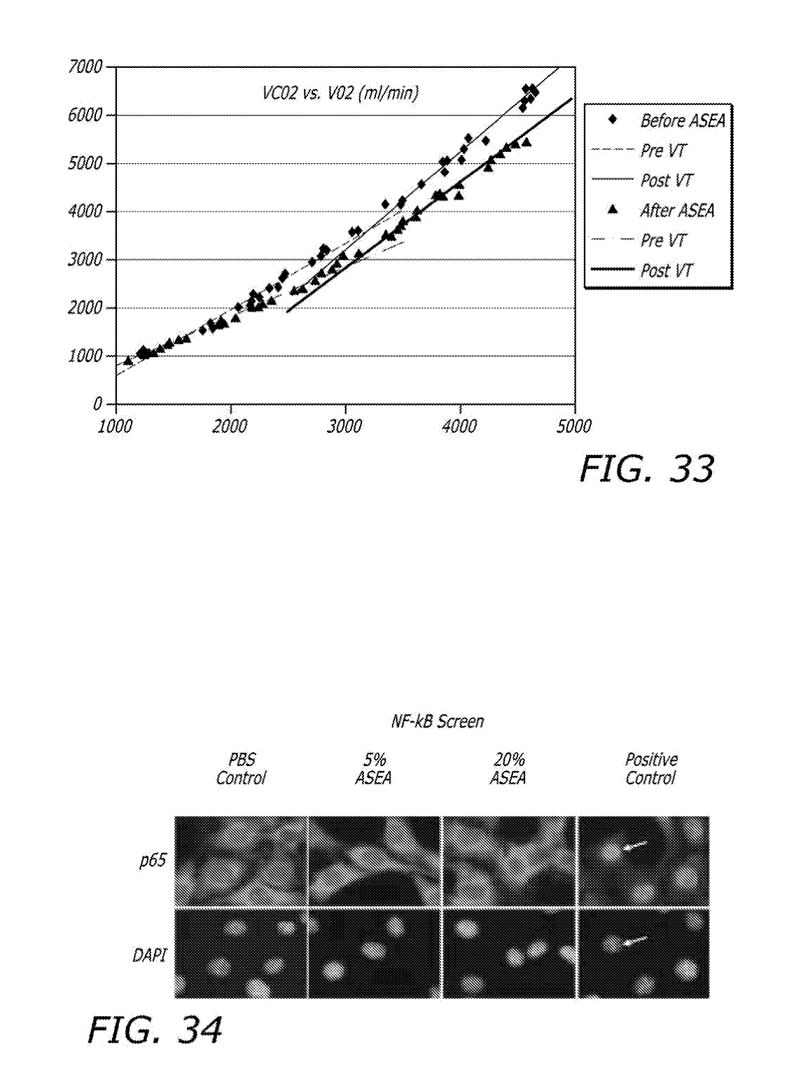

Results of HMVEC-L Cells p65 subunit NF-kB screen for toxicity: Typical cell images are shown below for each culture. Translocation of p65 subunit of NF-kB into the nucleus was not seen in any cell cultures exposed to high-concentration ASEA. Automated analysis confirmed this and indicated no toxic response at 0, 30, 90 and 120 minutes. In contrast, Cachexin exposed cells exhibited an immediate sustained toxic response (FIG. 33).

Cachexin is positive control and induces the translocation of p65 subunit of NF-kB from cytosol into nucleus. DAPI staining shows position of nuclei in these images (see white arrow). ASEA (5 and 20% final v/v) did not induce nuclear translocation of NF-kB at 30, 60 and 120 min time points.

Given this null indication of toxicity after exposure to high concentrations of ASEA, another test was performed to confirm behavior.

Additional Method to Assess Toxic Response of HMVEC-L Cells (P-Jun): A similar methodology as that employed with NF-kB was employed to determine the nuclear translocation of an anti-phospho-Jun (AP-1 P-Jun) antibody index (P-Jun is another toxicity-related redox-responsive transcription factor). HMVEC-L cells were again exposed to high-concentration ASEA. All procedures were similar to the NF-kB analysis except for the substitution of P-Jun fluorescent indicators and automated measurements taken over 100 cells in order to increase sensitivity. An additional naïve (untouched) culture was also analyzed.

Results for P-Jun screen for toxicity (FIG. 34): AP-1 index determined using anti-phospho-Jun (P-Jun) antibody. AP-1 is nuclear localized and upon activation, the phosphorylation status of P-Jun is increased. Anti-P-Jun antibody binds to the phosphorylated form reflected as an increase in fluorescence intensity (see Cachexin control). A consistent trend reflecting an increase in P-Jun levels was not observed for cells treated with 5% or 20% ASEA at 30, 60 and 120 min time points, while the Cachexin positive control significantly increased nuclear P-Jun levels at 30 min.

Again no toxic response was observed; there was no significant accumulation of P-Jun in the nuclei of cell cultures exposed to high concentrations of ASEA. Automated analysis indicated no toxic response at 0, 30, 90 and 120 minutes, with a slight but non-significant increase for 20% ASEA at the 30 minute time point; at other time points no increase was detected. In contrast, the Cachexin exposed cells (positive control), as expected exhibited an immediate sustained toxic response.

The results of the P-Jun analysis concurred with the response seen in the NF-kB analysis. For both tests, there was no significant difference between ASEA exposure and that of the negative PBS control for healthy random-phase HMVEC-L cells. This confirmed lack of toxicity was somewhat unexpected for this mixture of redox signaling molecules, considering that some of them, if isolated from the mixture, are known to elicit an immediate response.

Since nuclear translocation of NF-kB and P-Jun are typically the first responders to serum toxicity and are known to initiate the inflammatory response, especially in the ultra-sensitive human endothelial cells, healthy human cells when directly exposed to ASEA, are not expected to exhibit defensive behavior nor initiate inflammatory processes (such as the release of inflammatory cytokines). It is not certain from this data whether exposure would suppress or reverse the inflammatory process.

Blood serum levels of such redox signaling molecules, for all in vivo oral applications, would not exceed serum concentrations of 1% and typically would be less than 0.1%. Serum levels are expected to drop over time due to enzymatic breakdown of the components. Independent in vivo pharmacokinetic studies indicate that the active components in ASEA have approximately a 17 minute half-life in the blood and thus would be effectively cleared from the blood within a few hours. Thus no toxic response is expected due to exposure of healthy human cells at such levels. It has been seen in these in vitro studies that direct exposure of human cells to serum concentrations of up to 20% is still well tolerated. The complete lack of toxicity, comparable to the PBS control, is extremely rare and indicates that despite the reactivity of this mixture, it is well tolerated by human tissues and is native to or compatible with the extracellular environments.

Experimental Methods Used to Determine Antioxidant Efficacy of Glutathione Peroxidase (GPx): Cell cultures of standard murine epidermal cells (JB6), obtained locally, were exposed to various small concentrations of ASEA (less than 1%) and PBS solution for 24 hours. Cell lysates were prepared for measurements of GPx enzymatic activity using a commercially available ELISA kit (GPx activity kit, Cat #900-158) according to directions of the manufacturer (Assay Designs, Ann Arbor, Mich.). Decrease of oxidants due to GPx enzymatic activity was monitored over an 11 minute period of time after a chemical agent (cumene hydroperoxide) initiated the reaction. The decrease of oxidants is an indication of antioxidant efficacy. To determine GPx efficacy at various concentrations of PBS or ASEA, three replications of oxidant residual in the samples were read every 2 min to generate the slope, indicating the decrease in relative fluorescence units (RFU)(oxidant residual) per minute.

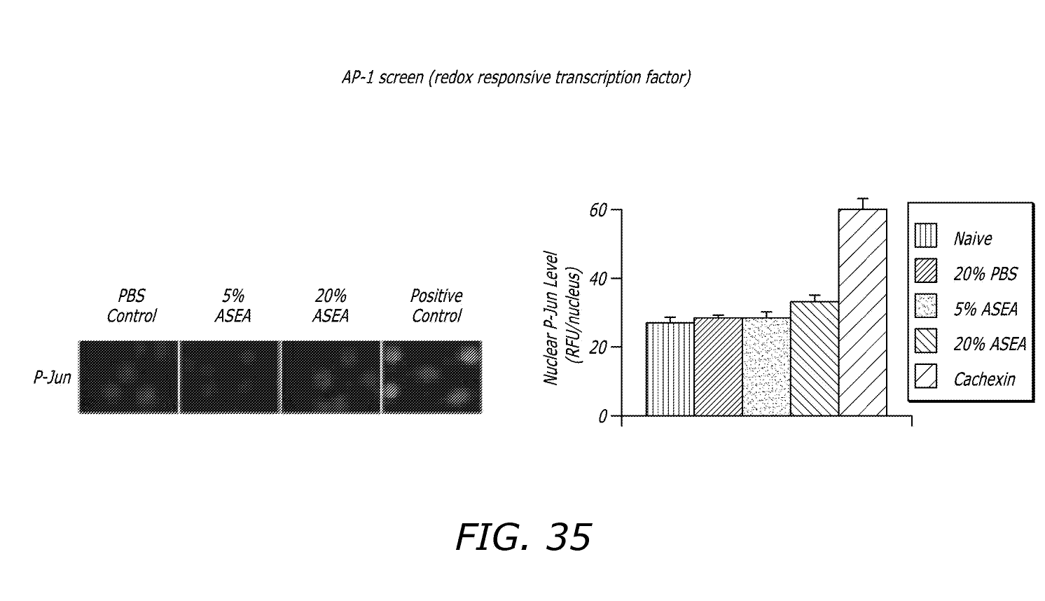

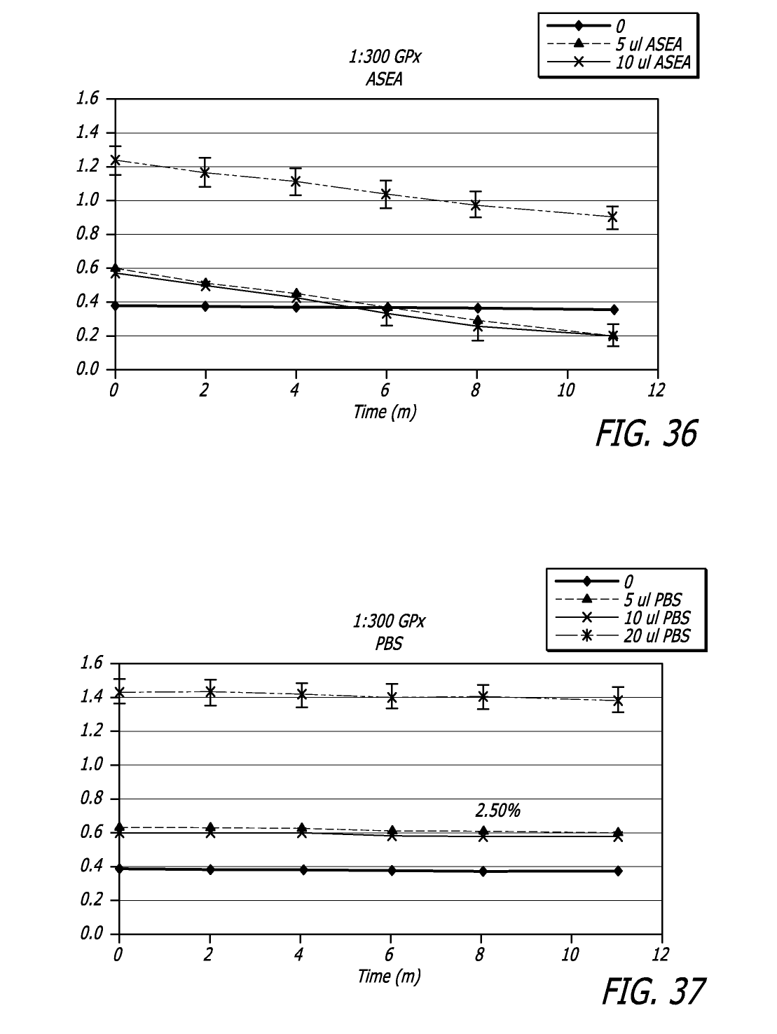

Results and Observations for GPx Antioxidant Efficacy Test: After activation, the reduction of oxidants over time was closely linear, as seen in the graphs below (RFU units on vertical scale). A well-defined slope was established over the 11 minute interval (FIG. 35). Antioxidant activity is measured by reduction of oxidants over time (FIG. 36).

A significant increase in antioxidant activity was seen in samples infused with ASEA compared to the PBS control (second graph).

Concentration dependency, however, was not seen between the 5 ul, 10 ul and 20 ul infusions. This suggests that GPx antioxidant activity might saturate at concentrations lower than that represented by the 5 ul infusion. Such considerations will be discussed later.

The table below summarizes the data shown on the preceding graphs.

| Sample Infusion Volume | Slope for PBS Control | Slope for ASEA |
|---|---|---|
| (<1% total volume) | (% reduction/minute) | (% reduction/minute) |
| 0 ul | 0.1% | 0.1% |
| 5 ul | 0.1% | 3.6% |
| 10 ul | 0.2% | 3.6% |
| 20 ul | 0.3% | 3.7% |

The raw data reflects more than a 10 fold increase in antioxidant activity related to ASEA infusion. Taking into account experimental uncertainties, it is 98% certain that the serum infusion of small concentrations (<1%) of ASEA increased antioxidant efficiencies by at least 800%. Further investigations should be done to confirm this increase and explore concentration dependence for these low-level serum concentrations.

Experimental Methods Used to Determine Antioxidant Efficacy of Superoxide Dismutase (SOD): Human HMVEC-L cells were treated with 10% phosphate buffered saline (PBS; vehicle control), 5% or 10% ASEA for 24 hr at which time cell lysates were prepared for measurements of SOD activity using a commercially available kit (SOD activity, cat#900-157) according to manufacturer's (Assay Designs, Ann Arbor, Mich.) directions. Cell culture medium was assayed for SOD activity in parallel. Limited trials with smaller concentrations of ASEA<1% and murine epidermal cells were also attempted.

Results of First-Attempt Methods to Determine SOD activity for high serum ASEA concentration: Diluted lysates showed a marginal increase in enzymatic activity associated with ASEA treatment. Changes in enzymatic activity were marginal in the initial range of 5-10% ASEA (final concentration, v/v). The data represent the first attempt to measure SOD activity using primary HMVEC-L cells treated with ASEA. It is feasible that the lack of SOD activity associated with 5-10% ASEA might be related to non-specific inhibition at high dose. The primary concern is that we have little understanding of the primary human HMVEC-L cell model and cannot determine whether these cells are optimal for investigating antioxidant defense regulation induced by ASEA. For example, ascorbic acid, known to break down certain redox signaling complexes in ASEA, is supplemented into the medium and it is feasible that some modification of the medium formula (such as omission of ascorbic acid for short periods of time defined empirically) could produce more optimal conditions for detecting antioxidant defense regulated by ASEA. Initial efforts to serum-starve these cells, as one approach to increase sensitivity and optimize the model, were unsuccessful and resulted in extensive cell death over 24 hours, indicating that the cells are dependent on the growth factors supplemented in the cell culture medium to maintain cell viability. If we interpret the initial ASEA concentrations (5-10%) to be high (based on inhibition of medium enzymatic activity and cell proliferation), then it is possible that the marginal increase in enzymatic activity associated with cell lysates observed here may not accurately reflect antioxidant defense regulation possibly occurring at lower concentrations. The use of an in vitro model system with a well defined and robust NRF2-regulated antioxidant defense response would help address some of these uncertainties. In retrospect, we have observed that a lower concentration of ASEA (1%) induces the nuclear translocation of the NRF2 transcription factor. In addition, the 24 hr time point was chosen for the initial screen as a general time point for in vitro investigations that would capture transcriptional regulation, however, this time point was not optimal.

Results of Further Investigations into SOD enzymatic activity at low ASEA concentrations (<1%): It was found in another investigation that NRF2 nuclear translocation (data and results are in the following sections), took place at low doses of ASEA (less than 1%) and elicited peak SOD antioxidant activity at about 30 to 120 minutes after exposure. Thus when SOD antioxidant activity was measured due to low-concentration ASEA exposure at a 30 to 120 minute time points, results similar to the GPx enzymatic activity were seen both with murine epidermal (JB6) cells and serum-starved HMVEC-L cells at a time point 90 to 120 minutes. Graphs were not supplied, however, a 500% increase in peak SOD enzymatic activity was estimated over a short 120 minute term, with 95% confidence.

Experimental Methods Used to Determine Nuclear Translocation of NRF2 in HMVEC-L Cells and Western Blot Verification: HMVEC-L cells were again thawed and maintained according to manufacturer's directions. The culture medium contained epidermal growth factor, hydrocortisone, GA-1000, fetal bovine serum, vasoactive endothelial growth factor, basic fibroblast growth factor, insulin growth factor-1 and ascorbic acid in randomly cycling cultures. Ascorbic acid was withheld from serum-starved cultures.

HMVEC-L Cell cultures in both normal random cell cycles and in serum starvation were exposed to high-concentration (5-20%) and low-concentration (1%) ASEA in the serum medium and analyzed in conjunction with cultures exposed only to phosphate buffered saline solution (PBS), as a negative control. At time points of 30, 60, 90, and 120 minutes, aliquots of cells from each of the cultures were placed under a fluorescent microscope, stained by a fluorescent dye designed to tag the NRF2 transcription factor along with the DAPI fluorescent nuclear stain that aids the computer software to find the nuclei. Computer automated imaging techniques were used to determine the relative degree of nuclear accumulation of NRF2 via fluorescent analysis over several cells. NRF2 regulates the transcription of a number of phase II antioxidant defense enzymes and raises the possibility that additional antioxidant defense enzymes, such as glutathione transferase, may be expressed through exposure to ASEA. Thus the accumulation of NRF2 into the nucleus, as seen visually in the microscope images, is an indicator of increased antioxidant expression in the cells.

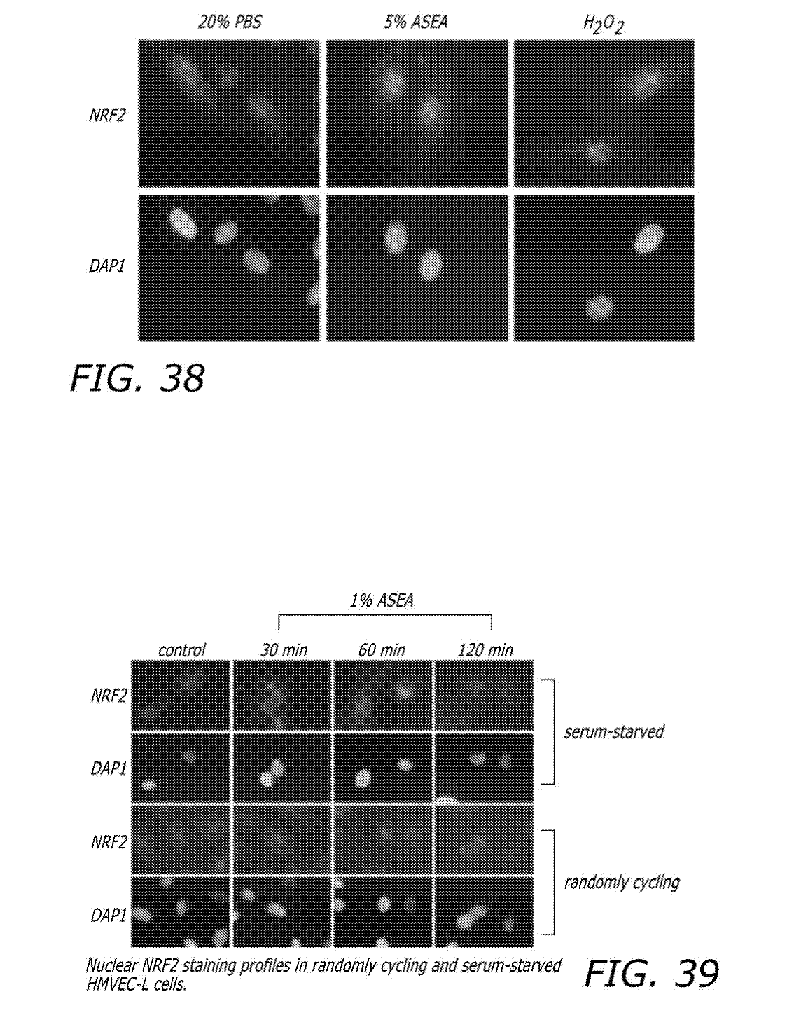

Results of HMVEC-L Nuclear Accumulation of NRF2: Initial screen of human endothelial cells suggests a subpopulation of cells showed increased nuclear staining pattern (focal) following treatment with high-concentration ASEA. The Position of nuclei are indicated by DAPI stain in lower panel. Foci appear brighter in ASEA stimulated cells which indicates higher level of NRF2 transcription factor in the nucleus. H2O2 was used as positive control. This effect was difficult to quantify based on nuclear staining pattern. Validation is required by Western blot (FIG. 38).

Typical cell images are shown below for indicated cell cultures exposed to low-concentration ASEA. Accumulation of NRF2 into the nucleus was clearly seen in serum-starved cell cultures exposed to low-concentration ASEA. Automated analysis revealed strong time-dependent nuclear accumulation of NRF2 in serum-starved cells, relative to the negative control, at the 30 and 60 minute time points (FIG. 39).

The nuclear staining profile was qualitatively different from the cells maintained in optimal growth medium (randomly cycling group). There was weak qualitative nuclear accumulation of NRF2 induced by ASEA exposure in these cells at 30, 60 and 120 minute time points, and yet the effect was not nearly as pronounced as in the serum-starved cultures. However, serum-starvation induced significant cell death complicating interpretation of the data. The trends appeared weak and require validation by Western Blot.

Experimental Methods for Western Blot Validation of NRF2 Nuclear Accumulation: HMVEC-L were treated with 1% ASEA, nuclear extracts were separated through centrifugal differentiation from the extra-nuclear cytosol at 30, 60 and 120 min and subjected to Western Blot analysis for NRF2. In the Western blot experiment the extra-nuclear fraction was probed for phosphorylated proteins using a combination of anti-phospho serine, threonine and tyrosine antibodies. Virtually all cellular processes are regulated by posttranslational modifications and protein phosphorylation is a prevalent mechanism. Observable changes in protein phosphorylation can lead to a mechanistic understanding of the cellular processes perturbed by ASEA and provide a defined endpoint to better define dose-dependent regulation of cell function by ASEA in vitro, as well as provide a potential candidate molecular marker that may be used to provide in vitro-in vivo correlates. Hydrogen peroxide (H2O2) was included as a positive control for oxidant damage.

Results for Western Blot Validation of NRF2 Nuclear Accumulation: NRF2 levels were increased in a time-dependent fashion in nuclear extracts prepared from HMVEC-L cells treated with 1% ASEA. H2O2 (30 min) did not increase nuclear NRF2 levels. In contrast, when protein phosphorylation was examined in the extra-nuclear fraction (separated from nuclei by differential centrifugation) we observed a single band by Western blot analysis and this is likely due to the dilution of the extra-nuclear fraction during the cell fractionation process (other phosphorylated proteins are obviously present but are below detection limits under these conditions) or specificity of the anti-phospho-antibodies used was insufficient to detect a broad range of phosphorylated proteins. However, we did observe a marked increase in the phosphorylation of the protein detected following H2O2 treatment, indicating that this phosphorylation event is highly sensitive to redox regulation or activation of protein kinase/deactivation of protein phosphatase activities subsequent to oxidative damage. Treatment of cells with 1% ASEA decreased phosphorylation levels associated with this protein in a time-dependent fashion (FIG. 39).

Reductions in phospho-protein regulation in extra-nuclear fractions were seen along with strong time-dependent NRF2 accumulations in the nuclear fractions, indicating clear time-dependent up-regulation of antioxidant expression.

At this point it is worth mentioning that NRF2 activity has been clearly detected in conjunction with low-concentration ASEA exposure without the normal prior NF-kB activity. This suggests that phase II antioxidant defense mechanisms have been stimulated without the normal prior phase I toxic response. This behavior has no precedent or is extremely rare. It appears from the data that ASEA is able to stimulate antioxidant expression without ever eliciting a prior low-level phase I toxic response.

Experimental Methods to Determine Proliferation of Murine (JB6) Cells and HMVEC-L Cells and LDH Activity with Exposure to ASEA: HMVEC-L cells were treated with 5-20% ASEA for 72 hr and cell number was determined using a Coulter Counter. Control (0 concentration group) was treated with 20% PBS. Serum LDH levels were also measured as an indicator of cell culture viability at 0 to 20% ASEA serum concentrations. Recall that lower serum LDH concentrations indicate less cell membrane failure. Similar experiments were performed for murine (JB6) epidermal cells.

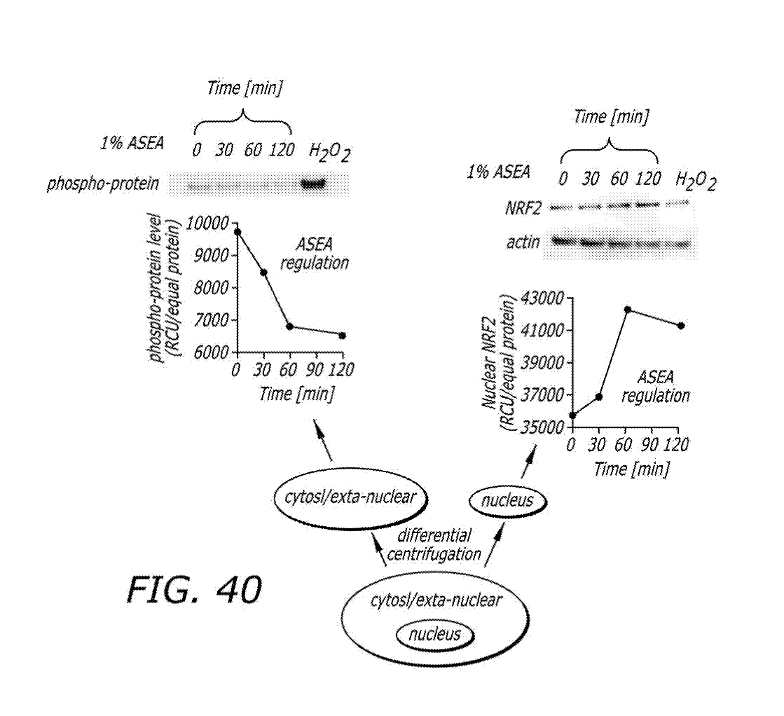

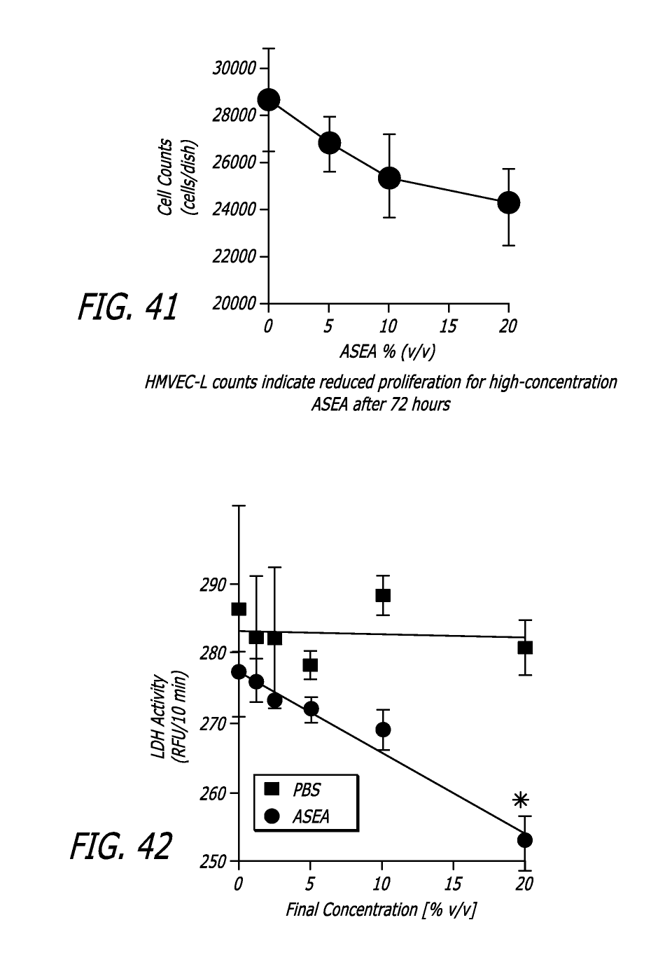

Results for Proliferation of Murine and HMVEC-L cells and LDH activity: The initial in vitro screen indicates that high-concentrations of serum ASEA may inhibit cell proliferation (for both murine epidermal cells [JB6] and primary human lung microvascular endothelial cells [HMVEC-L]) in the concentration range of 5-20%. In this concentration range we also observed direct inhibition of LDH enzymatic activity. The data are somewhat contradictory as the decreasing cell counts indicate cell death, yet lower serum LDH levels indicate higher cellular membrane integrity. At the highest concentration tested (20% v/v), cell proliferation was inhibited by approximately 20% (FIG. 40).

The mechanism behind reduced proliferation cannot be deduced and could be related to interference with growth factor responsiveness or other possible interpretations such as enhanced programmed death (apoptotic response) for damaged cells. It is noteworthy that high-concentration serum ASEA for in vitro enzymatic enhancement studies is not optimal, it is possible that the initial screens underestimated or even missed antioxidant defense (SOD) regulation by ASEA and thus indicate that low-concentration (<1%) ASEA and/or short exposure times should be employed for such purpose.

Further studies were done that investigated the action of stressed cells upon exposure to ASEA; the source of stress resulting from a variety of chemical and environmental stressors. These investigations offer clues for the possible mechanisms.

Experimental Methods to Determine cell viability of HMVEC-L exposed to various mixtures of Cachexin stressor and high-concentration ASEA: HMVEC-L cultures with normal random cell cycles (pS) and cultures approaching confluence (A2), which are generally less sensitive to Cachexin, were infused with escalating concentrations of Cachexin stressor (0-5 ng/ml). These cultures had been pretreated with either a 10% PBS control or 5-10% concentration of ASEA for 24 hours. Two indicators for cell viability were employed. Serum LDH levels were obtained as an indication of membrane integrity and Neutral Red dye was used as an indication of lysosomal integrity. Recall that as cell membranes fail, LDH is released into the serum medium. Lower quantities of LDH indicate higher cell viability. The integrity of lysosomes, necessary for viable cell function, are measured by absorption of Neutral Red dye stain. Higher quantities of Neutral Red absorbance indicate higher cell viability.

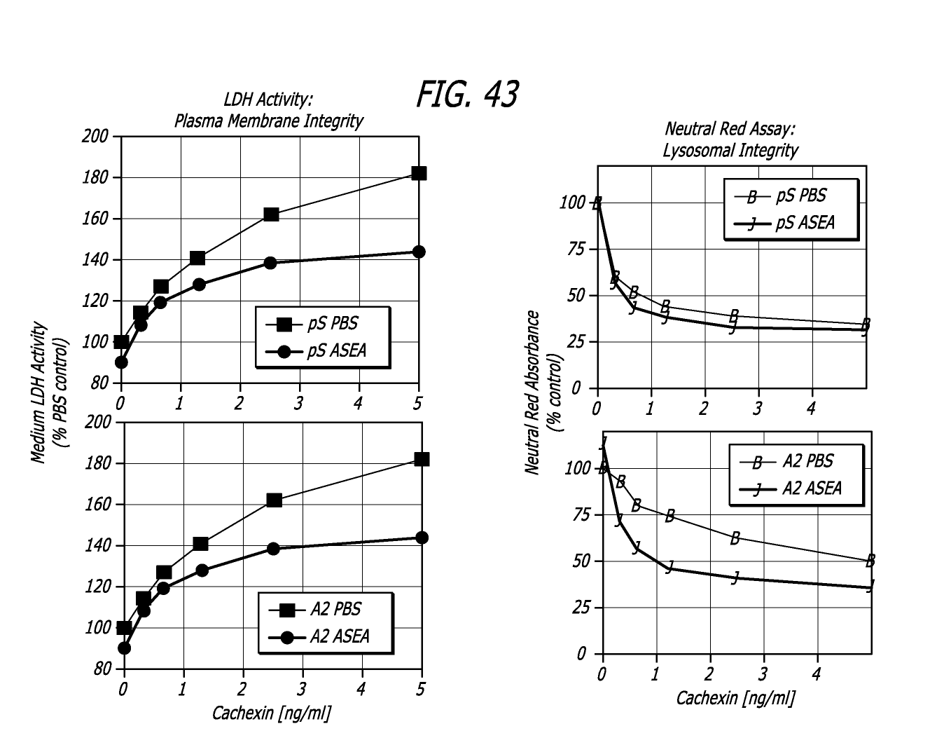

Results of HMVEC-L viability exposed high-concentration ASEA and to escalating amounts of Cachexin stressor (FIG. 43): Both confluent (A2) and normal (pS) HMVEC-L cultures exhibited up to 30% improvement (relative to PBS controls) in LDH levels related to ASEA exposure after acute (up to 5 nm/ml) Cachexin insult. The LDH data suggest that HMVEC-L cells stressed by Cachexin are less likely to die due to cell membrane failure after being exposed to ASEA.

Behavior of lysosomal integrity in HMVEC-L cells as measured by Neutral Red absorption exhibited behavior dependent on cell culture phase. As expected, the confluent (A2) cells in the PBS control were much less sensitive to Cachexin insult than cells in the PBS control normal random phase (pS) culture; this is evidenced in the 5 ng/ml Cachexin data: Lysosomal levels in A2 cells dropped only 50% compared to 70% in the pS culture. Exposure of the normal (pS) cultures to ASEA made little difference in lysosomal integrity under similar Cachexin insult, yet exposure of confluent (A2) cell cultures to ASEA made them much more sensitive to Cachexin insult, regressing to behavior similar to that exhibited by the normal more sensitive (pS) cells.

This is the first evidence presented that suggests that exposure of abnormal (Cachexin-insensitive) HMVEC-L cells to ASEA can make them more sensitive. The data suggest that confluent (A2) cells stressed by Cachexin are more likely to die when exposed to ASEA, these abnormal cells when exposed to ASEA exhibit closer to normal behavior in the presence of Cachexin. This behavior was initially unexpected as the hypothesis of the experiment was that ASEA would help cells protect themselves against toxic insult. As it turns out, it appears that ASEA exposure only helps normal healthy cells to protect themselves against oxidative insult and yet seems not to help cells protect themselves against Cachexin. ASEA exposure may even help facilitate the death of the stressed cells that are close to the end of their normal life cycle. Incidentally, the normal role of Cachexin in the tissues is to facilitate the death and replacement of damaged cells.

Experimental methods to determine the ASEA concentration-dependent response of A2 and pS phase HMVEC-L cells to Cachexin insult: HMVEC-L cell cultures, prepared in two phases, in the confluent end-of-life-cycle A2 phase (a phase typically insensitive to Cachexin insult) and in the normal random cycle pS phase were exposed for 24 hours to serum concentrations (v/v of 2.5%, 5%, 10%, 15% and 20%) of either the PBS control or ASEA. Cachexin responsiveness was then determined by monitoring LDH activity in both the intracellular cytosol and in the surrounding growth media. Recall that increased LDH activity in the growth media indicates cell membrane rupture and death (LDH release) and the decrease of intracellular LDH activity indicates loss of cellular integrity. Thus the cell cultures that are responsive to Cachexin insult would experience an increase in medium LDH activity and a decrease in intracellular LDH activity.

LDH activity in untouched cell culture controls were compared to that of cell cultures insulted with 5 ng/ml Cachexin for each ASEA concentration considered. The ASEA concentration dependence was then graphed against LDH activity for each insulted culture and control.

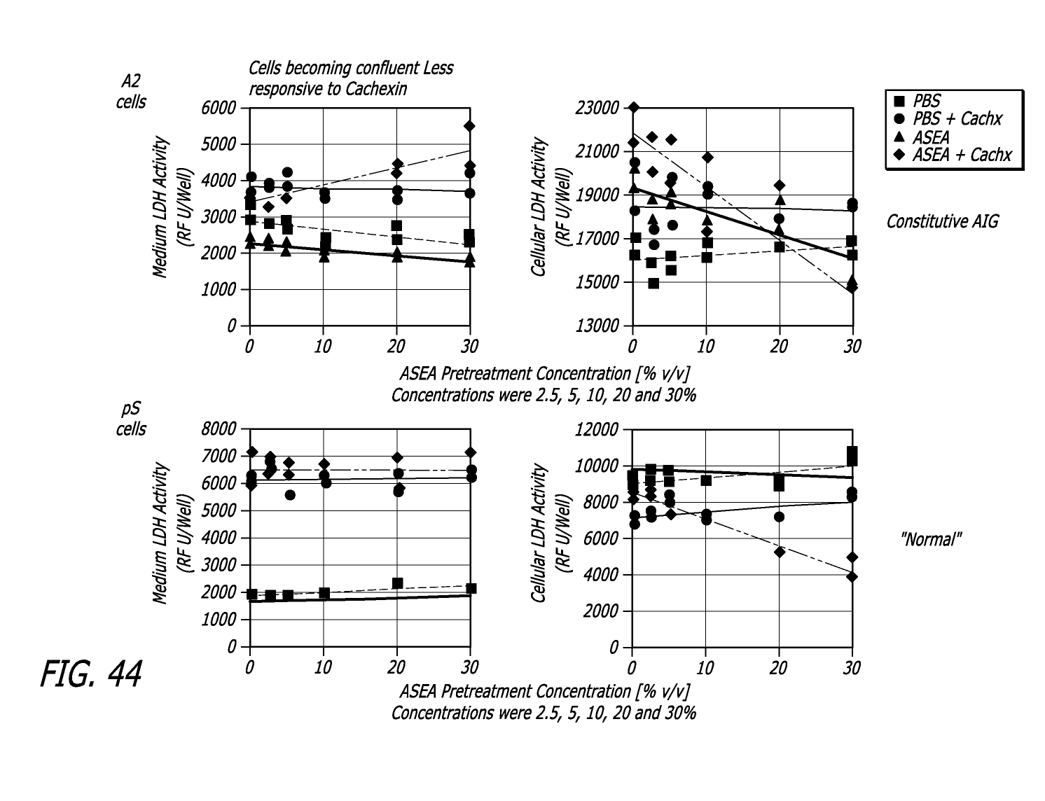

Results of concentration-dependent response of HMVEC-L cells to Cachexin insult (FIG. 44): Relative to the PBS control, the Cachexin response for the normal pS cells was much smaller than expected. Only slight decreases in cell membrane integrity were seen in the PBS control cultures and the intracellular LDH activity remained the same. With ASEA exposure, by itself, the normal pS cell cultures suffered a slight decrease in overall cellular integrity and increase in cell death. It should be noted that since the large expected response of the control pS cells to Cachexin was not manifest, it is probable that the pS cell cultures used in this investigation were nearing a confluent or non-responsive state.

There was, however, a clear response when Cachexin insult was added to the pS cell cultures exposed to various ASEA concentrations, cultures demonstrated a clear loss of intracellular LDH function and integrity. However, the accompanying indication of cell death was not seen. This seems to indicate that the “normal pS” cells were made more sensitive to Cachexin reception by ASEA exposure, yet not brought completely to the point of cell death.

The A2 cell culture response was very clear. ASEA exposure, even without Cachexin, seemed to cause loss of intracellular LDH integrity, though it did not affect cell death. However, when Cachexin insult was applied to such A2 cultures, ASEA exposure clearly amplified the Cachexin reception rapidly decreasing cellular function and there were also clear indications of concentration-dependent cell death. There is strong evidence that ASEA exposure increases Cachexin responsiveness in the A2 cell cultures.

The results imply that ASEA exposure significantly increases Cachexin responsiveness in A2 and borderline pS HMVEC-L cell cultures. Of possible interest, ASEA exposure alone might decrease integrity of cellular LDH activity in A2 type cells; recall that zero toxic response was detected in randomly cycling cells even under large concentrations, so effects due to toxicity are not expected in normal cells. It appears that ASEA exposure may tend to accelerate the removal of non-responsive confluent cells. This is evidently true when Cachexin is present. These results might also bear on the observations that ASEA exposure seemed to diminish cell proliferation in high concentrations. No such trend was tried for low-concentration exposure. Note that it is difficult to discount the possibility that high-concentration effects might simply be artifacts due to the interference of ASEA with the growth medium.

Experimental methods to determine effects of 5-10% ASEA exposure to cells stressed by radiation and serum starvation: Murine (JB6) cell cultures were subjected to high-level radiation exposure (X-rays) and, in a separate investigation, cultures were subject to serum starvation of growth factors for 24 hours. The cells were then exposed to 5-10% ASEA exposure as means to determine the effect of ASEA exposure on such stressed cells. Cell counts were taken before and after ASEA exposure.

Results of effects of 5-10% ASEA exposure on radiation and serum-starved murine cells: Quantitative analysis was not compiled for these experiments. Qualitative analysis, however, reveals results that might be of some interest. For the radiation-damaged culture, immediate cell death was observed for more than half of the culture upon exposure to ASEA. No further cell-death was seen thereafter. Upon inspection under a microscope, the remaining living cells appeared normal and healthy. It appears that ASEA exposure may have helped accelerate cell death among the more seriously damaged cells and allowed for the survival of healthy or repairable cells.

For serum-starved cell cultures similar observations were made, except the cell death was not nearly as severe, amounting to less than roughly a 20% loss. Surviving cells appeared to be very robust and viable. Similar losses, however, were also seen in serum-starved cultures that were not exposed to ASEA in later experiments.

A better understanding of the bioactivity of a certain mixture of redox signaling molecules has been determined from in vitro studies involving direct contact of ASEA with viable living HMVEC-L human cells and murine epidermal JB6 cells. Five specific objectives were pursued to determine:

1) In vitro toxicity (based on NF-kB, P-Jun translocation)

2) Effects on antioxidant efficacy (for GPx and SOD)

3) Effects on antioxidant transcriptional activity (NRF2)

4) Effects on cell proliferation and viability (cell counts)

5) Effects on stressed cells (Cachexin, radiation, starvation)

No toxic response was observed for any healthy cell culture in normal random phases (HMVEC-L or JB6) upon exposure to high concentrations (up to 20%) of serum ASEA. Two methods were used to determine toxic response, the translocation and accumulation of NF-kB and P-Jun in the nuclei. Both of these methods are known to be sensitive to low-levels of toxicity, as verified by the positive control. A complete lack of toxic indication and/or inflammatory cytokines was observed.

An 800% increase in GPx antioxidant efficacy in HMVEC-L cells was seen after 24 hours exposure from low-concentration ASEA (no concentration dependence seen). A transitory increase of up to 500% was seen in SOD antioxidant efficacy between 30 to 90 min. again after exposure to low-concentration ASEA (<1%). In both cases, the low concentrations of ASEA were comparable to blood concentrations possible from oral dosing, though data is not available to confirm this. Concentration dependence at very low concentrations might be seen if such was carefully investigated.

Exposure to high-concentration ASEA, in comparison, elicited only a small relative increase in GPx antioxidant efficacy that was not concentration dependent. An increase in SOD efficacy was not seen for either high-concentration ASEA or after long (24 hr) exposures. In subsequent investigations, this information will be used to determine optimal concentrations and time points to study concentration dependence (less than 0.1% and 0-120 minutes).

Studies examining the nuclear translocation of redox responsive transcription factors suggest that ASEA at a lower concentration (less than 1%) induces a 20-30% increase in the nuclear translocation of the NRF2 transcription factor in HMVEC-L cells that appears to be transient (30-60 min). We also observed that ASEA induced a parallel decrease in the phosphorylation of an extra-nuclear protein whose phosphorylation status is clearly increased in response to hydrogen peroxide treatment, consistent with an antioxidant mode of action.

Serum-starving HMVEC-L cells, as an approach to increase sensitivity, significantly increased the nuclear NRF2 signal induced by ASEA (1%). However, serum-starvation induced significant cell death complicating interpretation of the data.

Cellular proliferation for both HMVEC-L and JB6 cell types (determined from cell counts) was inhibited by high concentrations (5-20% v/v) of ASEA exposure. The HMVEC-L inhibition was clearly concentration dependent, with a 20% loss of cell count at 20% ASEA concentration. In contrast to decreased proliferation, serum LDH levels significantly decreased with ASEA concentration between 5-20%, indicating increased cell membrane integrity. The results seem to indicate that cellular proliferation is decreased while cell membrane viability is increased at high concentrations. The mechanism behind such behavior cannot be deduced from the data, yet further evidence will be seen in the next section.

The response of HMVEC-L cells when stressed with Cachexin depends upon cell phase. Normal randomly cycling HMVEC-L cells (pS) exhibited typical behavior when stressed with Cachexin: exhibiting decrease in cell viability accompanied by cell death. Confluent end-of-life-cycle (A2) and borderline HMVEC-L cells, as expected, were less sensitive to Cachexin insult, exhibiting less pronounced decreases in cell viability and less cell death.

Exposure to ASEA caused no significant change in the response of the normal random cycling pS cells to Cachexin (showing similar loss of cell viability and cell-death). However, A2 cell cultures exposed to ASEA exhibited increased sensitivity to Cachexin, restoring behavior similar to that of normal cells. This behavior was reinforced as ASEA concentration dependence was examined. Borderline A2 cells, exhibiting a relatively small Cachexin response, and A2 cells that are normally insensitive to Cachexin insult, exhibited a much stronger response to Cachexin when exposed to ASEA, both in decrease in viability and increased cell death.

It appears that exposure to ASEA causes increased rates of A2 cell death, enhancing the natural reception of Cachexin in such end-of-life-cycle cells. Yet exposure to ASEA is not expected to cause any change in normal cell viability.

Cachexin is normally secreted to instigate cell death in damaged or dysfunctional tissues, allowing surrounding healthy cells to divide and fill in voids. Thus, increasing the sensitivity to Cachexin in dysfunctional cells may help accelerate such a process and is not always deleterious.

Acceleration of cell death was also seen in tissues that were stressed with radiation and serum-starvation associated with exposure to ASEA.

The infusion of a certain balanced mixture of redox signaling molecules, ASEA, into viable HMVEC-L and JB6 cell cultures has been seen to elicit distinct bioactivity. No indications of toxicity or the expression of inflammatory cytokines were observed and yet there was increased antioxidant and protective enzyme expression (as evidenced by increased nuclear NRF2) and greatly increased efficacy for the two master antioxidants, GPx and SOD. This behavior suggests that ASEA infusion might tend to induce and enhance oxidative defense mechanisms without inducing toxic or inflammatory responses in such cells. Such action is unprecedented or extremely rare. Normally, low-level toxicity induces slight oxidative stress and inflammatory response which in turn induces oxidative defense and cell repair mechanisms. It would be of interest to determine concentration dependency of this effect with ultra-low-concentration ASEA infusions.

The induction of cell death in cultures of dysfunctional, stressed or damaged cells by ASEA infusion should also be explored. Natural healing processes involve a repair or replace mechanism by which marginally damaged cells are repaired, when possible, or undergo apoptosis, programmed death, if they cannot be repaired and then are replaced through mitosis of healthy neighboring cells. It is fairly evident that ASEA infusion, of itself, is not causing direct stress to exposed cells, however, it might tend to increase the efficiency of certain cytokine “death domain” messengers (Cachexin) that are designed to induce cell death in dysfunctional or damaged cells. The nuclear translocation of NRF2 can be considered part of the phase II oxidative defense response which includes expression of antioxidants, DNA repair molecules and other known repair mechanisms.

Apoptosis is part of the replace mechanism when cells have undergone unrepairable damage and must be removed and replaced. Both antioxidant defense and apoptotic mechanisms are central to normal tissue repair and regeneration. Redox signaling is involved in several of the pathways, such as p53 gene expression, that can determine whether a cell undergoes apoptosis or not. Chronic oxidative stress tends to favor cell death. Certainly the presence of Cachexin and other death domain messengers favor cell death. The observation that ASEA infusion enhances Cachexin reception might indicate that ASEA infusion also might serve to enhance reception of messengers in the signaling process that determines whether defense, repair or replace mechanisms are activated.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of forming a life enhancing beverage comprising electrolyzing salinated water having a salt concentration of about 10.75 g NaCl/gal using a set of electrodes with an amperage of about 56 amps, wherein the water is chilled below room temperature and circulated during electrolyzing.

2. The method of claim 1, wherein the set of electrodes includes 8 electrodes, and each electrode receives 7 amps of power.

3. (canceled)

4. The method of claim 1, wherein the water has less than or equal to 0.5 ppm of total dissolved solids prior to being salinated.

5. (canceled)

6. The method of claim 1, wherein the salinated water is formed using a brine solution having a NaCl concentration of about 537.5 g NaCl/gal.

7. The method of claim 1, wherein the water is chilled to a temperature of about 4.5° C. to about 5.8° C.

8. The method of claim 1, wherein the life enhancing beverage is bottled and each bottle has a saline concentration of about 0.15% w/v.

9. The method of claim 1, wherein the water is circulated at a rate of about 1.000 gal/hr.

10. The method of claim 1, wherein the life enhancing beverage comprises a reaction product consisting essentially of O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, ¹O, OH*⁻, HOCl—O₂*⁻, HOCl—O₃, O₂*⁻, HO₂*, NaCl, HCl, NaOH, and water clusters.

11. A life enhancing beverage production system comprising:
a mixing apparatus configured to mix water having less than about 0.5 ppm of total dissolved solids and a brine solution having a NaCl concentration of about 537.5 g NaCl/gal; and
at least one electrochemical tank including a 7 amp electrode, a recirculation apparatus, and a chilling apparatus configured to chill a solution being electrolyzed.

12. (canceled)

13. (canceled)

14. (canceled)

15. (canceled)

16. The life enhancing beverage production system of claim 11, further comprising a plurality of propylene glycol filled chilling tubes.

17. (canceled)

18. (canceled)

19. (canceled)

20. (canceled)

21. (canceled)

22. A method of treating a condition, the method comprising administering to a patient
a life enhancing beverage of any of claims 1-4 and 6-10.

23. The method of claim 22, wherein the condition is selected from the group consisting of decreased athletic performance, oxidative stress related disorder, reduced mitochondrial DNA, and muscle glycogen depletion.

24. The method of claim 23, wherein the administration occurs once a day.

25. The method of claim 23, wherein administration is between about 1 oz and about 16 oz per day.

26. The method of claim 23, further comprising a step for increasing time to exhaustion when exercising after administration of the life enhancing beverage.

27. (canceled)

28. The method of claim 23, wherein the administration occurs twice a day.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 1, further comprising:
forming a standard life enhancing beverage reference standard in a 1 L container using 0.9% isotonic saline solution and applying 3 amps thereto;
using the standard life enhancing beverage as a reference standard for the production of a life enhancing beverage containing at least one reaction product, wherein the reaction product includes O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, ¹O, OH*⁻, HOCl—O₂*⁻, HOCl—O₃, O₂*⁻, HO₂*, NaCl, HCl, NaOH, water clusters; and
preparing a second life enhancing beverage which has an equivalent amount the of at least one reaction product as the standard life enhancing beverage such that the standard life enhancing beverage is used as a reference standard and the amounts of the of at least one reaction product in the standard life enhancing beverage are a target amount of the of at least one reaction product for the second life enhancing beverage wherein the second life enhancing beverage is made using brine solution having a NaCl concentration of about 537.5 g NaCl/gal in tanks which hold 180 gallons.

41. The method of claim 40, wherein a pulsed current is applied when forming the life enhancing beverage and the second life enhancing beverage.

42. The method of claim 1, further comprising:
producing a life enhancing beverage standard containing at least one reaction product, wherein the reaction product includes O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, ¹O, OH*⁻, HOCl—O₂*⁻, HOCl—O₃, O₂*⁻, HO₂*, NaCl, HCl, NaOH, water clusters; and
preparing a life enhancing beverage which has an equivalent amount the of at least one reaction product as the life enhancing beverage standard such that the life enhancing beverage standard is used as a reference standard and the amounts of the of the at least one reaction product in the life enhancing beverage standard are a target amount of the of at least one reaction product for the life enhancing beverage wherein the life enhancing beverage is made using brine solution having a NaCl concentration of about 537.5 g NaCl/gal in tanks which hold 180 gallons.

43. The method of claim 42, wherein the at least one reaction product includes superoxide and chlorine.

44. (canceled)

45. (canceled)

46. (canceled)

47. (canceled)

48. (canceled)
